(19) United States
(12) Patent Application Publication
(10) Pub. No.: US 2021/0393645 A1
(43) Pub. Date: Dec. 23, 2021

(54) TREATMENT FOR PROGRESSIVE MULTIPLE SCLEROSIS

(71) Applicant: UTI LIMITED PARTNERSHIP, Calgary (CA)

(72) Inventors: Voon Wee YONG, Calgary (CA); Simon FAISSNER, Bochum (DE); Marcus KOCH, Calgary (CA); Nathan James MICHAELS, Kamloops (CA)

(21) Appl. No.: 17/358,966

(22) Filed: Jun. 25, 2021

Related U.S. Application Data
(62) Division of application No. 16/343,818, filed on Apr. 22, 2019.
(60) Provisional application No. 62/412,534, filed on Oct. 25, 2016.

(51) Int. Cl. A61K 31/55; A61P 25/28; A61K 31/404; A61P 21/00; A61K 31/5513; A61K 31/4706; A61K 31/65

(52) U.S. Cl. CPC A61K 31/55; A61P 25/28; A61K 31/404; A61K 31/65; A61K 31/5513; A61K 31/4706; A61P 21/00

(57) ABSTRACT

In one aspect, there is provided a method of treating, prophylaxis, or amelioration of a neurological disease by administering to a subject in need thereof one or more compounds described herein. In a specific example, the neurological disease is multiple sclerosis (also referred to as “MS”).

### FIELD

The present disclosure relates generally to compound(s), composition(s), and method(s) for treatment for progressive multiple sclerosis in a subject.

### BACKGROUND

Multiple sclerosis is a multifactorial inflammatory condition of the CNS leading to damage of the myelin sheath and axons/neurons followed by neurological symptoms (Ransohoff et al., 2015). Approximately 85% of multiple sclerosis patients present with a relapsing-remitting phenotype and the majority of these evolve to a secondary-progressive disease course after 15-20 years. Ten-15% of the patients experience a primary progressive disease course with slow and continuous deterioration without definable relapses.

While there have been tremendous successes in the development of medications for relapsing-remitting multiple sclerosis during the last decade, nearly all studies conducted in progressive multiple sclerosis have failed such as the recently published INFORMS study on the sphingosine-1-phosphate inhibitor fingolimod (Lublin et al., 2016). The reasons for the lack of medications in progressive multiple sclerosis are manifold.

### SUMMARY

In one aspect there is described herein a method of treating progressive multiple sclerosis comprising administering to a subject in need thereof, a therapeutically effective amount of one or more of dipyridamole, clopidogrel, cefaclor, clarithromycin, erythromycin, rifampin, loperamide, ketoconazole, labetalol, methyldopa, metoprolol, atenolol, carvedilol, indapamide, mefloquine, primaquine, mitoxanthrone, levodopa, trimeprazine, chlorpromazine, clozapine, periciazine, flunarizine, dimenhydrinate, diphenhydramine, promethazine, phenazopyridine, yohimbine, memantine, liothyronine, clomipramine, desipramine, doxepin, imipramine, trimipramine, or functional derivative thereof.

In one aspect there is described herein a method of treating progressive multiple sclerosis comprising administering to a subject in need thereof, a therapeutically effective amount of clomipramine, or a functional derivative thereof.

In one aspect there is described herein a method of treating progressive multiple sclerosis comprising administering to a subject in need thereof, a therapeutically effective amount of imipramine, or a functional derivative thereof.

In one aspect there is described herein a method of treating progressive multiple sclerosis comprising administering to a subject in need thereof, a therapeutically effective amount of trimipramine, or a functional derivative thereof.

In one aspect there is described a method of treating progressive multiple sclerosis comprising administering to a subject in need thereof, a therapeutically effective amount of clomipramine, or a functional derivative thereof, and a therapeutically effective amount of indapamide, or a functional derivative thereof.

In one aspect there is described a method of treating progressive multiple sclerosis comprising administering to a subject in need thereof, a therapeutically effective amount of indapamide, or a functional derivative thereof.

In one aspect there is described a method of treating progressive multiple sclerosis comprising administering to a subject in need thereof, a therapeutically effective amount of indapamide, or a functional derivative thereof, and one or more of hydroxychloroquine, minocycline, or clomipramine.

In one example said multiple sclerosis is primary progressive multiple sclerosis.

In one example said multiple sclerosis is secondary progressive multiple sclerosis.

In one example said multiple sclerosis is progressive relapsing multiple sclerosis.

In one example said treatment further comprises administering a therapeutically effective amount of Laquinimod, Fingolimod, Masitinib, Ocrelizumab, Ibudilast, Anti-LINGO-1, MD1003 (high concentration Biotin), Natalizumab, Siponimod, Tcelna (imilecleucel-T), Simvastatin, Dimethyl fumarate, Autologous haematopoietic stem cell transplantation, Amiloride, Riluzole, Fluoxetine, Glatiramer Acetate, Interferon Beta, or a functional derivative thereof.

In one example said subject is a human.

In one aspect there is described herein use of one or more of dipyridamole, clopidogrel, cefaclor, clarithromycin, erythromycin, rifampin, loperamide, ketoconazole, labetalol, methyldopa, metoprolol, atenolol, carvedilol, indapamide, mefloquine, primaquine, mitoxanthrone, levodopa, trimeprazine, chlorpromazine, clozapine, periciazine, flunarizine, dimenhydrinate, diphenhydramine, promethazine, phenazopyridine, yohimbine, memantine, liothyronine, clomipramine, desipramine, doxepin, imipramine, trimipramine, or functional derivative thereof, for the treatment of progressive multiple sclerosis in a subject.

In one aspect there is described herein use of one or more of dipyridamole, clopidogrel, cefaclor, clarithromycin, erythromycin, rifampin, loperamide, ketoconazole, labetalol, methyldopa, metoprolol, atenolol, carvedilol, indapamide, mefloquine, primaquine, mitoxanthrone, levodopa, trimeprazine, chlorpromazine, clozapine, periciazine, flunarizine, dimenhydrinate, diphenhydramine, promethazine, phenazopyridine, yohimbine, memantine, liothyronine, clomipramine, desipramine, doxepin, imipramine, trimipramine, or functional derivative thereof, in the manufacture of a medicament for the treatment of progressive multiple sclerosis in a subject.

In one aspect there is described herein use of clomipramine, or a functional derivative thereof, for treating progressive multiple sclerosis in a subject in need thereof.

In one aspect there is described herein use of clomipramine, or a functional derivative thereof, in the manufacture of a medicament for treating progressive multiple sclerosis in a subject in need thereof.

In one aspect there is described herein use of imipramine, or a functional derivative thereof, for treating progressive multiple sclerosis in a subject in need thereof.

In one aspect there is described herein use of imipramine, or a functional derivative thereof, in the manufacture of a medicament for treating progressive multiple sclerosis in a subject in need thereof.

In one aspect there is described herein use of trimipramine, or a functional derivative thereof, for treating progressive multiple sclerosis in a subject in need thereof.

In one aspect there is described herein use of a therapeutically effective amount of trimipramine, or a functional derivative thereof, in the manufacture of a medicament for treating progressive multiple sclerosis in a subject in need thereof.

In one aspect, there is described a use of clomipramine, or a functional derivative thereof, and a use of indapamide, or a functional derivative thereof, for treating progressive multiple sclerosis in subject in need thereof.

In one aspect there is described a use of clomipramine, or a functional derivative thereof, and a use of indapamide, or a functional derivative thereof, in the manufacture of a medicament for treating progressive multiple sclerosis in subject in need thereof.

In one aspect, there is described a use of indapamide, or a functional derivative thereof, for treating progressive multiple sclerosis in subject in need thereof.

In one aspect, there is described a use of indapamide, or a functional derivative thereof, in the manufacture of a medicament for treating progressive multiple sclerosis in subject in need thereof.

In one aspect, there is described a use of indapamide, or a functional derivative thereof, and one or more of hydroxychloroquine, minocycline, or clomipramine, or a functional derivative thereof, for treating progressive multiple sclerosis in subject in need thereof.

In one aspect, there is described a use of indapamide, or a functional derivative thereof, and one or more of hydroxychloroquine, minocycline, or clomipramine, or a functional derivative thereof, in the manufacture of a medicament for treating progressive multiple sclerosis in subject in need thereof.

In one example said multiple sclerosis is primary progressive multiple sclerosis.

In one example said multiple sclerosis is secondary progressive multiple sclerosis.

In one example said multiple sclerosis is progressive relapsing multiple sclerosis.

In one example further comprising a use of a therapeutically effective amount of Laquinimod, Fingolimod, Masitinib, Ocrelizumab, Ibudilast, Anti-LINGO-1, MD1003 (high concentration Biotin), Natalizumab, Siponimod, Tcelna (imilecleucel-T), Simvastatin, Dimethyl fumarate, Autologous haematopoietic stem cell transplantation, Amiloride, Riluzole, Fluoxetine, Glatiramer Acetate, Interferon Beta, or a functional derivative thereof, for the treatment of progressive multiple sclerosis, primary progressive multiple sclerosis, or secondary multiple sclerosis.

In one example further comprising a use of a therapeutically effective amount of Laquinimod, Fingolimod, Masitinib, Ocrelizumab, Ibudilast, Anti-LINGO-1, MD1003 (high concentration Biotin), Natalizumab, Siponimod, Tcelna (imilecleucel-T), Simvastatin, Dimethyl fumarate, Autologous haematopoietic stem cell transplantation, Amiloride, Riluzole, Fluoxetine, Glatiramer Acetate, Interferon Beta, or a functional derivative thereof, in the manufacture of a medicament for the treatment of progressive multiple sclerosis, primary progressive multiple sclerosis, or secondary multiple sclerosis.

In one example the subject is a human.

In one aspect there is described herein a method of identifying a compound for the treatment of progressive multiple sclerosis, comprising: selecting one or more compounds from a library of compounds that prevent or reduce iron-mediated neurotoxicity in vitro,

selecting one or more compounds from step (a) that prevent or reduce mitochondrial damage in vitro; selecting one or more compounds from step (a) for anti-oxidative properties,

selecting one or more compound from step (a) for ability to reduce T-cell proliferation in vitro, optionally, after step (a), selecting a compound from step (a) which is predicted or known to be able to cross the blood brain barrier, or having a suitable side effect profile, or having a suitable tolerability.

In one aspect there is described herein a kit for the treatment of progressive multiple sclerosis, comprising: one or more of dipyridamole, clopidogrel, cefaclor, clarithromycin, erythromycin, rifampin, loperamide, ketoconazole, labetalol, methyldopa, metoprolol, atenolol, carvedilol, indapamide, mefloquine, primaquine, mitoxanthrone, levodopa, trimeprazine, chlorpromazine, clozapine, periciazine, flunarizine, dimenhydrinate, diphenhydramine, promethazine, phenazopyridine, yohimbine, memantine, liothyronine, clomipramine, desipramine, doxepin, imipramine, trimipramine, or functional derivative thereof and Instructions for the use thereof.

In one aspect there is described herein a kit for the treatment of progressive multiple sclerosis comprising: a therapeutically effective amount of clomipramine, or a functional derivative thereof, and instructions for use.

In one aspect there is described herein a kit for the treatment of progressive multiple sclerosis comprising: a therapeutically effective amount of imipramine, or a functional derivative thereof, and instructions for use.

In one aspect there is described herein a kit for the treatment of progressive multiple sclerosis comprising: a therapeutically effective amount of trimipramine, or a functional derivative thereof, and instructions for use.

In one aspect there is described a kit for the treatment of progressive multiple sclerosis comprising: a therapeutically effective amount of clomipramine, or a functional derivative thereof, a therapeutically effective amount indapamide, or a functional derivative thereof, and instructions for use.

In one aspect there is described a kit for the treatment of progressive multiple sclerosis comprising: a therapeutically effective amount of indapamide, or a functional derivative thereof, or a functional derivative thereof, and instructions for use.

In one aspect there is described a kit for the treatment of progressive multiple sclerosis comprising: a therapeutically effective amount of indapamide, or a functional derivative thereof, and one or more of hydroxychloroquine, minocycline, or clomipramine, or a functional derivative thereof; and instructions for use.

In one example said multiple sclerosis is primary progressive multiple sclerosis.

In one example said multiple sclerosis is secondary progressive multiple sclerosis.

In one example said multiple sclerosis is progressive relapsing multiple sclerosis.

In one example further comprising one or more of Laquinimod, Fingolimod, Masitinib, Ocrelizumab, Ibudilast, Anti-LINGO-1, MD1003 (high concentration Biotin), Natalizumab, Siponimod, Tcelna (imilecleucel-T), Simvastatin, Dimethyl fumarate, Autologous haematopoietic stem cell transplantation, Amiloride, Riluzole, Fluoxetine, Glatiramer Acetate, Interferon Beta, or a functional derivative thereof.

Other aspects and features of the present disclosure will become apparent to those ordinarily skilled in the art upon review of the following description of specific embodiments in conjunction with the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present disclosure will now be described, by way of example only, with reference to the attached Figures.

FIGS. 1A-1C: Screening of generic compounds to prevent iron mediated neurotoxicity. Shown is an example of a screening of drugs to identify those that prevent iron mediated neurotoxicity to human neurons. Neurons were pretreated with drugs at a concentration of 10 μM, followed by a challenge with 25 or 50 μM FeSO₄ after 1 h. In this experiment, several compounds (yellow bars) prevented against iron mediated neurotoxicity (FIG. 1A). Values in A are mean±SEM of n=4 wells per condition. One-way analysis of variance (ANOVA) with Bonferroni post-hoc analysis vs. iron: *p<0.05; **p<0.01; ***p<0.001; ****p<0.0001. Representative images show the control and iron treated neurons, as well as the prevention of neurotoxicity by treatment with indapamide (FIG. 1B bright field, FIG. 1C fluorescence microscopy). Neurons were detected by anti-microtubule-associated protein-2 (MAP-2) antibody. The scale bars depict 100 μm.

FIGS. 2A-2B: Summary of compounds that attenuate iron mediated neurotoxicity. Shown are all 35 generic drugs that prevent iron mediated neurotoxicity (FIG. 2A). The number of neurons in each well of a given experiment was normalized to the number of neurons of the respective untreated control condition (100%). The corresponding FeSO₄ treated condition (red) was also normalized to the respective control. Some of the major drug classes are depicted in the figure. Shown are the mean±SEM of 2-4 independent experiments, performed in quadruplicates (thus, 8-16 wells per treatment across experiments are depicted in the figure). FIG. 2B shows the results from live cell imaging of neurons challenged with FeSO4 in a concentration of 50 μM. Upon pre-treatment with indapamide or desipramine 1 h before the addition of iron, the number of propidium-iodide positive cells was significantly reduced after 7.5 h and even below the level of the control condition after 12 h, suggesting a strong neuroprotective effect. Live cell imaging was performed over 12 h, where images were taken every 30 min. The time-point from which significant changes were observed is marked with a symbol (# control; +DMSO; * indapamide; desipramine). Shown are means±SEM of n=3 wells per condition. Results were analyzed with a two-way ANOVA with Dunnett's multiple comparison as post-hoc analysis.

FIGS. 3A-3B: Prevention of mitochondrial damage induced by rotenone. Some of the generic drugs that prevented against iron mediated neurotoxicity were tested against mitochondrial damage to neurons. Some compounds, such as indapamide, prevented mitochondrial damage as shown after normalization to the control neurons (FIG. 3A). The rescue effect was however small. Treatment with rotenone induced marked morphological changes with retraction of cell processes (FIG. 3B). The scale bar shows 100 μM. Shown are normalized data of mean±SEM of 1-3 experiments each performed in quadruplicates. Two-way analysis of variance (ANOVA) with Bonferroni multiple comparisons test as post-hoc analysis vs. rotenone: *p<0.05; **p<0.01; ***p<0.001; ****p<0.0001.

FIGS. 4A-4C: Scavenging of hydroxyl radicals in a biochemical assay. The anti-oxidative capacities of selected compounds that reduced iron mediated neurotoxicity were analyzed using the hydroxyl radical antioxidant capacity (HORAC) assay. FIG. 4A shows a representative experiment depicting the decay of relative fluorescence units (RFU) over 60 min for indapamide, gallic acid (GA) and the control (blank). (FIG. 4B) The upward shift of the curve for clomipramine in the HORAC assay indicates an anti-oxidative effect that is even stronger than gallic acid. HORAC gallic acid equivalents (GAEs) were calculated by the integration of the area under the curve of the decay of fluorescence of the test compound over 60 min in comparison to 12.5 μM gallic acid and blank. Shown are data of n=3-4 independent experiments±SEM, with each experiment performed in triplicates (FIG. 4C). The antipsychotics showed strong anti-oxidative effects, as demonstrated with HORAC GAEs of >3. Data points >1 represent anti-oxidative capacity (the gallic acid effect is 1), 0 represents no anti-oxidative properties, and data <0 show pro-oxidative effect. RFU: Relative fluorescence units. Two-way analysis of variance (ANOVA) with Dunnett's multiple comparisons test as posthoc-analysis (a, b); the first significant time point vs. gallic acid is depicted as *. One-way analysis of variance (ANOVA) with Dunnett's multi comparisons test as post-hoc analysis vs. gallic acid. *p<0.05; **p<0.01; ***p<0.001; ****p<0.0001.

FIG. 5: Effects on proliferation of T-lymphocytes. The tricyclic antidepressants (clomipramine, desipramine, imipramine, trimipramine and doxepin) reduced proliferation of T-cells markedly (p<0.0001). Data were normalized to counts per minute (cpm) of activated control T-cells. Shown are data pooled from 2 independent experiments each performed in quadruplicates. Data are depicted as mean±SEM. One-way analysis of variance (ANOVA) with Dunnett's multiple comparisons test as post-hoc analysis compared to activated splenocytes. *p<0.05; **p<0.01; ***p<0.001; ****p<0.0001.

FIGS. 6A-6H: Clomipramine reduces iron neurotoxicity and proliferation of T- and B-lymphocytes. Clomipramine attenuated iron mediated neurotoxicity in a concentration-dependent manner from 100 nM (p<0.005) (FIG. 6A). Washing away clomipramine led to cell death by iron, but this effect could be prevented after pre-incubation of clomipramine with iron, suggesting a physical reaction between clomipramine and iron (FIG. 6B). Live cell imaging studies show that the increasing accumulation of PI-positive neurons exposed to iron over time was prevented by clomipramine (FIG. 6C). Clomipramine furthermore reduced the proliferation of T-lymphocytes (FIG. 6D), reflected by a reduction of cells in S-phase and an increase in the G1-phase of the cell cycle (FIG. 6E, FIG. 6F). Proliferation of activated B-Cells was reduced by clomipramine from 2 μM (FIG. 6G), correspondent with reduced TNF-α release (FIG. 6H). Data are shown as quadruplicate replicate wells of an individual experiment that was conducted twice (FIG. 6A, FIG. 6D, FIG. 6E, FIG. 6F), once (FIG. 6B) of three times (FIG. 6G, FIG. 6H); FIG. 6C represent triplicate wells of one experiment. Results are mean±SEM. One-way analysis of variance (ANOVA) with Dunnett's multiple comparisons test as post-hoc analysis compared to the FeSO4 or activated condition (FIG. 6A, FIG. 6B, FIGS. 6D-6H) and two-way analysis of variance (ANOVA) with Dunnett's multiple comparisons test (c): *p<0.05; **p<0.01; ***p<0.001; ****p<0.0001.

FIGS. 7A-7B: Clomipramine initiated from day 5 delays the onset of EAE clinical disease. Female C57BL/6 mice (age 8-10 weeks) were treated with clomipramine IP (25 mg/kg) or PBS (vehicle) from day 5 after induction of MOG-EAE (FIG. 7A). The disease onset was delayed and from day 11 the clinical course differed significantly (p<0.001). Eventually, clomipramine treated mice also developed the same disease burden as vehicle-treated mice. The overall disease burden is shown in FIG. 7B. N=8 vehicle and n=8 clomipramine EAE mice. Data are depicted as mean±SEM. Two-way ANOVA with Sidak's multiple-comparisons test as post-hoc analysis (FIG. 7A) and two-tailed unpaired non-parametric Mann-Whitney test (FIG. 7B). Significance is shown as *p<0.05; **p<0.01; ***p<0.001; ****p<0.0001.

FIGS. 8A-8F: Early clomipramine treatment suppressed EAE disease activity. Female C57BL/6 mice (age 8-10 weeks) were treated with clomipramine IP (25 mg/kg) or PBS (vehicle) from the day of induction of MOG-EAE (day 0). From day 11 the clinical course differed significantly (p<0.05); while vehicle-treated mice accumulated progressive disability, clomipramine treated mice remained unaffected even up to the termination of experiment when vehicle-treated mice were at peak clinical severity (paralysis or paresis of tail and hind limb functions, and paresis of forelimbs) (FIG. 8A). The overall burden of disease per mouse was plotted in FIG. 8B, while the relative weight of mice, reflecting general health, is shown in FIG. 8C. In the lumbar cord, at animal sacrifice (day 15), there was a significant upregulation in vehicle-EAE mice of transcripts encoding lfng, Tnfa, 11-17 and Ccl2 compared to naïve mice, whereas clomipramine treated mice did not show these elevations (FIG. 8D). Levels of clomipramine and the active metabolite desmethylclomipramine in serum and spinal cord at sacrifice (FIG. 8E) are consistent to concentrations reached in humans. There was a strong correlation of serum levels of clomipramine and desmethylclomipramine with spinal cord levels (FIG. 8F). Data in FIG. 8D are RT-PCR results, with values normalized to Gapdh as housekeeping gene and expressed in relation to levels in naïve mice. N=8 (vehicle) and n=7 (clomipramine) EAE mice. Data are depicted as mean±SEM. Two-way ANOVA with Sidak's multiple-comparisons test as post-hoc analysis (FIG. 8A), two-tailed unpaired non-parametric Mann-Whitney test (FIG. 8B), two-tailed unpaired t-test (FIG. 8C, FIG. 8E, FIG. 8F) and one-way ANOVA with Tukey's multiple comparisons test as post-hoc analysis (FIG. 8D). Correlations were calculated using a linear regression model, dotted lines show the 95%-confidence interval (FIG. 8F). Significance is shown as *p<0.05; **p<0.01; ***p<0.001; ****p<0.0001.

FIGS. 9A-9K: Reduced inflammation and axonal damage upon clomipramine treatment. Vehicle-treated animals had marked parenchymal inflammation, indicated by an arrow (FIG. 9A), whereas clomipramine-treated animals only had low meningeal inflammation (FIG. 9B). This was reflected in better histological scores (FIG. 9G) evaluated by a previously described method (Goncalves DaSilva and Yong, Am J Pathol 174:898-909, 2009) (a, b: Hematoxylin/eosin and luxol fast blue, HE & LFB). Vehicle-treated animals had pronounced microglial activation (lba1 stain, FIG. 9C), which was accompanied by axonal damage with formation of axonal bulbs (indicated by an arrow, Bielschowsky stain, FIG. 9E Clomipramine treatment reduced microglial activation concomitant with preserved axonal integrity (FIG. 9D, FIG. 9F). This was reflected in a blinded rank order analysis (FIG. 9H, FIG. 9I). Infiltration and microglial activation positively correlated with axonal damage (FIG. 9J, FIG. 9K). FIGS. 9C and 9E and FIGS. 9D and 9F are adjacent sections. Images are shown in 20- and 40-times original magnification. The scale bars show 100 μm. Non-parametric two-tailed Mann-Whitney test (FIGS. 9G-9I) and non-parametric two-tailed Spearman correlation with 95% confidence interval (FIG. 9J, FIG. 9K). Significance is shown as **p<0.01; ***p<0.001.

FIGS. 10A-10D: Clomipramine improves the chronic phase of EAE. FIG. 10A) Female C57BL/6 (age 8-10 weeks) MOG-immunized mice were treated with clomipramine IP (25 mg/kg) or PBS (vehicle) from remission after the first relapse, and this did not affect disease score between the groups (n=10 vehicle, n=10 clomipramine). FIG. 10B) In a second experiment, MOG-immunized C57BL/6 mice were treated from onset of clinical signs. Here, clomipramine reduced the clinical severity of the first relapse (day 14-20, p=0.0175, two-tailed Mann-Whitney t-test) and of the second relapse at the late chronic phase (day 42-50, p=0.0007, two-tailed Mann-Whitney t-test) (n=5 vehicle, n=6 clomipramine). Note that an initial two-way ANOVA with Sidak's multiple-comparisons test of the experiment from day 13 to 50 was not statistically significant, since vehicle-treated mice spontaneously remitted to a very low disease score between days 25 and 42, so that differences with the treatment group could not be detected. Hence, we analyzed differences of the acute and chronic relapse phases outside of the period of remission, using Mann-Whitney t-test. FIG. 10C) Using Biozzi ABH mice, treatment from onset of clinical disability showed a positive effect on the chronic phase (p =0.0062, two-tailed Mann-Whitney test) (n=5 vehicle, n=5 clomipramine). When a two-way ANOVA with Sidak's multiple-comparisons test was used, the results were not significant since the individual variability of mice in either group in any given day was very high for this model in our hands. FIG. 10D) A summary of the effect of clomipramine when treatment is initiated at the onset of clinical signs.

FIGS. 11A-11M: Shown are all 249 generic compounds of the iron mediated neurotoxicity screening (FIG. 11A-FIG. 11M). The number of neurons left following exposure to each compound was normalized to the number of neurons of the respective control condition. The corresponding iron situation was also normalized to the respective control (red). Compounds which exhibit significant protection are highlighted in yellow and marked (X). Shown are the means±SEM of 1-4 experiments, performed in quadruplicates each.

FIGS. 12A-12C show Lysolecithin deposited in the ventrolateral white matter of the mouse spinal cord produces a larger volume of demyelination in aging 8-10 month versus 6 weeks old young mice. FIG. 12A shows the greater spread of demyelination (loss of blue in the ventrolateral white matter) across multiple sections rostral (R, numbers are um distance) from the lesion epicenter (which is the bottom-most section here of a representative young and aging mouse), which manifests as a larger volume of myelin loss in aging mice (FIG. 12B). *p<0.01; **p<0.001. FIG. 12C represents the average myelin loss rostral and caudal to the epicenter in both age groups.

FIGS. 13A-13B show Greater axonal loss following lysolecithin demyelination in aging mice. FIG. 13A) Axons are visualized by an antibody to neurofilaments (SMI312) in normal appearing white matter (NAWM) and in the lesion, with fewer axons spared in lesions of aging samples at 72 h (FIG. 13B). Note that the data in FIG. 13B represent remaining axonal number in the injured ventral column expressed as a % to the counts in the uninjured ventral column. Two-tailed t-test.

FIGS. 14A-14D show RNAseq data of 3day laser-microdissected lesions that homed onto NADPH oxidase. FIG. 14A) Heat map (3 samples/group, where each sample is a pool of 5 mice) after lysolecithin (LPC) lesion in young and aging mice. FIG. 14B) Upregulation of canonical immune-associated pathways in aging vs young mice that converge, through Ingenuity Pathway Analysis (FIG. 14C), into NADPH oxidase 2 subunits. FIG. 14D) The RNAseq levels of the catalytic subunit of NADPH oxidase 2, gp91phox (also called CYBB) are selected for display. *p<0.05.

FIGS. 15A-15C show higher expression of gp91^{phix} and malondialdehyde in aging lesions. FIGS. 15A-15B) The catalytic subunit of NOX2, gp91phox, is readily found within CD45+ cells in aging but not young demyelinated lesions (d3). (FIGS. 15C-15D). Similarly, malondialdehyde as a marker of oxidative damage is in aging lesion associated with MBP+ myelin breakdown.

FIGS. 16A-16E show indapamide treatment of aging mice after lysolecithin injury results at 72 h in a smaller demyelinated volume, less axonal loss, and lower lipid peroxidation. Indapamide (20 mg/kg) was given ip immediately after demyelination, and once/day 24 h apart for the next 2 days, and mice were then killed on day 3. Impressively, indapamide reduced the volume of demyelination (FIGS. 16A-16B) and preserved axons (FIGS. 16C-16D), likely through the reduction of free radical toxicity as manifested by the lower accumulation of malondialdehyde in demyelinated mice (FIG. 16E).

### DETAILED DESCRIPTION

In one aspect, there is provided a method of treating, prophylaxis, or amelioration of a neurological disease by administering to a subject in need thereof one or more compounds described herein. In a specific example, the neurological disease is multiple sclerosis (also referred to as “MS”).

The term “multiple sclerosis” refers to an inflammatory disease of the central nervous system (CNS) in which the insulating covers of nerve cells in the brain and spinal cord are damaged. This damage disrupts the ability of parts of the nervous system to communicate, resulting in a wide range of signs and symptoms, including physical, mental, and psychiatric.

In one example, as described herein there is provided a treatment for multiple sclerosis in a subject.

As used herein, “multiple sclerosis” includes multiple sclerosis or a related disease, and optionally refers to all types and stages of multiple sclerosis, including, but not limited to: benign multiple sclerosis, relapsing remitting multiple sclerosis, secondary progressive multiple sclerosis, primary progressive multiple sclerosis, progressive relapsing multiple sclerosis, chronic progressive multiple sclerosis, transitional/progressive multiple sclerosis, rapidly worsening multiple sclerosis, clinically-definite multiple sclerosis, malignant multiple sclerosis, also known as Marburg's Variant, and acute multiple sclerosis. Optionally, “conditions relating to multiple sclerosis” include, e.g., Devic's disease, also known as Neuromyelitis Optica; acute disseminated encephalomyelitis, acute demyelinating optic neuritis, demyelinative transverse myelitis, Miller-Fisher syndrome, encephalomyelradiculoneuropathy, acute demyelinative polyneuropathy, tumefactive multiple sclerosis and Balo's concentric sclerosis.

In a specific example, the neurological disease is progressive multiple sclerosis.

In a specific example, as described herein there is provided a treatment for progressive multiple sclerosis in a subject.

As used herein, “progressive” multiple sclerosis refers to forms of the disease which progress towards an ever-worsening disease state over a period of time. Progressive multiple sclerosis includes, but is not limited to, for example, primary progressive multiple sclerosis, secondary progressive multiple sclerosis, and progressive relapsing multiple sclerosis.

These subtypes may or may not feature episodic flare-ups of the disease, but are each associated with increased symptoms, such as increased demyelination or pain and reduced capacity for movement, over time.

The term “subject”, as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. In a specific example, the subject is a human.

The term “treatment” or “treat” as used herein, refers to obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject in the early stage of disease can be treated to prevent progression or alternatively a subject in remission can be treated with a compound or composition described herein to prevent progression.

In some examples, treatment results in prevention or delay of onset or amelioration of symptoms of a disease in a subject or an attainment of a desired biological outcome, such as reduced neurodegeneration (e.g., demyelination, axonal loss, and neuronal death), reduced inflammation of the cells of the CNS, or reduced tissue injury caused by oxidative stress and/or inflammation in a variety of cells.

In some examples, treatment methods comprise administering to a subject a therapeutically effective amount of a compound or composition described herein and optionally consists of a single administration or application, or alternatively comprises a series of administrations or applications.

The term “pharmaceutically effective amount” as used herein refers to the amount of a compound, composition, drug or pharmaceutical agent that will elicit the biological or medical response of a tissue, system, animal or human that is being sought by a researcher or clinician, for example, the treatment of progressive multiple sclerosis. This amount can be a therapeutically effective amount.

The compounds and compositions may be provided in a pharmaceutically acceptable form.

The term “pharmaceutically acceptable” as used herein includes compounds, materials, compositions, and/or dosage forms (such as unit dosages) which are suitable for use in contact with the tissues of a subject without excessive toxicity, irritation, allergic response, or other problem or complication, commensurate with a reasonable benefit/risk ratio. Each carrier, excipient, etc. is also “acceptable” in the sense of being compatible with the other ingredients of the formulation.

In one example, there is provided a method of treating progressive multiple sclerosis comprising administering to a subject in need thereof, a therapeutically effective amount of one or more of dipyridamole, clopidogrel, cefaclor, clarithromycin, erythromycin, rifampin, loperamide, ketoconazole, labetalol, methyldopa, metoprolol, atenolol, carvedilol, indapamide, mefloquine, primaquine, mitoxanthrone, levodopa, trimeprazine, chlorpromazine, clozapine, periciazine, flunarizine, dimenhydrinate, diphenhydramine, promethazine, phenazopyridine, yohimbine, memantine, liothyronine, clomipramine, desipramine, doxepin, imipramine, trimipramine, or functional derivative thereof.

In a specific example, there is provided a method of treating progressive multiple sclerosis comprising administering to a subject in need thereof, a therapeutically effective amount of clomipramine, or a functional derivative thereof.

In a specific example, there is provided a method of treating multiple sclerosis comprising administering to a subject in need thereof, a therapeutically effective amount of imipramine, or a functional derivative thereof.

In a specific example, there is provided a method of treating multiple sclerosis comprising administering to a subject in need thereof, a therapeutically effective amount of trimipramine, or a functional derivative thereof.

In a specific example, there is provided a method of treating multiple sclerosis comprising administering to a subject in need thereof, a therapeutically effective amount of indapamine, or a functional derivative thereof.

In a specific example, there is provided a method of treating multiple sclerosis comprising administering to a subject in need thereof, a therapeutically effective amount of indapamine, or a functional derivative thereof, and one or more of hydroxychloroquine, minocycline, or clomipramine, or a functional derivative thereof.

The term “functional derivative” and “physiologically functional derivative” as used herein means an active compound with equivalent or near equivalent physiological functionality to the named active compound when used and/or administered as described herein. As used herein, the term “physiologically functional derivative” includes any pharmaceutically acceptable salts, solvates, esters, prodrugs derivatives, enantiomers, or polymorphs.

In some examples the compounds are prodrugs.

The term “prodrug” used herein refers to compounds which are not pharmaceutically active themselves but which are transformed into their pharmaceutical active form in vivo, for example in the subject to which the compound is administered.

In some examples, the multiple sclerosis is primary progressive multiple sclerosis.

In some example, the multiple sclerosis is secondary progressive multiple sclerosis.

In some example, the multiple sclerosis is progressive relapsing multiple sclerosis.

The compounds and/or compositions described herein may be administered either simultaneously (or substantially simultaneously) or sequentially, dependent upon the condition to be treated, and may be administered in combination with other treatment(s). The other treatment(s), may be administered either simultaneously (or substantially simultaneously) or sequentially.

In some example, the other or additional treatment further comprises administering a therapeutically effective amount of Laquinimod, Fingolimod, Masitinib, Ocrelizumab, Ibudilast, Anti-LINGO-1, MD1003 (high concentration Biotin), Natalizumab, Siponimod, Tcelna (imilecleucel-T), Simvastatin, Dimethyl fumarate, Autologous haematopoietic stem cell transplantation, Amiloride, Riluzole, Fluoxetine, Glatiramer Acetate, Interferon Beta, or a functional derivative thereof.

The actual amount(s) administered, and rate and time-course of administration, will depend on the nature and severity of progressive multiple sclerosis being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners.

The formulation(s) may conveniently be presented in unit dosage form and may be prepared by any methods well known in the art of pharmacy. Such methods include the step of bringing the active compound into association with a carrier, which may constitute one or more accessory ingredients. In general, the formulations are prepared by uniformly and intimately bringing into association the active compound with liquid carriers or finely divided solid carriers or both, and then if necessary shaping the product.

The compounds and compositions may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot/for example, subcutaneously or intramuscularly.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in- water liquid emulsion or a water-in-oil liquid emulsion; as a bolus; as an electuary; or as a paste.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection.

The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

In another aspect, there is described a method of identifying a compound for the treatment of progressive multiple sclerosis, comprising: selecting one or more compounds from a library of compounds that prevent or reduce iron-mediated neurotoxicity in vitro, selecting one or more compounds from step (b) that prevent or reduce mitochondrial damage in vitro; selecting one or more compounds from step (a) for anti-oxidative properties, selecting one or more compound from step (a) for ability to reduce T-cell proliferation in vitro, optionally, after step (a), selecting a compound from step (a) which is predicted or known to be able to cross the blood brain barrier, or having a suitable side effect profile, or having a suitable tolerability.

Methods of the invention are conveniently practiced by providing the compounds and/or compositions used in such method in the form of a kit. Such a kit preferably contains the composition. Such a kit preferably contains instructions for the use thereof.

In one example, there is described a kit for the treatment of progressive multiple sclerosis, comprising: one or more of dipyridamole, clopidogrel, cefaclor, clarithromycin, erythromycin, rifampin, loperamide, ketoconazole, labetalol, methyldopa, metoprolol, atenolol, carvedilol, indapamide, mefloquine, primaquine, mitoxanthrone, levodopa, trimeprazine, chlorpromazine, clozapine, periciazine, flunarizine, dimenhydrinate, diphenhydramine, promethazine, phenazopyridine, yohimbine, memantine, liothyronine, clomipramine, desipramine, doxepin, imipramine, trimipramine, or functional derivative thereof; and instructions for use.

In another example, the kit further comprises one or more of Laquinimod, Fingolimod, Masitinib, Ocrelizumab, Ibudilast, Anti-LINGO-1, MD1003 (high concentration Biotin), Natalizumab, Siponimod, Tcelna (imilecleucel-T), Simvastatin, Dimethyl fumarate, Autologous haematopoietic stem cell transplantation, Amiloride, Riluzole, Fluoxetine, or a functional derivative thereof; and instructions for use.

In one example there is described a pharmaceutical composition comprising clomipramine, or a functional derivative thereof, for treating progressive multiple sclerosis, primary progressive multiple sclerosis, secondary progressive multiple sclerosis, or progressive relapsing multiple sclerosis.

In one aspect there is described a kit for the treatment of progressive multiple sclerosis comprising: a therapeutically effective amount of indapamide, or a functional derivative thereof, and instructions for use.

In one aspect there is described a kit for the treatment of progressive multiple sclerosis comprising: a therapeutically effective amount of indapamide, or a functional derivative thereof, and one or more of hydroxychloroquine, minocycline, or clomipramine; and instructions for use.

A kit may also include one or more of a container, a buffer, a diluent, a filter, a needle, or a syringe.

To gain a better understanding of the invention described herein, the following examples are set forth. It should be understood that these example are for illustrative purposes only. Therefore, they should not limit the scope of this invention in any way.

### EXAMPLES

In the following examples, standard methodologies were employed, as would be appreciated by the skilled worker.

Materials and Methods

Cell Culture and Treatment of Human Neurons

Human neurons were isolated from brain tissues of therapeutically aborted 15-20 week old fetuses, in accordance with ethics approval of the University of Calgary ethics committee, after written informed consent of the pregnant donors. Neurons were isolated as previously described (Vecil et al., 2000) brain specimens were washed in phosphate buffered saline (PBS) to remove blood, followed by removal of meninges. Tissue was mechanically dissected, followed by digestion in DNase (6-8 ml of 1 mg/ml; Roche), 4 ml 2.5% trypsin and 40 ml PBS (37° C., 25 min). Thereafter, the digestion was stopped by addition of 4 ml fetal calf serum (FCS). The solution was filtered through a 132 μm filter and centrifuged (three times, 1,200 rpm, 10 min). Cells were cultured in feeding medium of minimal essential medium (MEM) supplemented with 10% fetal bovine serum (FBS), 1 μM sodium pyruvate, 10 μM glutamine, 1× non-essential amino acids, 0.1% dextrose and 1% penicillin/streptomycin (all culture supplements from Invitrogen, Burlington, Canada). The initial isolates of mixed CNS cell types were plated in poly-L-ornithine coated (10 μg/ml) T75 flasks and cultured for at least two cycles (Vecil et al., 2000) in medium containing 25 μM cytosine arabinoside (Sigma-Aldrich, Oakville, Canada) to inhibit astrocyte proliferation and to deplete this major contaminating cell type. For experiments, the neuron-enriched cultures were retrypsinized and cells were plated in poly-L-ornithine pre-coated 96-well plates at a density of 100,000 cells/well in 100 μl of the complete medium supplemented with cytosine arabinoside. Medium was changed to AIM V® Serum Free Medium (Invitrogen) after 24 h. After a period of 1 h, respective drugs were added in a concentration of 10 μM, followed by application of FeSO₄ after 1 h or 24 h, or the other toxins after 1 h. All conditions were performed in quadruplicates. A day later cells were fixed using 4% paraformaldehyde (PFA) and stored in PBS in 4° C.

We note that in tissue culture, the toxicity of iron to neurons begins immediately. Thus, it has been our experience that pretreatment with test protective agents is necessary. With the continuous insult that occurs in multiple sclerosis, a pretreatment paradigm with test compounds against iron neurotoxicity in our experiments can be justified as that simulates the protection against the next injury in the disease.

Drugs tested were contained within the 1040-compound NINDS Custom Collection II, which was purchased from Microsource Discovery (Gaylordsville, Conn., USA) and used as previously described (Samanani et al., CNS&neurological disorders drug targets 12: 741-749, 2013). Briefly, there were 80 compounds located in specific wells on each plate (e. g. B07). 3607 would thus refer to position B07 of plate 3. Each compound was supplied at a concentration of 10 mM dissolved in DMSO.

The iron stock solution was prepared using 27.8 mg iron(II) sulfate heptahydrate (FeSO₄) (Sigma-Aldrich, Oakville, Canada), 10 μl of 17.8M sulfuric acid and 10 ml deionized distilled water. After filtering with a 0.2 μm filter, FeSO₄ was added to cells in a final concentration of 25-50 μM in a volume of 50 μl medium to the cells. Rotenone was dissolved in dimethyl sulfoxide (DMSO) and used in a final concentration of 10 μM.

Hydroxyl Radical Antioxidant Capacity (HORAC) Assay

Selected compounds that prevented iron mediated neurotoxicity were analyzed for their antioxidative properties using the hydroxyl radical antioxidant capacity (HORAC) assay, in accordance with the procedure outlined in Číž et al. 2010 (Food Control 21:518-523, 2010). In this assay, hydroxyl radicals generated by a Co(II)-mediated Fenton-like reaction oxidize fluorescein causing loss of fluorescence (Ou et al., J Argricultural Food Chemistry 50:2772-2777, 2002). The presence of an anti-oxidant reduces the loss of fluorescence and this can be monitored every 5 min over a period of 60 min with a Spectra Max Gemini XS plate reader (Molecular Devices, Sunnyvale, Calif., USA) and the software SoftMax Pro version 5. For monitoring fluorescence, we used an excitation wavelength of λ=485 nm and an emission wavelength of λ=520 nm.

Proliferation of T-Lymphocytes

A previously published protocol was used for isolating and activating T-cells (Keough et al., Nature Comm 7:11312, 2016). Spleens from female C57B16 mice were harvested and after mechanical dissociation the cell suspension was passed through a 70 μm cell strainer and separated by Ficoll gradient (1800 RPM, 30 min). Splenocytes were plated (2.5×10⁵ cells in 100 μl/well) in anti-CD3 antibody coated 96-well plates (1,000 ng m1⁻¹ plate-bound anti-CD3 and 1,000 ng m1⁻¹ anti-CD28 suspended in media) to activate T-cells. Directly before plating, wells were treated with respective drugs in a final concentration of 10 μM. Cells were cultured in RPMI 1640 medium, supplemented with 10% FBS, 1 μM sodium pyruvate, 2 mM L-alanyl-L-glutamine, 1% penicillin/streptomycin, 1% HEPES and 0.05 mM 2-mercaptoethanol (all supplements were from Invitrogen). After 48 h, ³H-thymidine was added in a concentration of 1 μCi per well, and cells were harvested after 24 h on filter mats. Mats were then evaluated for radioactivity (counts per minute) using a liquid scintillation counter.

Activity on B-Lymphocytes

Venous blood from healthy volunteers was obtained and peripheral blood mononuclear cells (PBMCs) were isolated by Ficoll gradient centrifugation (1800 RPM, 30 min). From PBMCs, B-cells were isolated by positive selection with CD19 directed microbeads (Stemcell Technologies). Purity was assessed by FACS after staining for CD19 (Stemcell Technologies). Cells were plated in a concentration of 2.5×10⁵ cells/well in X-VIVO™ medium (Lonza) supplemented with 1% penicillin/streptomycin and 1% Glutamax and treated with drugs for 1 h. Cells were then activated with 10 μg/ml IgM BCR cross-linking antibody (XAb) (Jackson ImmunoResearch), 1 μg/mlanti-CD4OL and IL-4 20 ng/ml for 24 h as previously described (Li et al., Science Translational Med 7:310ra166, 2015). Conditioned media were harvested after 24 h for ELISA. Medium as well as respective drugs were re-added followed by application of ³H-thymidine in a concentration of 1 μCi per well to investigate proliferation. After 24 h, cells were harvested on filter mats and after drying counts per minutes were measured using a liquid scintillation counter.

### Flow Cytometry

Two days after activation and drug treatment splenocytes were harvested, washed with PBS followed by resuspension in PBS with 2% FBS. Cell cycle analysis was performed taking advantage of propidium iodide staining (50 μg/ml) using an established protocol (Besson and Yong, 2000). Cells were washed in cold PBS and resuspended in PI/Triton X-100 staining solution (10 ml 0.1% (v/v) Triton X-100 in PBS with 2 mg DNAse-free RNAse A and 0.4 ml of 500 μg/ml PI), followed by incubation at 4° C. for 30 min. Stained cells were analyzed on a FACSCalibur™ with the software CellQuest™ (BD Biosciences). Cell cycle analysis was conducted using the software ModFit LT, version 3.3 (Verity Software House Inc.).

FACS Gating Strategy

Cells were identified by gating into the lymphocyte population, followed by single cell gating to exclude doublets and aggregates. This was followed by identification of the G0/G1 population and processing with the software ModFit LT, version 3.3 (Verity Software House Inc.) to calculate the percentage of cells in different cell cycles.

Intracellular staining was performed following fixation and permeabilization of splenocytes using the Fixation/Permeabilization Solution Kit (BD Biosciences, Mississauga, Canada), followed by staining with anti-human/mouse phospho-AKT (S473) APC antibody, anti-human/mouse phospho-mTOR (S2448) PE-Cyanine7 antibody and anti-human/mouse phospho-ERK1/2 (T202/Y204) PE antibody (all eBioscience, San Diego, Calif.). Stained cells were analyzed on a FACSCalibur™ with the software CellQuest™ (BD Biosciences).

Immunocytochemistry and Microscopy

Staining was performed at room temperature. A blocking buffer was first introduced for 1 h followed by incubation with primary antibody overnight in 4° C. Neurons were stained using mouse anti-microtubule-associated protein-2 (MAP-2) antibody, clone HM-2 (dilution 1:1,000; Sigma-Aldrich, Oakville, Canada). (Table 3)

TABLE 3

| Antibody | Company | Catalog | Species | Dilation |
|---|---|---|---|---|
| Iba1 | Wako | 019-18741 | Rabbit | 1:250 |
| MAP-2, clone HM-2 | Sigma-Aldrich | M4403 | Mouse | 1:1,000 |

Primary antibody was visualized with Alexa Fluor 488 or 546-conjugated secondary antibody (dilution 1:250, Invitrogen, Burlington, Canada). Cell nuclei were stained with Hoechst S769121 (nuclear yellow). Cells were stored in 4° C. in the dark before imaging.

Images were taken using the automated ImageXpress® imaging system (Molecular Devices, Sunnyvale, Calif.) through a 10x objective microscope lens, displaying 4 or 9 sites per well. Images were analyzed with the software MetaXpress® (Molecular Devices, Sunnyvale, Calif.) using the algorithm “multiwavelength cell scoring” (Lau et al., Ann Neurol 72:419-432, 2012). Cells were defined according to fluorescence intensity and size at different wavelengths. Data from all sites per well were averaged to one data point.

Live Cell Imaging

Neurons were prepared as described above. Directly after the addition of FeSO₄ to healthy neurons, the live cell-permeant Hoechst 33342 (1:2 diluted in AIM-V medium, nuclear blue; ThermoFisher Scientific, Grand Island, NY, USA) and the live cell-impermeable propinium iodide (PI, 1:20 diluted in AIM-V medium) were added in a volume of 20 μI (Sigma-Aldrich). In compromised cells, PI could now diffuse across the plasma membrane. Live cell imaging was performed using the automated ImageXpress® imaging system under controlled environmental conditions (37° C. and 5% CO2). Images were taken from 9 sites per well at baseline and then every 30 min for 12h. After export with MetaXpress®, videos were edited with ImageJ (NIH) in a uniform manner. Nuclei were pseudo colored in cyan, PI-positive cells in red.

Experimental Autoimmune Encephalomyelitis (EAE)

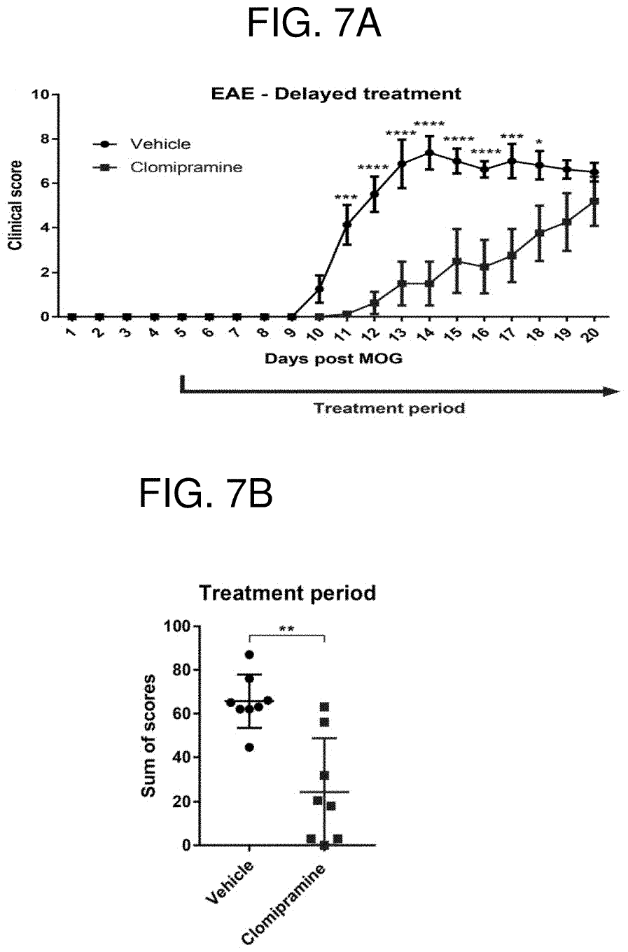

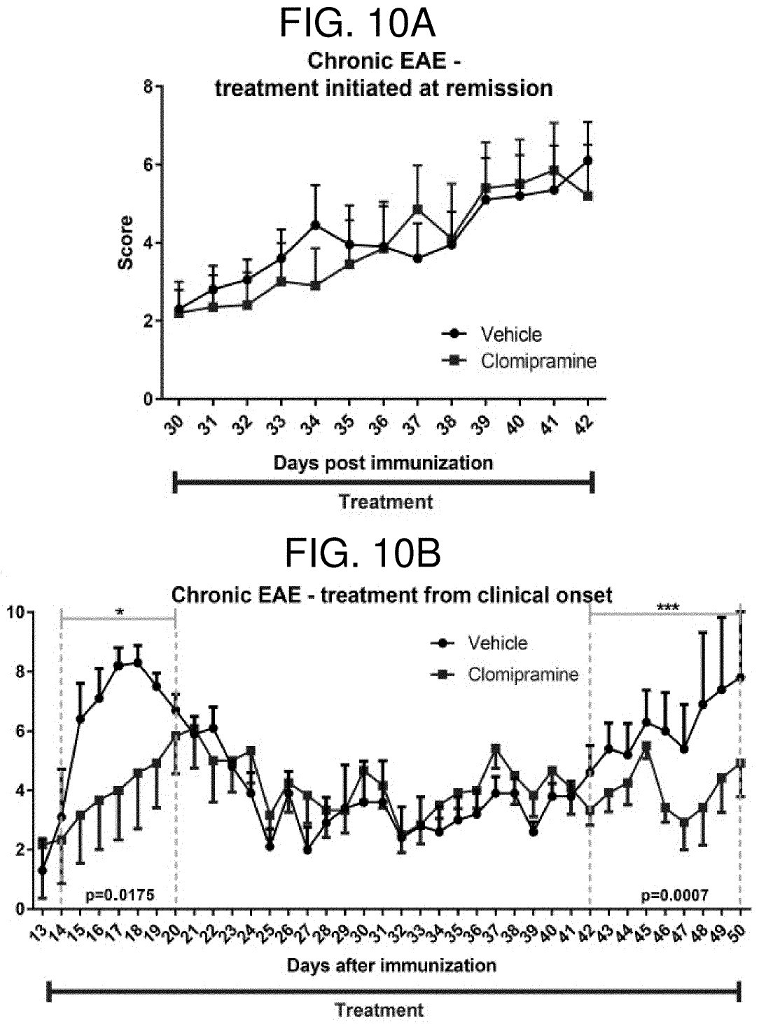

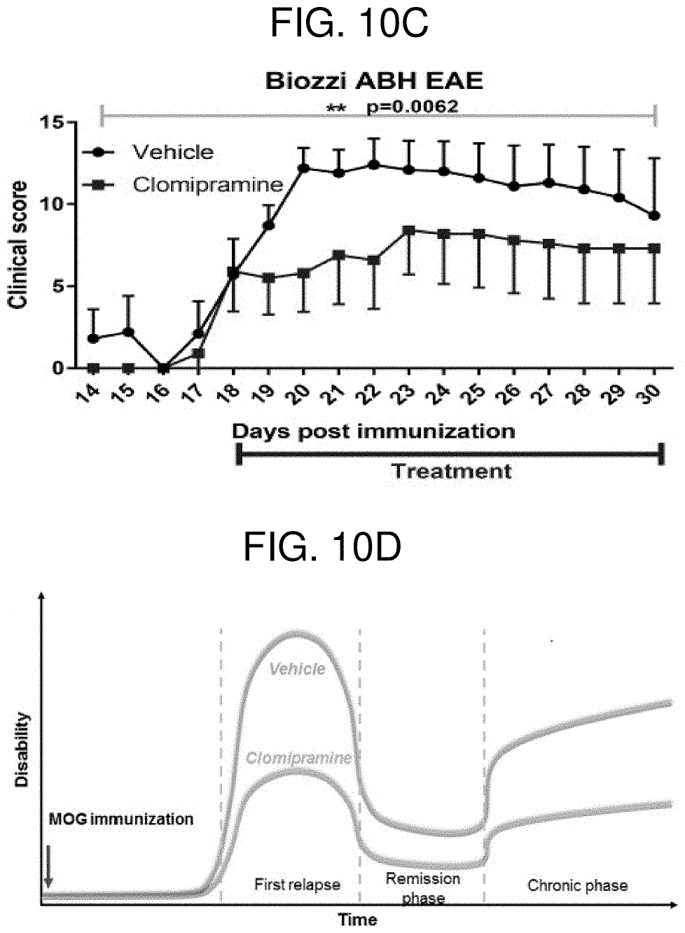

EAE was induced in 8-10 week-old female C57BL/6 mice (Charles River, Montreal, Canada). Mice were injected with 50 g of MOG₃₅₋₅₅ (synthesized by the Peptide Facility of the University of Calgary) in Complete Freund's Adjuvant (Thermo Fisher Scientific) supplemented with 10 mg/ml Mycobacterium tuberculosis subcutaneously on both hind flanks on day 0. In addition, pertussis toxin (0.1 μg/200 μl; List biological Laboratories, Hornby, Canada) was injected intraperitoneal (IP) on days 0 and 2. Animals were treated with clomipramine (25 mg/kg; 100 μI of 5 mg/ml solution) by IP injection by IP injection from day 0 or day 5 (FIG. 7,8), from day 30 at remission (FIG. 10a), or from 13 at onset of clinical signs (FIG. 10b). The solution of clomipramine was prepared daily in fresh PBS.

The Biozzi ABH mouse model (Al-lzki et al., Multiple Sclerosis 17:939-948, 2011) was used as a model of progression. EAE was induced in Biozzi ABH mice aged 8-10 weeks by the subcutaneous application of 150 μl emulsion in both sides of the hind flanks. The emulsion was prepared as follows: Stock A consisted of 4 ml of incomplete Freund's adjuvant mixed with 16 mg M. tuberculosis and 2 mg M. butyricum. One ml of stock A was mixed with 11.5 ml incomplete Freund's adjuvant to become stock B. Stock B was mixed in equal volume with spinal cord homogenate (SCH) in PBS before injection. SCH was used in a concentration of 6.6 mg/ml emulsion each for 2 injections (days 0 and 7).

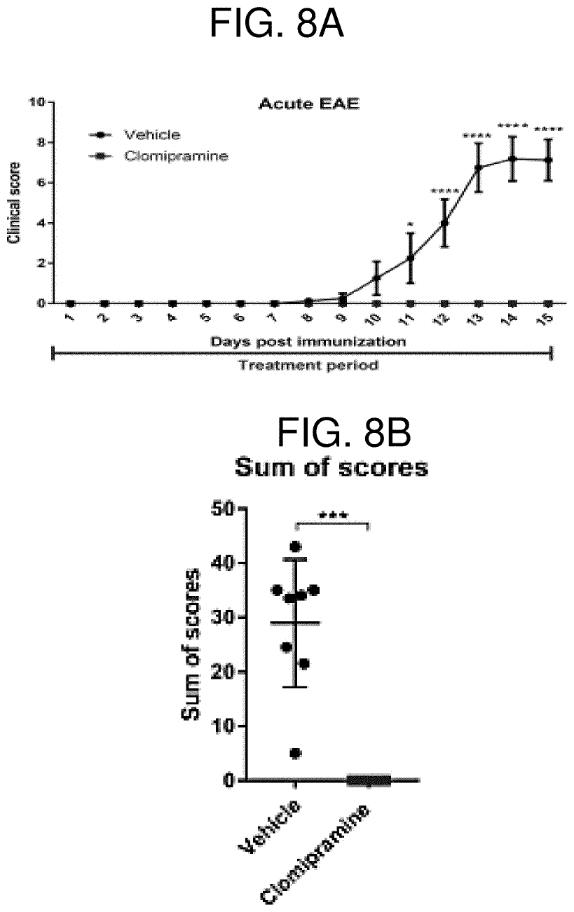

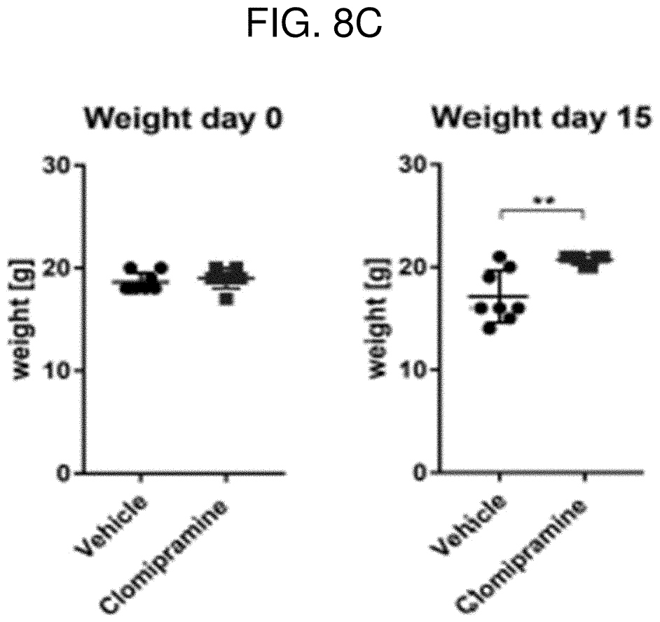

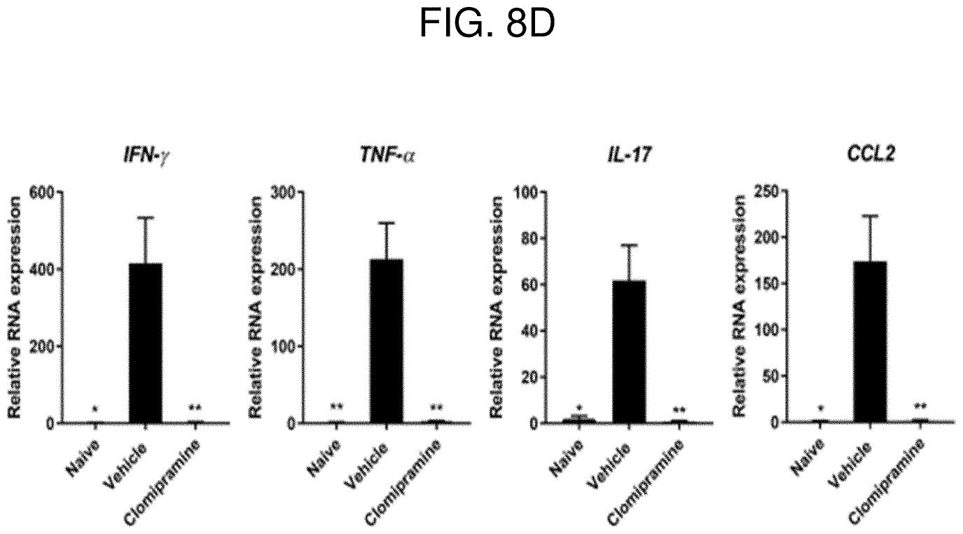

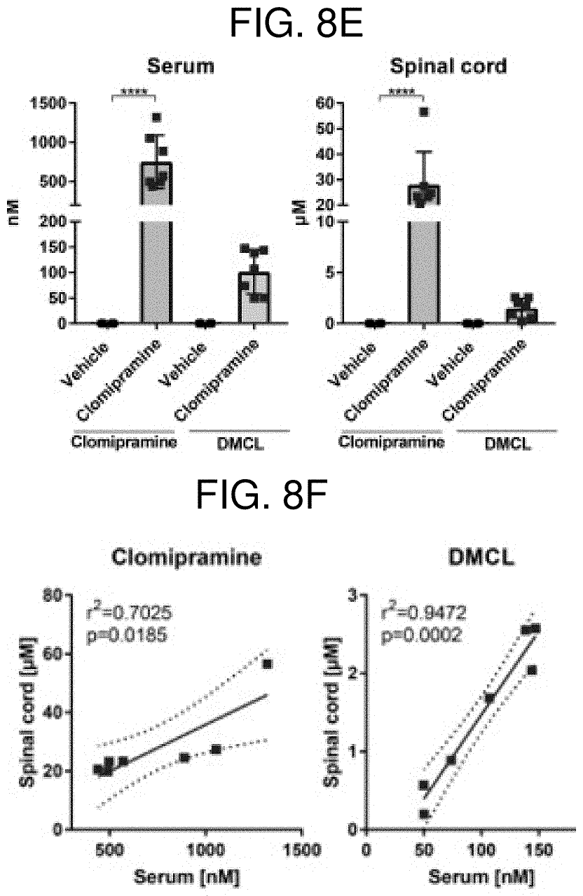

The number of animals was chosen according to experience with previous experiments (FIG. 7: 8/8 (vehicle/clomipramine); FIG. 8: 8/7; FIG. 10 a) 10/10; b) 5/6; c) 5/5), and animals were randomized after induction of EAE. Animals were handled according to the Canadian Council for Animal Care and the guidelines of the animal facility of the University of Calgary. All animal experiments received ethics approval (AC12-0181) from the University of Calgary's Animal Ethics Committee. Mice were scored daily using a 15-point scoring system, the investigator was not blinded (Giuliani F, Fu SA, Metz LM, Yong VW. Effective combination of minocycline and interferon-beta in a model of multiple sclerosis. Journal of neuroimmunology 165, 83-91 (2005)).

Histological Analyses

One h after the last administration of clomipramine animals were anesthetized with ketamine/xylazine, blood was taken by an intracardiac puncture for serum, and animals were then subjected to PBS-perfusion. Spinal cords and cerebella were removed. The thoracic cords were fixed in 10% buffered formalin, followed by embedding in paraffin. Cervical and lumbar cords were snap frozen. Tissue was further processed as previously described 52. Briefly, the thoracic spinal cord was cut longitudinally from the ventral to the dorsal side with sections of 6 μm thickness. Sections were stained with hematoxylin/eosin, lba1 to visualize microglia and Bielschowsky's silver stain to visualize axons. Sections for lba1 and Bielschowsky's silver stain were blinded, before images depicting area of maximal microglial activation or axonal damage were chosen for blinded rank order analysis by a second investigator.

PCR

Lumbar spinal cords were harvested, snap frozen in liquid nitrogen and stored in -80° C. Samples were homogenized in 1 ml Trizol followed by the addition of 200 μI chloroform. The suspension was shaken, centrifuged (11,500 RPM for 15 min at 4° C.) and the RNA-containing upper phase was transferred into a new tube and precipitated with equal amounts of 70% ethanol. RNA was extracted using the RNeasy Mini Kit according to the manufacturer's instruction (Qiagen). RNA concentrations were measured using a Nanodrop (Thermo Fisher Scientific). cDNA preparation was performed using the RT2 First Strand kit (Qiagen) with 1μg of RNA according to the manufacturer's instructions. Real time PCR was performed using the QuantStudio 6 Flex (Applied Biosystems by Life Technologies) with FAST SYBR Green and primers for Gapdh (Qiagen) as housekeeping gene, lfn-γ (Qiagen, QT01038821), Tnfa (Qiagen, QT00104006), II-17 (SABiosciences, PPM03023A-200) and CcI2 (Qiagen, QT00167832). Relative expression was calculated using the ΔΔCT method with Gapdh as housekeeping gene. Data were normalized to gene expression in naïve mice.

Liquid Chromatography-Mass Spectrometry

The assay is a modification of the liquid chromatography-mass spectrometry (LC-MS) assay of Shinokuzack et al. (Forensic Science International 62:108-112, 2006). For preparation of samples, 100 pl of ice cold methanol were added to 100 pl of serum in each sample after addition of the internal standard maprotiline. The tubes were vortexed and left on ice for 10 min followed by centrifugation at 10,000×g for 4 min. An equal amount of distilled water was added to each supernatant. Spinal cord samples were each homogenized in 10 volumes of ice-cold 80% methanol. Twenty pl of o-phosphoric acid were added to all samples after addition of internal standard (maprotiline). The tubes were vortexed and left on ice for 10 min, followed by centrifugation at 10,000×g for 4 min and an equal volume of distilled water was added to each supernatant.

An HLB Prime pelution plate was employed for sample cleanup for both serum and spinal cord samples. After running the supernatants described above through the wells, all wells were washed with 5% methanol in water and allowed to dry completely before elution with 100 μl 0.05% formic acid in methanol:acetonitrile (1:1). The eluents were transferred to low volume μl glass inserts (Waters, Milford, Mass., USA) and 10 μl from each eluent were injected into the LC-MS system.

Analysis was performed using a Waters ZQ Mass detector fitted with an ESCI Multi-Mode ionization source and coupled to a Waters 2695 Separations module (Waters). Mass Lynx 4.0 software was used for instrument control, data acquisition and processing. HPLC separation was performed on an Atlantis dC18 (3 μm, 3.0×100 mm) column (Waters) with a guard column of similar material. Mobile phase A consisted of 0.05% formic acid in water and mobile phase B was composed of 0.05% formic acid in acetonitrile. Initial conditions were 80% A and 20% B at a flow rate of 0.3 mL/min. A gradient was run, increasing to 80% B in 15 min; this was followed by a return to initial conditions. The column heater and sample cooler were held at 30° C. and 4° C. respectively. Optimized positive electrospray parameters were as follows: Capillary voltage 3.77 kV; Rf lens voltage 1.2 V; source 110° C.; desolvation temperature 300° C.; cone gas flow (nitrogen) 80 L/h; desolvation gas flow (nitrogen) 300 L/h. Cone voltage was varied for each compound: clomipramine 25 V; N-desmethylclomipramine 22 V; and maprotiline 25 V. The m/z ratios for clomipramine, N-desmethylclomipramine and maprotiline (internal standard) were 315, 301 and 278 respectively.

Calibration curves consisting of varying amounts of authentic clomipramine and N-desmethylclomipramine and the same fixed amount of maprotiline as added to the samples being analyzed were run in parallel through the procedure described above and the ratios of clomipramine and N-desmethylclomipramine to maprotiline were used to determine the amount of drug and metabolite in the serum and spinal cord samples.

Statistical Analysis

Statistical analysis was performed using the Graphpad Prism software version 7 (La Jolla, Calif., USA). For cell culture experiments, one-way ANOVA with different post-hoc analyses was applied, as stated in the respective figure legends. EAE scores were analyzed using two-way ANOVA with Sidak's multiple comparison as post-hoc analysis. Statistical significance was considered as p<0.05 (*), p<0.01 (**), p<0.001 (***) and p<0.0001 (****). All experiments were performed in quadruplicates, if not otherwise specified.

Results

Protection Against Iron and Rotenone Neurotoxicity

Of the 1040 compounds available in the NINDS Custom Collection II, we first conducted a search of available information to exclude those that were either experimental, agricultural, not available as oral drug, not listed at Health Canada, steroid hormones or veterinary medications. Moreover, we omitted those that were not known to cross the blood-brain barrier. We note that while we selected drugs that are orally available, for ease of use, this does not imply that injectable medications would not be effective medications in progressive multiple sclerosis, as illustrated by ocrelizumab recently (Montalban X, et al. Ocrelizumab versus Placebo in Primary Progressive Multiple Sclerosis. N Engl J Med 376, 209-220 (2017). Out of the original list, 791 compounds were thus excluded and 249 were selected for further testing. The detailed information of each of the 249 compounds are provided in Table 1

TABLE 1

| ID | MOLENAME | plate | position | cas# | FORMULA | MolWt |
|---|---|---|---|---|---|---|
| 01502057 | 5-CHLOROINDOLE- | 402006 | D09 | | C9H6ClNO2 | 195.61 |
| | 2-CARBOXYLIC ACID | | | | | |
| 01500665 | ACEBUTOLOL | 402011 | A11 | 34381-68-5, | C18H29ClN2O4 | 372.90 |
| | HYDROCHLORIDE | | | 37517-30-9 | | |
| | | | | [acebutolol] | | |
| 01500101 | ACETAMINOPHEN | 402001 | E04 | 103-90-2 | C8H9NO2 | 151.17 |
| 01500102 | ACETAZOLAMIDE | 402001 | B02 | 59-66-5 | C4H6N4O3S2 | 222.25 |
| 01500105 | ACETYLCYSTEINE | 402001 | D08 | 616-91-1 | C5H9NO3S | 163.20 |
| 01503603 | ACYCLOVIR | 402008 | C03 | 59277-89-3 | C8H11N5O3 | 225.21 |
| 01500108 | ALLOPURINOL | 402001 | F11 | 315-30-0 | C5H4N4O | 136.11 |
| 01505204 | ALMOTRIPTAN | 402009 | A06 | 154323-57-6 | C17H25N3O2S | 335.47 |
| 01503065 | ALTRETAMINE | 402007 | C07 | 645-05-6 | C9H18N6 | 210.28 |
| 01500110 | AMANTADINE | 402001 | H02 | 665-66-7, 768-94-5 | C10H18ClN | 187.71 |
| | HYDROCHLORIDE | | | [amantadine] | | |
| 01500111 | AMIKACIN | 402001 | A03 | 39831-55-5, | C22H47N5O21S2 | 781.77 |
| | SULFATE | | | 37517-28-5 | | |
| | | | | [amikacin] | | |
| 01500112 | AMILORIDE | 402001 | B03 | 17440-83-4, | C6H9Cl2N7O | 266.09 |
| | HYDROCHLORIDE | | | 2016-88-8 | | |
| | | | | [anhydrous], | | |
| | | | | 2609-46-3 | | |
| | | | | [amiloride] | | |
| 02300165 | AMIODARONE | 402013 | B04 | 1951-25-3 | C25H30ClI2NO3 | 681.78 |
| | HYDROCHLORIDE | | | | | |
| 01500117 | AMITRIPTYLINE | 402001 | G03 | 549-18-8, 50-48-6 | C20H24ClN | 313.87 |
| | HYDROCHLORIDE | | | [amitriptyline] | | |
| 01505202 | AMLODIPINE | 402009 | G05 | 111470-99-6 | C26H31ClN2O8S | 567.06 |
| | BESYLATE | | | | | |
| 01500120 | AMOXICILLIN | 402001 | D02 | 61336-70-7, | C16H19N3O5S | 365.41 |
| | | | | 26787-78-0 | | |
| | | | | [anhydrous] | | |
| 01500122 | AMPHOTERICIN B | 402001 | B04 | 1397-89-3 | C47H73NO17 | 924.10 |
| 01500128 | ANTIPYRINE | 402001 | F04 | 60-80-0 | C11H12N2O | 188.23 |
| 01500130 | ASPIRIN | 402013 | D06 | 50-78-2 | C9H8O4 | 180.16 |
| 01501127 | ATENOLOL | 402006 | C02 | 29122-68-7 | C14H22N2O3 | 266.34 |
| 01503722 | ATORVASTATIN | 402008 | H05 | 134523-03-8, | C33H33CaFNO5 | 582.71 |
| | CALCIUM | | | 134523-00-5 | | |
| | | | | [atorvastatin] | | |
| 01504210 | ATOVAQUONE | 402008 | F11 | 95233-18-4 | C22H19ClO3 | 366.85 |
| 01500133 | AZATHIOPRINE | 402001 | A05 | 446-86-6 | C9H7N7O2S | 277.27 |
| 01503679 | AZITHROMYCIN | 402008 | B05 | 83905-01-5, | C38H72N2O12 | 749.00 |
| | | | | 117772-70-0 | | |
| | | | | [dihydrate] | | |
| 01500134 | BACITRACIN | 402001 | B05 | 1405-87-4 | C66H103N17O16S | 1422.73 |
| 01500135 | BACLOFEN | 402001 | C05 | 1134-47-0 | C10H12ClNO2 | 213.67 |
| 01505200 | BENAZEPRIL | 402009 | E05 | 86541-74-4 | C24H29ClN2O5 | 460.96 |
| | HYDROCHLORIDE | | | | | |
| 01500137 | BENSERAZIDE | 402001 | D0S | 322-35-0 | C10H16ClN3O5 | 293.71 |
| | HYDROCHLORIDE | | | | | |
| 01500142 | BENZTROPINE | 402001 | H05 | 132-17-2, 86-13-5 | C21H27NO5S | 405.52 |
| | | | | [benztropine] | | |
| 01500146 | BETHANECHOL | 402001 | A11 | 590-63-6, 674-38-4 | C7H17ClN2O2 | 196.68 |
| | CHLORIDE | | | [bethanechol] | | |
| 01502046 | BEZAFIBRATE | 402006 | A09 | 41859-67-0 | C19H20ClNO4 | 361.83 |
| 01500147 | BISACODYL | 402001 | D06 | 603-50-9 | C22H19NO4 | 361.40 |
| 01503985 | BROMPHENIRAMINE | 402012 | D11 | 980-71-2, 86-22-6 | C20H23BrN2O4 | 435.32 |
| | MALEATE | | | [brompheniramine] | | |
| 01500813 | BUDESONIDE | 402011 | F03 | 51333-22-3 | C25H34O6 | 430.55 |
| | | | | [11(gr b), 16(gr a)] | | |
| | | | | 51372-29-3 | | |
| | | | | [(11(gr b), 16(gr a) | | |
| | | | | [//R//])] | | |
| | | | | 51372-28-2 | | |
| | | | | [(11(gr b), 16(gr a)[//S//])] | | |
| 01502004 | BUMETANIDE | 402006 | F06 | 28395-03-1 | C17H20N2O5S | 364.42 |
| 01504174 | BUPROPION | 402008 | G10 | 31677-93-7, | C13H19Cl2NO | 276.21 |
| | | | | 34911-55-2 | | |
| | | | | [bupropion] | | |
| 01500152 | BUSULFAN | 402001 | F06 | 55-98-1 | C6H14O6S2 | 246.30 |
| 01504261 | CANDESARTAN | 402009 | B04 | 139481-59-7 | C33H34N6O6 | 610.68 |
| | CILEXTIL | | | | | |
| 01500682 | CAPTOPRIL | 402005 | F03 | 62571-86-2 | C9H15NO3S | 217.29 |
| 01500158 | CARBACHOL | 402001 | B07 | 51-83-2 | C6H15ClN2O2 | 182.65 |
| 01500159 | CARBAMAZEPINE | 402001 | C07 | 298-46-4 | C15H12N2O | 236.28 |
| 01504257 | CARVEDILOL | 402009 | F03 | 72956-09-3 | C28H32N2O10 | 556.57 |
| | TARTRATE | | | (carvedilol) | | |
| 01500771 | CEFACLOR | 402005 | D06 | 70356-03-5, | C15H14ClN3O4S | 367.81 |
| | | | | 53994-73-3 | | |
| | | | | [anhydrous] | | |
| 01500163 | CEFADROXIL | 402001 | G07 | 66592-87-8, | C16H17N3O5S | 363.39 |
| | | | | 50370-12-2 | | |
| | | | | [anhydrous], | | |
| | | | | 119922-89-9 | | |
| | | | | [hemihydrate] | | |
| 01502028 | CEPHALEXIN | 402012 | H11 | 23325-78-2, | C16H17N3O4S | 347.40 |
| | | | | 15686-71-2 | | |
| | | | | [anhydrous] | | |
| 01500183 | CHLORPHENIRAMINE | 402001 | D09 | 113-92-8, 132-22-9 | C20H23ClN2O4 | 390.87 |
| | (S) MALEATE | | | [chlorpheniramine] | | |
| 01500184 | CHLORPROMAZINE | 402001 | E09 | 50-53-3 | C17H19ClN2S | 318.87 |
| 01500185 | CHLORPROPAMIDE | 402001 | F09 | 94-20-2 | C10H13ClN2O3S | 276.74 |
| 01500187 | CHLORTHALIDONE | 402001 | A07 | 77-36-1 | C14H11ClN2O4S | 338.77 |
| 01500684 | CIMETIDINE | 402005 | G03 | 51481-61-9 | C10H16N6S | 252.34 |
| 01503614 | CIPROFLOXACIN | 402008 | E03 | 85721-33-1 | C17H18FN3O3 | 331.35 |
| 01504231 | CLARITHROMYCIN | 402009 | H02 | 81103-11-9 | C38H69NO13 | 747.97 |
| 01500191 | CLEMASTINE | 402001 | D10 | 15686-51-8 | C25H30ClNO5 | 459.97 |
| 01500193 | CLINDAMYCIN | 402001 | F10 | 21462-39-5, | C18H34Cl2N2O5S | 461.45 |
| | HYDROCHLORIDE | | | 58207-19-5 | | |
| | | | | [monohydrate], | | |
| | | | | 18323-44-9 | | |
| | | | | [clindamycin] | | |
| 02300061 | CLOMIPRAMINE | 402012 | G02 | 17321-77-6, | C19H24Cl2N2 | 351.32 |
| | HYDROCHLORIDE | | | 303-49-1 | | |
| | | | | [clomipramine] | | |
| 01500198 | CLONIDINE | 402001 | C06 | 4205-91-8, | C9H10Cl3N3 | 266.56 |
| | HYDROCHLORIDE | | | 4205-90-7 | | |
| | | | | [clonidine] | | |
| 01503710 | CLOPIDOGREL | 402008 | E05 | 113665-84-2 | C16H18ClNO652 | 419.91 |
| | SULFATE | | | | | |
| 01500200 | CLOTRIMAZOLE | 402013 | H06 | 23593-75-1 | C22H17ClN2 | 344.85 |
| 01500201 | CLOXACILLIN | 402001 | B11 | 7081-44-9, | C19H17ClN3NaO5S | 457.87 |
| | SODIUM | | | 642-78-4 | | |
| | | | | [anhydrous] | | |
| 01500685 | CLOZAPINE | 402005 | H03 | 5786-21-0 | C18H19ClN4 | 326.83 |
| 01500205 | COLCHICINE | 402001 | D11 | 64-86-8 | C22H25NO6 | 399.45 |
| 01500209 | CRESOL | 402001 | H11 | 1319-77-3 | C7H8O | 108.14 |
| 01500210 | CROMOLYN | 402002 | A02 | 15826-37-6, | C23H14Na2O11 | 512.34 |
| | SODIUM | | | 16110-51-3 | | |
| | | | | [cromolyn] | | |
| 01503207 | CYCLOBENZAPRINE | 402011 | H08 | 6202-23-9, | C20H22ClN | 311.86 |
| | HYDROCHLORIDE | | | 303-53-7 | | |
| | | | | [cyclobenzaprine] | | |
| 01500213 | CYCLOPHOSPHAMIDE | 402002 | D02 | 6055-19-2, 50-18-0 | C7H17Cl2N2O3P | 279.10 |
| | HYDRATE | | | [anhydrous] | | |
| 01502202 | CYCLOSPORINE | 402007 | B03 | 59865-13-3 | C62H111N11O12 | 1202.64 |
| 01500220 | DANAZOL | 402002 | G02 | 17230-88-5 | C22H27NO2 | 337.47 |
| 01500222 | DAPSONE | 402002 | H02 | 80-08-0 | C12H12N2O2S | 248.31 |
| 01503127 | DEQUALINIUM | 402007 | A09 | 522-51-0, | C30H40Cl2N4 | 527.59 |
| | CHLORIDE | | | 6707-58-0 | | |
| | | | | [dequalinium] | | |
| 01500227 | DESIPRAMINE | 402002 | D03 | 58-28-6, 50-47-5 | C18H23ClN2 | 302.85 |
| | HYDROCHLORIDE | | | [desipramine] | | |
| 01500233 | DEXTROMETHORP | 402002 | G03 | 6700-34-1, | C18H26BrNO | 352.32 |
| | HAN | | | 125-69-9 | | |
| | HYDROBROMIDE | | | [anhydrous], | | |
| | | | | 125-71-3 | | |
| | | | | [dextromethor | | |
| | | | | phan] | | |
| 02300206 | DIAZOXIDE | 402013 | A02 | 364-98-7 | C8H7ClN2O2S | 230.67 |
| 01500237 | DICLOFENAC SODIUM | 402002 | B04 | 15307-79-6 | C14H10Cl2NNaO2 | 318.14 |
| 01500245 | DIFLUNISAL | 402002 | G04 | 22494-42-4 | C13H8F2O3 | 250.20 |
| 01500247 | DIGOXIN | 402002 | H04 | 20830-75-5 | C41H64O14 | 780.96 |
| 02300214 | DILTIAZEM | 402012 | G11 | 33286-22-5, | C22H27ClN2O4S | 450.99 |
| | HYDROCHLORIDE | | | 42399-41-7 | | |
| | | | | [diltiazem] | | |
| 01500251 | DIMENHYDRINATE | 402002 | B05 | 523-87-5 | C24H28ClN5O3 | 469.98 |
| 01500256 | DIPHENHYDRAMINE | 402002 | D0S | 147-24-0 | C17H22ClNO | 291.82 |
| | HYDROCHLORIDE | | | | | |
| 01500258 | DIPHENYLPYRALINE | 402002 | E05 | 132-18-3 147-20-6 | C19H24ClNO | 317.86 |
| | HYDROCHLORIDE | | | [diphenylpyraline] | | |
| 01500259 | DIPYRIDAMOLE | 402002 | F05 | 58-32-2 | C24H40N8O4 | 504.64 |
| 01500261 | DISOPYRAMIDE | 402002 | H05 | 3737-09-5 | C21H32N3O5P | 437.48 |
| | PHOSPHATE | | | | | |
| 01500264 | DOXEPIN | 402013 | F09 | 1229-29-4, | C19H22ClNO | 315.85 |
| | HYDROCHLORIDE | | | 1668-19-5 | | |
| | | | | [doxepin], | | |
| | | | | 4698-39-9 | | |
| | | | | [(//E//)-isomer], | | |
| | | | | 25127-31-5 | | |
| | | | | [(//Z//)-isomer] | | |
| 01500266 | DOXYCYCLINE | 402011 | F09 | 17086-28-1, | C22H25ClN2O8 | 480.91 |
| | HYDROCHLORIDE | | | 564-25-0 | | |
| | | | | [anhydrous] | | |
| 01500267 | DOXYLAMINE | 402013 | G08 | 562-10-7, 469-21-6 | C21H28N2O5 | 388.47 |
| | SUCCINATE | | | [doxylamine] | | |
| 02300219 | EDROPHONIUM | 402010 | H07 | 116-38-1, 312-48-1 | C1OH16ClNO | 201.70 |
| | CHLORIDE | | | [edrophonium] | | |
| 01501214 | ENALAPRIL | 402011 | B05 | 76095-16-4, | C24H32N2O9 | 492.53 |
| | MALEATE | | | 75847-73-3 | | |
| | | | | [enalapril] | | |
| 01500277 | ERGONOVINE | 402002 | H06 | 129-51-1, 60-79-7 | C23H27N3O6 | 441.49 |
| | MALEATE | | | [ergonovine] | | |
| 01501176 | ERYTHROMYCIN | 402012 | G05 | 134-36-1, 114-07-8 | C52H97NO18S | 1056.41 |
| | ESTOLATE | | | [erythromycin] | | |
| 01500288 | ETHAMBUTOL | 402002 | F07 | 1070-11-7, 74-55-5 | C10H26Cl2N2O2 | 277.24 |
| | HYDROCHLORIDE | | | [ethambutol] | | |
| 01502196 | ETHOSUXIMIDE | 402012 | E11 | 77-67-8 | C7H11NO2 | 141.17 |
| 01501005 | ETODOLAC | 402005 | B09 | 41340-25-4 | C17H21NO3 | 287.36 |
| 01505203 | EZETIMIBE | 402009 | H05 | 163222-33-1 | C24H21F2NO3 | 409.44 |
| 01505201 | FAMCICLOVIR | 402009 | F05 | 104227-87-4 | C14H19N5O4 | 321.34 |
| 01501003 | FAMOTIDINE | 402005 | H08 | 76824-35-6 | C8H15N7O2S3 | 337.45 |
| 01501010 | FENOFIBRATE | 402005 | F09 | 49562-28-9 | C20H21ClO4 | 360.84 |
| 01500993 | FLUNARIZINE | 402011 | B02 | 30484-77-6, | C26H28Cl2F2N2 | 477.43 |
| | HYDROCHLORIDE | | | 52468-60-7 | | |
| | | | | [flunarazine] | | |
| 01504173 | FLUOXETINE | 402012 | H03 | 54910-89-3 | C17H19ClF3NO | 345.80 |
| 01500994 | FLUPHENAZINE | 402005 | G08 | 146-56-5 | C22H28Cl2F3N3OS | 510.45 |
| | HYDROCHLORIDE | | | | | |
| 01500308 | FLURBIPROFEN | 402002 | F08 | 5104-49-4 | C15H13FO2 | 244.27 |
| 01502039 | FOSFOMYCIN | 402006 | D08 | 26472-47-9, | C3H5CaO4P | 176.12 |
| | | | | 23112-90-5(acid) | | |
| 01500310 | FUROSEMIDE | 402002 | H08 | 54-31-9 | C12H11ClN2O5S | 330.75 |
| 01500313 | GEMFIBROZIL | 402002 | C09 | 25812-30-0 | C15H22O3 | 250.34 |
| 01504145 | GLICLAZIDE | 402008 | A10 | 21187-98-4 | C15H21N3O3S | 323.42 |
| 02300229 | GLYBURIDE | 402010 | A09 | 10238-21-8 | C23H28ClN3O5S | 494.01 |
| 01500321 | GUAIFENESIN | 402002 | G09 | 93-14-1 | C10H14O4 | 198.22 |
| 01500325 | HALOPERIDOL | 402002 | C10 | 52-86-8 | C21H23ClFNO2 | 375.87 |
| 01500330 | HEXYLRESORCINOL | 402002 | F10 | 136-77-6 | C12H18O2 | 194.28 |
| 01500334 | HYDRALAZINE | 402002 | B11 | 304-20-1, 86-54-4 | C8H9ClN4 | 196.64 |
| | HYDROCHLORIDE | | | [hydralazine] | | |
| 01500335 | HYDROCHLOROTH | 402002 | C11 | 58-93-5 | C7H8ClN3O452 | 297.74 |
| | IAZIDE | | | | | |
| 01503978 | HYDROXYCHLOR | 402012 | C11 | 747-36-4, 118-42-3 | C18H28ClN3O5S | 433.96 |
| | OQUINE SULFATE | | | [hydroxychloroquine] | | |
| 01500344 | HYDROXYUREA | 402002 | G11 | 127-07-1 | CH4N2O2 | 76.06 |
| 01500345 | HYDROXYZINE | 402002 | H11 | 10246-75-0, | C44H43ClN2O8 | 763.29 |
| | PAMOATE | | | 68-88-2 | | |
| | | | | [hydroxyzine] | | |
| 01500347 | IBUPROFEN | 402003 | C02 | 15687-27-1, | Cl3H18O2 | 206.29 |
| | | | | 58560-75-1 | | |
| | | | | [(+/-) mixture] | | |
| 01500348 | IMIPRAMINE | 402003 | D02 | 113-52-0, 50-49-7 | Cl9H25ClN2 | 316.88 |
| | HYDROCHLORIDE | | | [imipramine] | | |
| 01500349 | INDAPAMIDE | 402003 | E02 | 26807-65-8 | Cl6H16ClN3O3S | 365.84 |
| 01500350 | INDOMETHACIN | 402003 | F02 | 53-86-1 | Cl9H16ClNO4 | 357.80 |
| 01500354 | IPRATROPIUM | 402013 | F04 | 66985-17-9, | C20H30BrNO3 | 412.37 |
| | BROMIDE | | | 22254-24-6 | | |
| | | | | [anhydrous] | | |
| 01504259 | IRBESARTAN | 402009 | H03 | 138402-11-6 | C25H28N6O | 428.54 |
| 01500355 | ISONIAZID | 402003 | A03 | 54-85-3 | C6H7N3O | 137.14 |
| 01500358 | ISOSORBIDE | 402003 | D03 | 87-33-2 | C6H8N2O8 | 236.14 |
| | DINITRATE | | | | | |
| 01500362 | KETOCONAZOLE | 402003 | G03 | 65277-42-1 | C26H28Cl2N4O4 | 531.44 |
| 01501215 | KETOPROFEN | 402006 | C06 | 22071-15-4 | Cl6H14O3 | 254.29 |
| 01503925 | KETOROLAC | 402012 | D10 | 74103-07-4, | Cl9H24N2O6 | 376.41 |
| | TROMETHAMINE | | | 74103-06-3 | | |
| | | | | [ketorolac] | | |
| 01500668 | KETOTIFEN | 402005 | A02 | 34580-14-8, | C23H23NO5S | 425.51 |
| | FUMARATE | | | 34580-13-7 | | |
| | | | | [ketotifen] | | |
| 01503243 | LABETALOL | 402007 | C10 | 32780-64-6, | C19H25ClN2O3 | 364.88 |
| | HYDROCHLORIDE | | | 36894-69-6 | | |
| | | | | [labetalol] | | |
| 01500363 | LACTULOSE | 402013 | F10 | 4618-18-2 | C12H22O11 | 342.30 |
| 01503926 | LANSOPRAZOLE | 402008 | F06 | 103577-45-3 | C16H14F3N3O2S | 369.37 |
| 01500364 | LEUCOVORIN | 402003 | H03 | 1492-18-8 | C20H21CaN7O7 | 511.51 |
| | CALCIUM | | | | | |
| 02300205 | LEVODOPA | 402010 | H08 | 59-92-7 | C9H11NO4 | 197.19 |
| 01504260 | LEVOFLOXACIN | 402009 | A04 | 138199-71-0 | C18H20FN3O4 | 361.38 |
| 01502047 | LIOTHYRONINE | 402006 | B09 | 55-06-1, 6893-02-3 | C15H11I3NNaO4 | 672.96 |
| | SODIUM | | | [liothyronine] | | |
| 01501217 | LISINOPRIL | 402006 | D06 | 83915-83-7, | C21H31N3O5 | 405.50 |
| | | | | 76547-98-3 | | |
| | | | | [anhydrous] | | |
| 02300241 | LOPERAMIDE | 402013 | A06 | 34552-83-5, | C29H34Cl2N2O2 | 513.51 |
| | HYDROCHLORIDE | | | 53179-11-6 | | |
| | | | | [loperamide] | | |
| 01503712 | LORATADINE | 402008 | F05 | 79794-75-5 | C22H23ClN2O2 | 382.89 |
| 01504268 | LOSARTAN | 402009 | D04 | 124750-99-8, | C22H23ClN6O | 422.92 |
| | | | | 114798-26-4 | | |
| | | | | [losartan] | | |
| 01503977 | LOVASTATIN | 402008 | D07 | 75330-75-5 | C24H36O5 | 404.55 |
| 02300242 | LOXAPINE | 402012 | H10 | 27833-64-3, | C22H24ClN3O5 | 445.91 |
| | SUCCINATE | | | 1977-10-2 | | |
| | | | | [loxapine] | | |
| 01500373 | MAPROTILINE | 402003 | D04 | 10347-81-6, | C20H24ClN | 313.87 |
| | HYDROCHLORIDE | | | 10262-69-8 | | |
| | | | | (maprotiline) | | |
| 01501110 | MEBENDAZOLE | 402005 | H10 | 31431-39-7 | C16H13N3O3 | 295.30 |
| 01501103 | MEFENAMIC ACID | 402013 | B02 | 61-68-7 | C15H15NO2 | 241.29 |
| 01503070 | MEFLOQUINE | 402007 | E07 | 53230-10-7 | C17H16F6N2O | 378.32 |
| 01504150 | MELOXICAM | 402008 | C10 | 71125-38-7 | C14H13N3O4S2 | 351.41 |
| 01501121 | MEMANTINE | 402005 | H11 | 19982-08-2 | C12H22ClN | 215.77 |
| | HYDROCHLORIDE | | | | | |
| 01500387 | MERCAPTOPURINE | 402003 | E05 | 6112-76-1, 50-44-2 | C5H4N4S | 152.18 |
| | | | | [anhydrous] | | |
| 01503252 | METHAZOLAMIDE | 402011 | G10 | 554-57-4 | C5H8N4O3S2 | 236.27 |
| 01500394 | METHENAMINE | 402003 | G05 | 100-97-0 | C6H12N4 | 140.19 |
| 01500397 | METHOCARBAMOL | 402003 | A06 | 532-03-6 | C11H15NO5 | 241.25 |
| 01500398 | METHOTREXATE | 402003 | B06 | 59-05-2 | C20H22N8O5 | 454.45 |
| 01500400 | METHOXSALEN | 402003 | C06 | 298-81-7 | C12H8O4 | 216.20 |
| 01500403 | METHYLDOPA | 402003 | E06 | 41372-08-1, | C10H13NO4 | 211.22 |
| | | | | 555-30-6 | | |
| | | | | [anhydrous] | | |
| 01500410 | METOCLOPRAMIDE | 402003 | F06 | 54143-57-6, | C14H23Cl2N3O2 | 336.26 |
| | HYDROCHLORIDE | | | 7232-21-5 | | |
| | | | | [anhydrous], | | |
| | | | | 364-62-5 | | |
| | | | | [metoclopramide] | | |
| 02300325 | METOLAZONE | 402012 | F11 | 17560-51-9 | C16H16ClN3O3S | 365.84 |
| 01500411 | METOPROLOL | 402003 | G06 | 56392-17-7, | Cl9H31NO9 | 417.46 |
| | TARTRATE | | | 37350-58-6 | | |
| | | | | [metroprolol] | | |
| 01500412 | METRONIDAZOLE | 402003 | H06 | 443-48-1, | C6H9N3O3 | 171.16 |
| | | | | 69198-10-3 | | |
| | | | | [metronidazole | | |
| | | | | hydrochloride] | | |
| 01503257 | MIDODRINE | 402012 | A08 | 3092-17-9, | C12H19ClN2O4 | 290.75 |
| | HYDROCHLORIDE | | | 42794-76-3 | | |
| | | | | [midodrine] | | |
| 01500415 | MINOXIDIL | 402003 | B07 | 38304-91-5 | C9H15N5O | 209.25 |
| 01503278 | MITOXANTHRONE | 402007 | F11 | 70476-82-3, | C22H30Cl2N4O6 | 517.41 |
| | HYDROCHLORIDE | | | 65271-80-9 | | |
| | | | | [mitoxantrone] | | |
| 01505361 | MODAFINIL | 402010 | F05 | 68693-11-8 | C15H15NO2S | 273.36 |
| 01504303 | MOXIFLOXACIN | 402009 | A05 | 186826-86-8 | C23H29ClFN3O4 | 465.96 |
| | HYDROCHLORIDE | | | | | |
| 01500674 | MYCOPHENOLIC | 402005 | A03 | 24280-93-1 | C17H20O6 | 320.35 |
| | ACID | | | | | |
| 01503650 | NABUMETONE | 402012 | A09 | 42924-53-8 | C15H16O2 | 228.29 |
| 01503260 | NADOLOL | 402012 | B07 | 42200-33-9 | C17H27NO4 | 309.41 |
| 01500422 | NALOXONE | 402003 | E07 | 357-08-4, | C19H22ClNO4 | 363.84 |
| | HYDROCHLORIDE | | | 51481-60-8 | | |
| | | | | [dihydrate], | | |
| | | | | 465-65-6 | | |
| | | | | [naloxone] | | |
| 01503262 | NALTREXONE | 402012 | C07 | 16676-29-2, | C20H23NO4 | 341.41 |
| | HYDROCHLORIDE | | | 16590-41-3 | | |
| | | | | [naltrexone] | | |
| 01500425 | NAPROXEN(+) | 402003 | G07 | 22204-53-1 | C14H14O3 | 230.27 |
| 01500428 | NEOSTIGMINE | 402003 | A08 | 114-80-7, 59-99-4 | C12H19BrN2O2 | 303.20 |
| | BROMIDE | | | [neostigmine] | | |
| 01500431 | NIFEDIPINE | 402003 | C08 | 21829-25-4 | C17H18N2O6 | 346.34 |
| 01504152 | NILUTAMIDE | 402012 | D02 | 63612-50-0 | C12H10F3N3O4 | 317.23 |
| 01503600 | NIMODIPINE | 402008 | A03 | 66085-59-4 | C21H26N2O7 | 418.45 |
| 01500433 | NITROFURANTOIN | 402003 | D08 | 67-20-9, 54-87-5 | C8H6N4O5 | 238.16 |
| | | | | [nitrofurantoin | | |
| | | | | sodium], | | |
| | | | | 17140-81-7 | | |
| | | | | [monohydrate] | | |
| 01500440 | NORFLOXACIN | 402003 | B09 | 70458-96-7 | C16H18FN3O3 | 319.34 |
| 01500442 | NORTRIPTYLINE | 402003 | D09 | 894-71-3, 72-69-5 | C19H21N | 263.39 |
| | | | | [nortriptyline] | | |
| 01500445 | NYLIDRIN | 402003 | G09 | 1400-61-9 | C19H26ClNO2 | 335.88 |
| | HYDROCHLORIDE | | | | | |
| 01505205 | OLMESARTAN | 402009 | B06 | 144689-63-4 | C29H30N6O6 | 558.60 |
| | MEDOXOMIL | | | | | |
| 01504300 | ORLISTAT | 402009 | G04 | 96829-58-2 | C29H53NO5 | 495.75 |
| 01500447 | ORPHENADRINE | 402003 | A10 | 4682-36-4, 83-98-7 | C24H31NO8 | 461.52 |
| | CITRATE | | | [orphenadrine] | | |
| 01504243 | OXCARBAZEPINE | 402009 | D03 | 28721-07-5 | C15H12N2O2 | 252.28 |
| 01503228 | PAROMOMYCIN | 402007 | B11 | 1263-89-4, | C23H47N5O18S | 713.72 |
| | SULFATE | | | 7542-37-2 | | |
| | | | | [paromomycin | | |
| | | | | 1,59-04-1 | | |
| | | | | [paromomycin,, | | |
| | | | | replaced] | | |
| 01503611 | PENTOXIFYLLINE | 402012 | E08 | 6493-05-6 | C13H18N4O3 | 278.31 |
| 01503936 | PERICIAZ1NE | 402008 | B07 | 2622-26-6 | C21H23N3OS | 365.50 |
| 01505212 | PERINDOPRIL | 402009 | H06 | 107133-36-8; | C23H43N3O5 | 441.62 |
| | ERBUM1NE | | | 82834-16-0 | | |
| | | | | (perindopril) | | |
| 01503934 | PERPHENAZINE | 402011 | H03 | 58-39-9 | C21H26ClN3OS | 403.98 |
| 01500473 | PHENAZOPYRIDINE | 402003 | C11 | 136-40-3, 94-78-0 | C11H12ClN5 | 249.70 |
| | HYDROCHLORIDE | | | [phenazopyridine] | | |
| 01500476 | PHENELZINE | 402003 | D11 | 156-51-4, 51-71-8 | C8H14N2O4S | 234.28 |
| | SULFATE | | | [phenelzine] | | |
| 01500485 | PHENYTOIN | 402003 | G11 | 630-93-3, 57-41-0 | C15H11N2NaO2 | 274.26 |
| | SODIUM | | | [phenytoin] | | |
| 01501134 | PIMOZIDE | 402006 | H02 | 2062-78-4 | C28H29F2N3O | 461.56 |
| 01500488 | PINDOLOL | 402013 | C08 | 13523-86-9 | C14H20N2O2 | 248.33 |
| 01504401 | PIOGLITAZONE | 402009 | B05 | 111025-46-8 | C19H21ClN2O3S | 392.91 |
| | HYDROCHLORIDE | | | (pioglitazone) | | |
| 01500491 | PIROXICAM | 402013 | D09 | 36322-90-4 | C15H13N3O4S | 331.35 |
| 01500113 | POTASSIUM p- | 402001 | C03 | 150-13-0 | C7H6KNO2 | 175.23 |
| | AMINOBENZOATE | | | (acid) | | |
| 01505803 | PRAVASTATIN | 402010 | A06 | 81131-70-6 | C23H35NaO7 | 446.52 |
| | SODIUM | | | | | |
| 01505816 | PREGABALIN | 402010 | D06 | 148553-50-8 | C8H17NO2 | 159.23 |
| 01500500 | PRIMAQUINE | 402004 | D02 | 63-45-6, 90-34-6 | C15H27N3O9P2 | 455.34 |
| | DIPHOSPHATE | | | [primaquine] | | |
| 01500501 | PRIMIDONE | 402013 | C04 | 125-33-7 | C12H14N2O2 | 218.26 |
| 01500502 | PROBENECID | 402013 | C09 | 57-66-9 | C13H19NO4S | 285.36 |
| 01500503 | PROCAINAMIDE | 402013 | D05 | 614-39-1, 51-06-9 | C13H22ClN3O | 271.79 |
| | HYDROCHLORIDE | | | [procainamide] | | |
| 01500505 | PROCHLORPERAZINE | 402004 | E02 | 1257-78-9, 84-02-6 | C22H30ClN3O6S3 | 564.15 |
| | EDISYLATE | | | [prochlorperazine | | |
| | | | | maleate], | | |
| | | | | 58-38-8 | | |
| | | | | [prochlorperazine] | | |
| 01500507 | PROCYCLIDINE | 402013 | D10 | 1508-76-5, 77-37-2 | C19H30ClNO | 323.91 |
| | HYDROCHLORIDE | | | [procyclidine] | | |
| 01500510 | PROMETHAZINE | 402004 | G02 | 58-33-3, 60-87-7 | C17H21ClN2S | 320.89 |
| | HYDROCHLORIDE | | | [promethazine] | | |
| 01503935 | PROPAFENONE | 402008 | A07 | 34183-22-7, | C21H28ClNO3 | 377.92 |
| | HYDROCHLORIDE | | | 54063-53-5 | | |
| | | | | [propafenone] | | |
| 01505270 | PROPRANOLOL | 402013 | B07 | 318-98-9, 525-66-6 | C16H22ClNO2 | 295.81 |
| | HYDROCHLORIDE (+/−) | | | [propranolol] | | |
| 01500515 | PROPYLTHIOURACIL | 402011 | B07 | 51-52-5 | C7H10N2OS | 170.23 |
| 01500516 | PSEUDOEPHEDRINE | 402004 | B03 | 345-78-8, 90-82-4 | C10H16ClNO | 201.70 |
| | HYDROCHLORIDE | | | [pseudoephedrine] | | |
| 01500517 | PYRANTEL | 402004 | C03 | 22204-24-6, | C34H30N2O6S | 594.69 |
| | PAMOATE | | | 15686-83-6 | | |
| | | | | [pyrantel] | | |
| 01500518 | PYRAZINAMIDE | 402011 | C05 | 98-96-4 | C5H5N3O | 123.12 |
| 01503240 | PYRIDOSTIGMINE | 402007 | A10 | 101-26-8, 155-97-5 | C9H13BrN2O2 | 261.12 |
| | BROMIDE | | | [pyridostigmine] | | |
| 01503076 | QUINAPRIL | 402007 | H07 | 82586-55-8, | C25H31ClN2O5 | 474.99 |
| | HYDROCHLORIDE | | | 85441-61-8 | | |
| | | | | [quinapril] | | |
| 01500524 | QUININE SULFATE | 402004 | G03 | 6119-70-6, | C20H26N2O6S | 422.50 |
| | | | | 804-63-7 | | |
| | | | | [anhydrous], | | |
| | | | | 130-95-0 | | |
| | | | | [quinine] | | |
| 01501151 | RANITIDINE | 402006 | F03 | 66357-35-5 | C13H22N4O35 | 314.41 |
| 01500529 | RIFAMPIN | 402004 | A04 | 13292-46-1 | C43H58N4O12 | 822.96 |
| 01505321 | RIFAXIMIN | 402010 | B03 | 80621-81-4 | C43H51N3O11 | 785.90 |
| 01505348 | RILUZOLE | 402010 | D05 | 1744-22-5 | C8H5F3N2OS | 234.20 |
| 01504263 | ROSIGLITAZONE | 402009 | C04 | 122320-73-4 | C18H19N3O3S | 357.43 |
| 01505213 | ROSUVASTATIN | 402009 | A07 | 287714-14-4, | C22H28FN3O6S | 481.55 |
| | | | | 147098-20- | | |
| | | | | 2(Ca salt) | | |
| 01505262 | SERTRALINE | 402009 | D09 | 79559-97-0; | C17H18Cl3N | 342.70 |
| | HYDROCHLORIDE | | | 79617-96- | | |
| | | | | 2(base) | | |
| 01504099 | SILDENAFIL | 402008 | D09 | 139755-83-2 | C22H30N6O4S | 474.59 |
| 01503423 | SPIRAMYCIN | 402008 | G02 | 8025-81-8 | C43H74N2O14 | 843.07 |
| 01500539 | SPIRONOLACTONE | 402004 | G04 | 52-01-7 | C24H32O4S | 416.58 |
| 01500550 | SULFAMETHOXAZOLE | 402004 | F05 | 723-46-6 | C10H11N3O3S | 253.28 |
| 01500552 | SULFASALAZINE | 402004 | H05 | 599-79-1 | C18H14N4O5S | 398.40 |
| 01500554 | SULFINPYRAZONE | 402011 | A10 | 57-96-5 | C23H20N2O3S | 404.49 |
| 01500555 | SULFISOXAZOLE | 402011 | B08 | 127-69-5 | C11H13N3O3S | 267.31 |
| 01500556 | SULINDAC | 402004 | B06 | 38194-50-2 | C20H17FO3S | 356.42 |
| 01503142 | TENOXICAM | 402007 | D09 | 59804-37-4 | C13H11N3OS2 | 337.38 |
| 01500566 | TETRACYCLINE | 402004 | C06 | 64-75-5, 60-54-8 | C22H25ClN2O8 | 480.91 |
| | HYDROCHLORIDE | | | [tetracycline] | | |
| 01500568 | THEOPHYLLINE | 402004 | D06 | 5967-84-0, 58-55-9 | C7H8N4O2 | 180.17 |
| | | | | [anhydrous] | | |
| 01500573 | THIOGUANINE | 402004 | G06 | 154-42-7, | C5H5N5S | 167.19 |
| | | | | 5580-03-0 | | |
| | | | | [hemihydrate] | | |
| 01500576 | THIOTHIXENE | 402011 | C04 | 5591-45-7, | C23H29N3O2S2 | 443.63 |
| | | | | 3313-26-6 | | |
| | | | | [//Z//] | | |
| 01500578 | TIMOLOL | 402004 | H06 | 26921-17-5, | C17H28N4O7S | 432.50 |
| | MALEATE | | | 91524-16-2 | | |
| | | | | [timolol] | | |
| 01500581 | TOLBUTAMIDE | 402004 | A07 | 64-77-7 | C12H18N2O3S | 270.35 |
| 01501198 | TOLFENAMIC ACID | 402006 | F05 | 13710-19-5 | C14H12ClNO2 | 261.71 |
| 01505801 | TOPIRAMATE | 402010 | G05 | 97240-79-4 | C12H21NO8S | 339.37 |
| 01505264 | TRANDOLAPRIL | 402009 | F09 | 87679-37-6 | C24H34N2O5 | 430.55 |
| 01502026 | TRANEXAMIC ACID | 402006 | G07 | 1197-18-8 | C8H15NO2 | 157.21 |
| 01500584 | TRANYLCYPROMINE | 402004 | C07 | 13492-01-8, | C9H13NO4S | 231.27 |
| | SULFATE | | | 7081-36-9 | | |
| | | | | [replaced], | | |
| | | | | 155-09-9 | | |
| | | | | [tranylcypromine] | | |
| 01503121 | TRAZODONE | 402007 | H08 | 25332-39-2, | C19H23Cl2N5O | 408.33 |
| | HYDROCHLORIDE | | | 19794-93-5 | | |
| | | | | [trazodone] | | |
| 01500591 | TRIFLUOPERAZINE | 402004 | A08 | 440-17-5, 117-89-5 | C21H26Cl2F3N3S | 480.43 |
| | HYDROCHLORIDE | | | [trifluoperazine] | | |
| 01500592 | TRIHEXYPHENIDYL | 402004 | B08 | 52-49-3 | C20H32ClNO | 337.94 |
| | HYDROCHLORIDE | | | | | |
| 01500593 | TRIMEPRAZINE | 402004 | C08 | 4330-99-8, | C22H28N2O6S | 448.54 |
| | TARTRATE | | | 41375-66-0 | | |
| | | | | [replaced], 84-96-8 | | |
| | | | | [trimeprazine] | | |
| 01500595 | TRIMETHOPRIM | 402004 | E08 | 738-70-5 | C14H18N4O3 | 290.32 |
| 01503117 | TRIMIPRAMINE | 402012 | E04 | 521-78-8, 739-71-9 | C24H30N2O4 | 410.52 |
| | MALEATE | | | [trimipramine] | | |
| 01500605 | URSODIOL | 402004 | D09 | 128-13-2 | C24H40O4 | 392.58 |
| 01505209 | VALSARTAN | 402009 | E06 | 137862-53-4 | C24H28N5NaO3 | 457.51 |
| | SODIUM | | | (valsartan) | | |
| 01500607 | VANCOMYCIN | 402004 | E09 | 1404-93-9, | C67H77Cl3N8O24 | 1484.76 |
| | HYDROCHLORIDE | | | 1404-90-6 | | |
| | | | | [vancomycin] | | |
| 01504171 | VENLAFAXINE | 402008 | F10 | 99300-78-4, | C17H27NO2 | 277.41 |
| | | | | 93413-69-5 | | |
| | | | | [venlafaxine] | | |
| 02300307 | VERAPAMIL | 402013 | B03 | 152-11-4, 52-53-9 | C27H39ClN2O4 | 491.08 |
| | HYDROCHLORIDE | | | [verapamil] | | |
| 01500663 | YOHIMBINE | 402005 | B02 | 65-19-0 | C21H27ClN2O3 | 390.91 |
| | HYDROCHLORIDE | | | | | |
| 01502109 | ZIDOVUDINE [AZT] | 402012 | B03 | 30516-87-1 | C10H13N5O4 | 267.25 |
| 01505281 | ZOLMITRIPTAN | 402009 | C10 | 139264-17-8 | C16H21N3O2 | 287.36 |
| | | ID | BIOACTIVITY | SOURCE | STATUS | REFERENCES |
| | | 01502057 | NMDA receptor | synthetic | experimental | |
| | | | antagonist (gly) | | | |
| | | 01500665 | antihypertensive, | synthetic | USAN, INN, | |
| | | | antianginal, | | BAN | |
| | | | antiarrhythmic | | | |
| | | 01500101 | analgesic, | synthetic | USP, INN, | |
| | | | antipyretic | | BAN | |
| | | 01500102 | carbonic | synthetic | USP, INN, | |
| | | | anhydrase | | BAN, JAN | |
| | | | inhibitor, diuretic, | | | |
| | | | antiglaucoma | | | |
| | | 01500105 | mucolytic | synthetic | USP, INN, | |
| | | | | | BAN, JAN | |
| | | 01503603 | antiviral | synthetic | USP, INN, | |
| | | | | | BAN, JAN | |
| | | 01500108 | antihyperuricemia, | synthetic | USP, INN, | |
| | | | antigout, | | BAN, JAN | |
| | | | antiurolithic | | | |
| | | 01505204 | 5HT 1B/2D | synthetic | USAN, INN, | |
| | | | receptor agonist | | BAN | |
| | | 01503065 | antineoplastic | synthetic | USP, INN, | |
| | | | | | BAN | |
| | | 01500110 | antiviral, | synthetic | USP, INN, | |
| | | | antiparkinsonian; | | BAN | |
| | | | treatment of drug- | | | |
| | | | induced | | | |
| | | | extrapyrimidal | | | |
| | | | reactions | | | |
| | | 01500111 | antibacterial | semisynthetic | USP, JAN | |
| | | 01500112 | Na+ channel | synthetic | USP, INN, | Biochim Biophys |
| | | | inhibitor, diuretic | | BAN | Acta 944: 383 |
| | | | | | | (1988) |
| | | 02300165 | adrenergic agonist, | synthetic | USAN, INN, | Adv Drug Res |
| | | | coronary | | BAN, JAN | 16: 309 (1987) |
| | | | vasodilator, Ca | | | |
| | | | channel blocker | | | |
| | | 01500117 | antidepressant | synthetic | USP, INN, | |
| | | | | | BAN, JAN | |
| | | 01505202 | Ca channel | synthetic | USAN, INN, | |
| | | | blocker | | BAN, JAN | |
| | | 01500120 | antibacterial | semisynthetic | USP, INN, | |
| | | | | | BAN, JAN | |
| | | 01500122 | antifungal | Streptomycetes | USP, INN, | New Engl J Med |
| | | | | nodosus | BAN, JAN | 296: 784 (1977) |
| | | 01500128 | analgesic | synthetic | USP, INN, | |
| | | | | | BAN, JAN | |
| | | 01500130 | analgesic, | synthetic | USP, BAN, | |
| | | | antipyretic, | | JAN | |
| | | | antiinflammatory | | | |
| | | 01501127 | beta adrenergic | synthetic | USP, INN, | |
| | | | blocker | | BAN, JAN | |
| | | 01503722 | antihyperlipidemic, | synthetic | USAN, INN, | |
| | | | HMGCoA | | BAN | |
| | | | reductase inhibitor | | | |
| | | 01504210 | antipneumocystic, | synthetic | USP, INN, | |
| | | | antimalarial | | BAN | |
| | | 01500133 | immunosuppressant, | synthetic | USP, INN, | |
| | | | antineoplastic, | | BAN, JAN | |
| | | | antirheumatic | | | |
| | | 01503679 | antibacterial | semisynthetic | USP, INN, BAN | |
| | | 01500134 | antibacterial | Bacillus | USP, INN, | |
| | | | | licheniformis | BAN, JAN | |
| | | | | and B subtilis | | |
| | | 01500135 | muscle relaxant | synthetic | USP, INN, | |
| | | | (skeletal) | | BAN, JAN | |
| | | 01505200 | ACE inhibitor, | synthetic | USAN, INN, | |
| | | | antihypertensive | | BAN, JAN | |
| | | 01500137 | decarboxylase | component of | USAN, INN, | |
| | | | inhibitor | Madopa | BAN, JAN | |
| | | | | (Hoffmann- | | |
| | | | | LaRoche) | | |
| | | 01500142 | anticholinergic | synthetic | USP, INN, | |
| | | | | | BAN, JAN | |
| | | 01500146 | cholinergic | synthetic | USP, BAN, JAN | |
| | | 01502046 | antihyperlipidemic | synthetic | USAN, INN, | |
| | | | | | BAN, JAN | |
| | | 01500147 | cathartic | synthetic | USP, INN, | |
| | | | | | BAN, JAN | |
| | | 01503985 | H1 antihistamine | synthetic | USP, INN, BAN | |
| | | 01500813 | antiinflammatory | semisynthetic | USAN, INN, | |
| | | | | | BAN, JAN | |
| | | 01502004 | diuretic | synthetic | USP, INN, | |
| | | | | | BAN, JAN | |
| | | 01504174 | antidepressant | synthetic | USP, INN, BAN | |
| | | 01500152 | antineoplastic, | synthetic | USP, INN, | |
| | | | alkylating agent | | BAN, JAN | |
| | | 01504261 | angiotensin 1 | synthetic | USAN, INN | |
| | | | receptor antagonist | | | |
| | | 01500682 | antihypertensive | synthetic | USP, INN, | |
| | | | | | BAN, JAN | |
| | | 01500158 | cholinergic, miotic | synthetic | USP, INN, | |
| | | | | | BAN, JAN | |
| | | 01500159 | analgesic, | synthetic | USP, INN, | |
| | | | anticonvulsant | | BAN, JAN | |
| | | 01504257 | betaadrenergic | synthetic | USAN, INN, | |
| | | | blocker | | BAN, JAN | |
| | | 01500771 | antibacterial | semisynthetic | USP, INN, | |
| | | | | | BAN, JAN | |
| | | 01500163 | antibacterial | semisynthetic | USP, INN, | |
| | | | | | BAN, JAN | |
| | | 01502028 | antibacterial | semisynthetic | USP, INN, | |
| | | | | | BAN, JAN | |
| | | 01500183 | antihistaminic | synthetic | USP, INN, BAN | |
| | | 01500184 | antiemetic, | synthetic | USP, INN, | |
| | | | antipsychotic | | BAN, JAN | |
| | | 01500185 | antidiabetic | synthetic | USP, INN, | |
| | | | | | BAN, JAN | |
| | | 01500187 | diuretic, | synthetic | USP, INN, | |
| | | | antihypertensive | | BAN, JAN | |
| | | 01500684 | antiulcerative | synthetic | USP, INN, | |
| | | | | | BAN, JAN | |
| | | 01503614 | antibacterial, | synthetic | USP, INN, | |
| | | | fungicide | | BAN | |
| | | 01504231 | antibacterial | Streptomyces | USP, INN, | |
| | | | | erythreus | BAN, JAN | |
| | | 01500191 | antihistaminic | synthetic | USAN, BAN | |
| | | 01500193 | antibacterial, | semisynthetic; | USAN, INN, | |
| | | | inhibits protein | U-21251 | BAN | |
| | | | synthesis | | | |
| | | 02300061 | antidepressant | synthetic | USP, INN, | |
| | | | | | BAN, JAN | |
| | | 01500198 | antihypertensive | synthetic | USP, INN, | |
| | | | | | BAN | |
| | | 01503710 | platelet | synthetic | USP, INN, | |
| | | | aggregation | | BAN | |
| | | | inhibitor | | | |
| | | 01500200 | antifungal | synthetic | USP, INN, | |
| | | | | | BAN, JAN | |
| | | 01500201 | antibacterial | semisynthetic | USP, INN, | |
| | | | | | BAN, JAN | |
| | | 01500685 | antipsychotic | synthetic | USP, INN, BAN | |
| | | 01500205 | antimitotic, | Colchicum | USP, JAN | J Am Chem Soc |
| | | | antigout agent | autumnale | | 74: 487 (1952) |
| | | 01500209 | antiinfectant | coal tar | NF, JAN | |
| | | 01500210 | antiasthmatic, | synthetic | USP, INN, | |
| | | | antiallergy | | BAN, JAN | |
| | | 01503207 | muscle relaxant | synthetic | USP, INN | |
| | | | (skeletal) | | | |
| | | 01500213 | antineoplastic, | synthetic | USP, INN, | |
| | | | alkylating agent | | BAN, JAN | |
| | | 01502202 | immunosuppressant | Tolypocladium | USP, INN, | Helv Chim Acta |
| | | | | inflatum | BAN, JAN | 60: 1568 (1977) |
| | | 01500220 | anterior pituitary | synthetic | USP, INN, | |
| | | | suppressant | | BAN, JAN | |
| | | 01500222 | antibacterial, | synthetic | USP, INN, | |
| | | | leprostatic, | | BAN | |
| | | | dermatitis | | | |
| | | | herpetiformis | | | |
| | | | suppressant | | | |
| | | 01503127 | antiinfectant | synthetic; BAQD-10 | INN, BAN, JAN | |
| | | 01500227 | antidepressant | synthetic | USP, INN, | |
| | | | | | BAN, JAN | |
| | | 01500233 | antitussive | synthetic | USP, INN, BAN | |
| | | 02300206 | antihypertensive, | synthetic; | USP, INN, | |
| | | | diuretic, activates | SCH-6783; | BAN | |
| | | | K channels and | NSC-64198 | | |
| | | | AMPA receptors | | | |
| | | 01500237 | antiinflammatory | synthetic | USP, JAN | |
| | | 01500245 | analgesic, | synthetic | USP, INN, | |
| | | | antiinflammatory | | BAN, JAN | |
| | | 01500247 | cardiac stimulant | Digitalis | USP, INN, | J. Chem.Soc.1930: |
| | | | | lanata or D. orientalis | BAN, JAN | 508; 1954: 2012 |
| | | | | Lam., Scrophulariaceae | | |
| | | 02300214 | Ca channel | synthetic | USP, INN, | |
| | | | blocker, coronary | | BAN, JAN | |
| | | | vasodilator | | | |
| | | 01500251 | antiemetic | synthetic | USP, INN, | |
| | | | | | BAN, JAN | |
| | | 01500256 | antihistaminic | synthetic | USP, INN, | |
| | | | | | BAN, JAN | |
| | | 01500258 | antihistaminic | synthetic | USP-XXI, | |
| | | | | | INN, BAN, JAN | |
| | | 01500259 | coronary | synthetic | USP, INN, | |
| | | | vasodilator | | BAN, JAN | |
| | | 01500261 | antiarrhythmic | synthetic | USP, INN, | |
| | | | | | BAN, JAN | |
| | | 01500264 | antidepressant | synthetic | USP, INN, BAN | |
| | | 01500266 | antibacterial | semisynthetic; GS-3065 | USP, INN, BAN | |
| | | 01500267 | antihistaminic, | synthetic | USP, INN, | |
| | | | hypnotic | | BAN | |
| | | 02300219 | acetylcholinesteras | synthetic | USP, INN, | |
| | | | e inhibitor | | BAN, JAN | |
| | | 01501214 | ACE inhibitor, | synthetic | USP, INN, | |
| | | | antihypertensive | | BAN, JAN | |
| | | 01500277 | oxytocic, 5HT | ergot and | USP, INN, | |
| | | | antagonist | Convolvulva ceae spp | BAN, JAN | |
| | | 01501176 | antibacterial | Streptomyces | USP, INN, | |
| | | | | erythreus | BAN, JAN | |
| | | 01500288 | antibacterial | synthetic | USP, INN, | |
| | | | (tuberculostatic) | | BAN, JAN | |
| | | 01502196 | anticonvulsant | synthetic | USP, INN, | |
| | | | | | BAN, JAN | |
| | | 01501005 | antiinflammatory | synthetic | USP, INN, BAN | |
| | | 01505203 | sterol absorption | synthetic | USAN, INN, | |
| | | | inhibitor | | BAN | |
| | | 01505201 | antiviral | synthetic | USAN, INN, BAN | |
| | | 01501003 | H2 antihistamine | synthetic | USP, INN, | |
| | | | | | BAN, JAN | |
| | | 01501010 | antihyperlipidemic | synthetic | INN, BAN | |
| | | 01500993 | vasodilator | synthetic | USAN, INN, | |
| | | | | | BAN, JAN | |
| | | 01504173 | antidepressant | synthetic | USAN, INN, BAN | |
| | | 01500994 | H1 antihistamine | synthetic | USP, BAN, JAN, | |
| | | 01500308 | antiinflammatory, | synthetic | USP, INN, | |
| | | | analgesic | | BAN, JAN | |
| | | 01502039 | antibacterial | Streptomyces spp | USAN, INN, BAN | |
| | | 01500310 | diuretic, | synthetic | USP, INN, | |
| | | | antihypertensive | | BAN, JAN | |
| | | 01500313 | antihyperlipoprotei | synthetic | USP, INN, | |
| | | | nemic | | BAN | |
| | | 01504145 | antidiabetic | synthetic; | INN, BAN, | Metabolism 50: |
| | | | | SE-1702 | JAN | 688 (2001) |
| | | 02300229 | antihyperglycemic | synthetic | USP, INN, | |
| | | | | | BAN, JAN | |
| | | 01500321 | expectorant | synthetic | USP, INN, | |
| | | | | | BAN, JAN | |
| | | 01500325 | antidyskinetic, | synthetic | USP, INN, | |
| | | | antipsychotic | | BAN, JAN | |
| | | 01500330 | anthelmintic, | synthetic | USP, BAN | |
| | | | topical antiseptic | | | |
| | | 01500334 | antihypertensive | semisynthetic | USP, INN, BAN | |
| | | 01500335 | diuretic | semisynthetic | USP, INN, | |
| | | | | | BAN, JAN | |
| | | 01503978 | antimalarial, lupus | synthetic | USP-XXII, | |
| | | | suppressant | | INN | |
| | | 01500344 | antineoplastic, | synthetic | USP, INN, | |
| | | | inhibits | | BAN | |
| | | | ribonucleoside | | | |
| | | | diphosphate | | | |
| | | | reductase | | | |
| | | 01500345 | anxiolytic, | synthetic | USP, JAN | |
| | | | antihistaminic | | | |
| | | 01500347 | antiinflammatory | synthetic | USP, INN, | |
| | | | | | BAN, JAN | |
| | | 01500348 | antidepressant | synthetic | USP, INN, | |
| | | | | | BAN, JAN | |
| | | 01500349 | diuretic, | synthetic | USP, INN, | |
| | | | antihypertensive | | BAN, JAN | |
| | | 01500350 | antiinflammatory, | synthetic | USP, INN, | |
| | | | antipyretic, | | BAN, JAN | |
| | | | analgesic | | | |
| | | 01500354 | bronchodilator, | synthetic | USAN, INN, | |
| | | | antiarrhythmic | | BAN, JAN | |
| | | 01504259 | angiotensin 2 | synthetic | USP, INN, | |
| | | | receptor antagonist | | BAN | |
| | | 01500355 | antibacterial, | synthetic | USP, INN, | |
| | | | tuberculostatic | | BAN, JAN | |
| | | 01500358 | antianginal | synthetic | USP, INN, | |
| | | | | | BAN, JAN | |
| | | 01500362 | antifungal | synthetic | USP, INN, | |
| | | | | | BAN, JAN | |
| | | 01501215 | antiinflammatory | synthetic | USP, INN, | |
| | | | | | BAN, JAN | |
| | | 01503925 | antiinflammatory | synthetic | USP, INN, BAN | |
| | | 01500668 | antiasthmatic | synthetic | USAN, INN, | |
| | | | | | BAN, JAN | |
| | | 01503243 | adrenergic blocker | synthetic | USP, INN, | |
| | | | | | BAN, JAN | |
| | | 01500363 | laxative | synthetic | USP, INN, | |
| | | | | | BAN, JAN | |
| | | 01503926 | antiulcerative | synthetic | USP, INN, BAN | |
| | | 01500364 | antianemic, | synthetic | USP, INN, | |
| | | | antidote to folic | | BAN, JAN | |
| | | | acid antagonists | | | |
| | | 02300205 | antiparkinsonian | Vicia faba | USP, INN, | |
| | | | | seedlings, | BAN, JAN | |
| | | | | Sarothamnus | | |
| | | | | spp, & other | | |
| | | | | plants | | |
| | | 01504260 | antibacterial | synthetic | USAN, INN, | |
| | | | | | BAN, JAN | |
| | | 01502047 | thyroid hormone | synthetic; L-isomer | USP, BAN, JAN | |
| | | 01501217 | ACE inhibitor | synthetic | USP, INN, | |
| | | | | | BAN, JAN | |
| | | 02300241 | Ca channel | synthetic | USP, INN, | |
| | | | blocker | | BAN, JAN | |
| | | 01503712 | H1 antihistamine | synthetic | USP, INN, BAN | |
| | | 01504268 | antihypertensive, | synthetic | USAN, INN, | |
| | | | AT1 angiotensin II | | BAN | |
| | | | antagonist | | | |
| | | 01503977 | antihyperlipidemic, | synthetic | USP, INN, | PNAS 77:3957 |
| | | | HMGCoA | | BAN | (1980); Int J |
| | | | reductase inhibitor | | | Oncol 12:717 |
| | | | | | | (1998) |
| | | 02300242 | antipsychotic | synthetic | USP | |
| | | 01500373 | antidepressant | synthetic | USAN, INN, BAN | |
| | | 01501110 | anthelmintic | synthetic | USP, INN, | |
| | | | | | BAN, JAN | |
| | | 01501103 | antiinflammatory, | synthetic | USP, INN, | |
| | | | analgesic | | BAN, JAN | |
| | | 01503070 | antimalarial | synthetic | USAN, INN, BAN | |
| | | 01504150 | antiinflammatory | synthetic | USAN, INN, | Neuropharmacol |
| | | | | | BAN | 39: 1653 (2000) |
| | | 01501121 | muscle relaxant | synthetic | USAN | |
| | | | (skeletal) | | | |
| | | 01500387 | antineoplastic, | synthetic | USP, INN, | |
| | | | purine | | BAN, JAN | |
| | | | antimetabolite | | | |
| | | 01503252 | carbonic | synthetic | USP, INN, | |
| | | | anhydrase | | BAN, JAN | |
| | | | inhibitor | | | |
| | | 01500394 | antibacterial | synthetic | USP, INN, | |
| | | | (urinary) | | JAN | |
| | | 01500397 | muscle relaxant | synthetic | USP, INN, | |
| | | | (skeletal) | | BAN, JAN | |
| | | 01500398 | antineoplastic, | synthetic | USP, INN, | |
| | | | antirheumatic, | | BAN, JAN | |
| | | | folic acid | | | |
| | | | antagonist | | | |
| | | 01500400 | antipsoriatic, | synthetic | USP, BAN, | |
| | | | pigmentation agent | | JAN | |
| | | 01500403 | antihypertensive | synthetic | USP, INN, | |
| | | | | | BAN, JAN | |
| | | 01500410 | antiemetic | synthetic | USP, INN, | |
| | | | | | BAN, JAN | |
| | | 02300325 | diuretic, | synthetic | USP, INN, | |
| | | | antihypertensive | | BAN, JAN | |
| | | 01500411 | antihypertensive, | synthetic | USP, JAN | |
| | | | antianginal | | | |
| | | 01500412 | antiprotozoal | synthetic | USP, INN, | |
| | | | | | BAN, JAN | |
| | | 01503257 | antihypertensive, | synthetic | USAN, INN, | |
| | | | vasoconstrictor | | BAN, JAN | |
| | | 01500415 | antihypertensive, | synthetic | USP, INN, | |
| | | | antialopecia agent | | BAN | |
| | | 01503278 | antineoplastic | semisynthetic | USP, INN, | |
| | | | | | BAN, JAN | |
| | | 01505361 | analeptic | synthetic; | USAN, INN, | |
| | | | | CRL-40476, | BAN | |
| | | | | CEP-1538 | | |
| | | 01504303 | antibacterial | synthetic | USAN | |
| | | 01500674 | antineoplastic | Penicillium | USAN, INN, | |
| | | | | brevicompact | BAN | |
| | | | | um and other | | |
| | | | | Penicillium | | |
| | | | | spp | | |
| | | 01503650 | antiinflammatory | synthetic | USP, INN, | |
| | | | | | BAN, JAN | |
| | | 01503260 | betaadrenergic | synthetic | USP, INN, | |
| | | | blocker | | BAN, JAN | |
| | | 01500422 | narcotic antagonist | synthetic | USP, INN, | Brain Res |
| | | | | | BAN, JAN | 839:209 (1999); |
| | | | | | | Brit J Pharmacol |
| | | | | | | 127:605 (1999) |
| | | 01503262 | morphine | synthetic | USP | |
| | | | antagonist | | | |
| | | 01500425 | antiinflammatory, | synthetic | USP, INN, | |
| | | | analgesic, | | BAN, JAN | |
| | | | antipyretic | | | |
| | | 01500428 | cholinergic | synthetic | USP, INN, | |
| | | | | | BAN, JAN | |
| | | 01500431 | antianginal, | synthetic | USP, INN, | |
| | | | antihypertensive | | BAN, JAN | |
| | | 01504152 | antiandrogen | synthetic | USAN, INN, | Pharmacotherapy |
| | | | | | BAN | 31: 65 (1997) |
| | | 01503600 | vasodilator | synthetic | USP, INN, BAN | |
| | | 01500433 | antibacterial | synthetic | USP, INN, | |
| | | | | | BAN, JAN | |
| | | 01500440 | antibacterial | synthetic | USP, INN, | |
| | | | | | BAN, JAN | |
| | | 01500442 | antidepressant | synthetic | USP, INN, | |
| | | | | | BAN, JAN | |
| | | 01500445 | vasodilator | synthetic | USP-XII, | |
| | | | (peripheral) | | INN, BAN | |
| | | 01505205 | Angiotensin II | synthetic | USAN, INN, | |
| | | | inhibitor prodrug, | | BAN | |
| | | | antihypertensive | | | |
| | | 01504300 | reversible lipase | synthetic | USAN, INN, | |
| | | | inhibitor, | | BAN | |
| | | | antiobesity | | | |
| | | 01500447 | muscle relaxant | synthetic | USP, INN, | |
| | | | (skeletal), | | BAN | |
| | | | antihistaminic | | | |
| | | 01504243 | antipsychotic | synthetic | USAN, INN, | |
| | | | | | BAN | |
| | | 01503228 | antibacterial, | Streptomyces | USP, INN, | |
| | | SULFATE | antiamebic | rimosis | BAN | |
| | | | | paramomycinus | | |
| | | 01503611 | vasodilator | synthetic | USP, INN, | |
| | | | | | BAN, JAN | |
| | | 01503936 | antipsychotic | synthetic | BAN, JAN | |
| | | 01505212 | antihypertensive, | synthetic; | USAN | |
| | | | ACE inhibitor | S9490-3, | | |
| | | | | McN-A2833-109 | | |
| | | 01503934 | antipsychotic | synthetic | USP, INN, | |
| | | | | | BAN, JAN | |
| | | 01500473 | analgesic | synthetic | USP, INN, BAN | |
| | | 01500476 | antidepressant | synthetic | USP, INN, | |
| | | | | | BAN | |
| | | 01500485 | anticonvulsant, | synthetic | USP, JAN | |
| | | | antieleptic | | | |
| | | 01501134 | antipsychotic | synthetic | USP, INN, | |
| | | | | | BAN, JAN | |
| | | 01500488 | antihypertensive, | synthetic | USP, INN, | |
| | | | antianginal, | | BAN, JAN | |
| | | | antiarrhythmic, | | | |
| | | | antiglaucoma | | | |
| | | | agent | | | |
| | | 01504401 | antidiabetic | synthetic | USAN, INN, BAN | |
| | | 01500491 | antiinflammatory | synthetic | USP, INN, | |
| | | | | | BAN, JAN | |
| | | 01500113 | ultraviolet screen | synthetic | USP | |
| | | 01505803 | antihyperlipidemic, | CS-514; SQ- | USAN, INN, | |
| | | | HMGCoA | 31000 | BAN, JAN | |
| | | | reductase inhibitor | | | |
| | | 01505816 | anticonvulsant | synthetic; CI-1008 | USAN, INN | |
| | | 01500500 | antimalarial | synthetic | USP, INN, BAN | |
| | | 01500501 | anticonvulsant | synthetic | USP, INN, | |
| | | | | | BAN, JAN | |
| | | 01500502 | uricosuric | synthetic | USP, INN, | |
| | | | | | BAN, JAN | |
| | | 01500503 | antiarrhythmic | synthetic | USP, INN, | |
| | | | | | BAN, JAN | |
| | | 01500505 | antiemetic, | synthetic | USP, JAN | |
| | | | antipsychotic, | | | |
| | | | treatment of | | | |
| | | | vertigo | | | |
| | | 01500507 | anticholinergic | synthetic | USP, INN, BAN | |
| | | 01500510 | antihistaminic | synthetic | USP, INN, | |
| | | | | | BAN, JAN | |
| | | 01503935 | antiarrhythmic | synthetic | USP, INN, | |
| | | | | | BAN, JAN | |
| | | 01505270 | antihypertensive, | synthetic | USP, INN, | |
| | | | antianginal, | | BAN, JAN | |
| | | | antiarrhythmic | | | |
| | | 01500515 | antihyperthyroid | synthetic | USP, INN, | |
| | | | | | BAN, JAN | |
| | | 01500516 | decongestant | synthetic | USP, INN, BAN | |
| | | 01500517 | anthelmintic | synthetic | USP, INN, | |
| | | | | | BAN, JAN | |
| | | 01500518 | antibacterial, | synthetic | USP, INN, | |
| | | | tuberculostatic | | BAN, JAN | |
| | | 01503240 | cholinergic | synthetic | USP, INN, | |
| | | | | | BAN, JAN | |
| | | 01503076 | antihypertensive, | synthetic | USP, INN, | |
| | | | ACE inhibitor | | BAN | |
| | | 01500524 | antimalarial, | Cinchona spp | USP, JAN | |
| | | | skeletal muscle | | | |
| | | | relaxant | | | |
| | | 01501151 | H2 antihistamine | synthetic | USAN, INN, BAN | |
| | | 01500529 | antibacterial | semisynthetic; | USP, INN, | |
| | | | (tuberculostatic) | L-5103, | BAN, JAN | |
| | | | | Ba-41166/E, | | |
| | | | | NSC-113926 | | |
| | | 01505321 | antibacterial, RNA | semisynthetic | USAN, INN | Drugs 49:467 |
| | | | synthesis inhibitor | | | (1995) |
| | | 01505348 | anticonvulsant, | synthetic | USAN, INN, | Neurosci |
| | | | glutamate release | | BAN | Lett140:225 |
| | | | inhibitor | | | (1992); |
| | | | | | | Anesthesiology |
| | | | | | | 76:844 (1992); |
| | | | | | | Fundam Clin |
| | | | | | | Pharmacol 6:177 |
| | | | | | | (1992) |
| | | 01504263 | antidiabetic | synthetic | USAN, INN, BAN | |
| | | 01505213 | antihyperlipidemic | synthetic | USAN, INN, BAN | |
| | | 01505262 | antidepressant, | synthetic | USAN, INN, | |
| | | | 5HT uptake | | BAN | |
| | | | inhibitor | | | |
| | | 01504099 | impotency therapy | synthetic | USAN, INN, BAN | |
| | | 01503423 | antibacterial | Streptomyces | USAN, INN, | J Am Chem Soc |
| | | | | ambofaciens | BAN | 91: 3401 (1969) |
| | | 01500539 | diuretic | synthetic | USP, INN, | |
| | | | | | BAN, JAN | |
| | | 01500550 | antibacterial, | synthetic | USP, INN, | |
| | | | antipneumocystis | | BAN, JAN | |
| | | 01500552 | anticolitis and | synthetic | USP, INN, | |
| | | | Crohn's disease | | BAN | |
| | | 01500554 | uricosuric | synthetic | USP, INN, | |
| | | | | | BAN, JAN | |
| | | 01500555 | antibacterial | synthetic | USP, INN, | |
| | | | | | BAN, JAN | |
| | | 01500556 | antiinflammatory | synthetic | USP, INN, | |
| | | | | | BAN, JAN | |
| | | 01503142 | antiinflammatory | synthetic | USAN, INN, | |
| | | | | | BAN, JAN | |
| | | 01500566 | antibacterial, | Streptomyces | USP, INN, | |
| | | | antiamebic, | spp | BAN, JAN | |
| | | | antirickettsial | | | |
| | | 01500568 | bronchodilator | Camelia, thea, | USP, BAN, JAN | |
| | | | | Paullinia cupana | | |
| | | 01500573 | antineoplastic, | synthetic | USP, INN, | |
| | | | purine | | BAN | |
| | | | antimetabolite | | | |
| | | 01500576 | antipsychotic | synthetic | USP, INN, | |
| | | | | | BAN, JAN | |
| | | 01500578 | betaadrenergic | synthetic | USP, JAN | |
| | | | blocker | | | |
| | | 01500581 | antidiabetic | synthetic | USP, INN, | |
| | | | | | BAN, JAN | |
| | | 01501198 | antiinflammatory, | synthetic | INN, BAN, | |
| | | | analgesia | | JAN | |
| | | 01505801 | anticonvulsant, | synthetic; | USAN, INN, | |
| | | | antimigraine, | RWJ-17021 | BAN | |
| | | | GABA-A agonist, | | | |
| | | | AMP/kinate | | | |
| | | | glutamate receptor | | | |
| | | | antagonist, | | | |
| | | | carbonic | | | |
| | | | anhydrase | | | |
| | | | inhibitor | | | |
| | | 01505264 | antihypertensive, | synthetic | INN, BAN | |
| | | | ACE inhibitor | | | |
| | | 01502026 | hemostatic | synthetic | USAN, INN, | |
| | | | | | BAN, JAN | |
| | | 01500584 | antidepressant | synthetic | USP-XXI, | |
| | | | | | INN, BAN | |
| | | 01503121 | antidepressant | synthetic | USP, INN, | |
| | | | | | BAN, JAN | |
| | | 01500591 | antipsychotic | synthetic | USP, INN, | |
| | | | | | BAN, JAN | |
| | | 01500592 | anticholinergic, | synthetic | USP, INN, | |
| | | | antiparkinsonian | | BAN, JAN | |
| | | 01500593 | antipruritic | synthetic | USP, INN, | |
| | | | | | BAN, JAN | |
| | | 01500595 | antibacterial | synthetic | USP, INN, | |
| | | | | | BAN, JAN | |
| | | 01503117 | antidepressant | synthetic | USAN, JAN | |
| | | 01500605 | anticholelithogenic; | bear bile | USP, INN, | Hoppe Seyler's Z |
| | | | LD50(rat) 890 | | BAN, JAN | Physiol Chem |
| | | | mg/kg ip | | | 244:181 (1936); |
| | | | | | | Drugs 21:90 |
| | | | | | | (1981); |
| | | | | | | Gastroenterology |
| | | | | | | 91:1007 (1986) |
| | | 01505209 | Angiotensin II | synthetic; | USAN, INN, | |
| | | | inhibitor, | CGP-48933 | BAN | |
| | | | antihypertensive | | | |
| | | 01500607 | antibacterial | Streptomyces | USP, INN, | |
| | | | | orientalis | BAN, JAN | |
| | | 01504171 | antidepressant | synthetic | USAN, INN, BAN | |
| | | 02300307 | adrenegic blocker, | synthetic | USP, INN, | |
| | | | Ca channel | | BAN, JAN | |
| | | | blocker, coronary | | | |
| | | | vasodilator, | | | |
| | | | antiarrhythmic | | | |
| | | 01500663 | alpha adrenergic | Corynanthe | USP | J Chem Soc |
| | | | blocker, mydriatic, | spp | | 1950: 1534; |
| | | | antidepressant | | | Alkaloids 2: 406 |
| | | | | | | (1952); |
| | | | | | | Pharmacol Rev |
| | | | | | | 35: 143 (1983) |
| | | 01502109 | RT transferase | synthetic | USP, INN, | |
| | | | inhibitor, antiviral | | BAN, JAN | |
| | | 01505281 | antimigraine, | synthetic | USAN, INN, | |
| | | | 5HT[1B/1D] | | BAN | |
| | | | agonist | | | |

The 249 compounds were first tested against iron toxicity to human neurons in culture. Neurons were pre-incubated with each compound for 1 h followed by application of FeSO₄. Ferrous iron (25 and 50 μM) is very toxic to neurons, with >80% loss of microtubule-associated protein-2 (MAP2)-labeled neurons by 24 h in most experiments compared to the control condition (Table 2).

TABLE 2

| | Drug % | | Iron % | |
|---|---|---|---|---|
| | control | | control | |
| Name | (mean) | SEM | (mean) | SEM |
| 5-CHLOROINDOLE-2- | 37.30 | 5.87 | 17.33 | 1.12 |
| CARBOXYLIC ACID | | | | |
| ACEBUTOLOL | 49.02 | 13.89 | 26.23 | 8.69 |
| HYDROCHLORIDE | | | | |
| ACETAMINOPHEN | 35.10 | 22.07 | 34.50 | 15.86 |
| ACETAZOLAMIDE | 23.56 | 19.82 | 34.50 | 15.86 |
| ACETYLCYSTEINE | 21.67 | 18.23 | 34.50 | 15.86 |
| ACYCLOVIR | 73.72 | 4.53 | 37.73 | 10.54 |
| ALLOPURINOL | 25.48 | 19.62 | 34.50 | 15.86 |
| ALMOTRIPTAN | 94.44 | 13.68 | 42.76 | 12.68 |
| ALTRETAMINE | 4.20 | 0.12 | 3.19 | 0.14 |
| AMANTADINE | 52.56 | 21.57 | 34.50 | 15.86 |
| HYDROCHLORIDE | | | | |
| AMIKACIN SULFATE | 35.92 | 20.79 | 34.50 | 15.86 |
| AMILORIDE | 37.14 | 21.42 | 34.50 | 15.86 |
| HYDROCHLORIDE | | | | |
| AMIODARONE | 71.35 | 16.08 | 28.43 | 6.81 |
| HYDROCHLORIDE | | | | |
| AMITRIPTYLINE | 34.81 | 17.72 | 34.50 | 15.86 |
| HYDROCHLORIDE | | | | |
| AMLODIPINE BESYLATE | 93.08 | 16.11 | 42.76 | 12.68 |
| AMOXICILLIN | 6.41 | 3.65 | 34.50 | 15.86 |
| AMPHOTERICIN B | 3.41 | 1.33 | 34.50 | 15.86 |
| ANTIPYRINE | 2.11 | 0.66 | 34.50 | 15.86 |
| ASPIRIN | 68.20 | 21.69 | 40.33 | 10.95 |
| ATENOLOL | 43.42 | 12.08 | 14.41 | 3.42 |
| ATORVASTATIN | 68.87 | 4.37 | 37.73 | 10.54 |
| CALCIUM | | | | |
| ATOVAQUONE | 67.74 | 8.78 | 27.13 | 6.35 |
| AZATHIOPRINE | 4.65 | 3.62 | 34.50 | 15.86 |
| AZITHROMYCIN | 56.76 | 20.02 | 37.73 | 10.54 |
| BACITRACIN | 5.04 | 0.51 | 5.03 | 0.78 |
| BACLOFEN | 35.79 | 22.09 | 34.50 | 15.86 |
| BENAZEPRIL | 72.19 | 14.31 | 42.76 | 12.68 |
| HYDROCHLORIDE | | | | |
| BENSERAZIDE | 15.89 | 4.35 | 20.06 | 4.31 |
| HYDROCHLORIDE | | | | |
| BENZTROPINE | 11.43 | 6.78 | 34.50 | 15.86 |
| BETHANECHOL | 15.99 | 9.09 | 34.50 | 15.86 |
| CHLORIDE | | | | |
| BEZAFIBRATE | 35.54 | 14.52 | 14.27 | 4.70 |
| BISACODYL | 93.29 | 8.87 | 20.06 | 4.31 |
| BROMPHENIRAMINE | 79.88 | 7.42 | 35.62 | 8.16 |
| MALEATE | | | | |
| BUDESONIDE | 70.02 | 7.41 | 48.89 | 3.07 |
| BUMETANIDE | 29.38 | 8.82 | 11.56 | 2.85 |
| BUPROPION | 55.54 | 4.03 | 37.73 | 10.54 |
| BUSULFAN | 13.35 | 7.31 | 34.50 | 15.86 |
| CANDESARTAN | 35.48 | 4.57 | 42.76 | 12.68 |
| CILEXTIL | | | | |
| CAPTOPRIL | 35.34 | 7.07 | 25.52 | 4.20 |
| CARBACHOL | 8.87 | 3.78 | 34.50 | 15.86 |
| CARBAMAZEPINE | 13.31 | 4.07 | 34.50 | 15.86 |
| CARVEDILOL TARTRATE | 159.69 | 10.42 | 20.83 | 6.28 |
| CEFACLOR | 89.86 | 3.78 | 9.41 | 3.67 |
| CEFADROXIL | 9.46 | 3.53 | 34.50 | 15.86 |
| CEPHALEXIN | 38.87 | 4.33 | 40.33 | 10.95 |
| CHLORPHENIRAMINE(S) | 52.32 | 9.27 | 20.06 | 4.31 |
| MALEATE | | | | |
| CHLORPROMAZINE | 98.76 | 4.92 | 17.35 | 9.79 |
| CHLORPROPAMIDE | 5.32 | 1.13 | 5.03 | 0.78 |
| CHLORTHALIDONE | 7.66 | 2.15 | 5.03 | 0.78 |
| CIMETIDINE | 34.38 | 11.74 | 13.93 | 4.80 |
| CIPROFLOXACIN | 40.99 | 8.29 | 37.73 | 10.54 |
| CLARITHROMYCIN | 55.09 | 13.17 | 20.83 | 6.28 |
| CLEMASTINE | 6.19 | 0.36 | 5.03 | 0.78 |
| CLINDAMYCIN | 63.15 | 12.24 | 20.06 | 4.31 |
| HYDROCHLORIDE | | | | |
| CLOMIPRAMINE | 107.30 | 11.31 | 18.45 | 4.73 |
| HYDROCHLORIDE | | | | |
| CLONIDINE | 7.47 | 3.10 | 5.03 | 0.78 |
| HYDROCHLORIDE | | | | |
| CLOPIDOGREL SULFATE | 53.53 | 9.02 | 19.15 | 5.36 |
| CLOTRIMAZOLE | 12.36 | 4.00 | 40.33 | 10.95 |
| CLOXACILLIN SODIUM | 28.43 | 10.84 | 12.54 | 3.49 |
| CLOZAPINE | 101.15 | 8.52 | 9.41 | 3.67 |
| COLCHICINE | 3.12 | 0.41 | 5.03 | 0.78 |
| CRESOL | 6.04 | 1.15 | 5.03 | 0.78 |
| CROMOLYN SODIUM | 5.44 | 1.11 | 5.03 | 0.78 |
| CYCLOBENZAPRINE | 98.36 | 12.76 | 35.62 | 8.16 |
| HYDROCHLORIDE | | | | |
| CYCLOPHOSPHAMIDE | 6.39 | 1.16 | 5.03 | 0.78 |
| HYDRATE | | | | |
| CYCLOSPORINE | 11.48 | 0.85 | 17.33 | 1.12 |
| DANAZOL | 4.37 | 0.23 | 5.03 | 0.78 |
| DAPSONE | 18.59 | 5.43 | 7.08 | 2.23 |
| DEQUALINIUM | 10.47 | 0.33 | 17.33 | 1.12 |
| CHLORIDE | | | | |
| DESIPRAMINE | 84.38 | 4.66 | 4.02 | 0.70 |
| HYDROCHLORIDE | | | | |
| DEXTROMETHORPHAN | 3.49 | 0.76 | 5.03 | 0.78 |
| HYDROBROMIDE | | | | |
| DIAZOXIDE | 80.86 | 7.81 | 40.33 | 10.95 |
| DICLOFENAC SODIUM | 5.92 | 1.18 | 5.03 | 0.78 |
| DIFLUNISAL | 4.12 | 0.53 | 5.03 | 0.78 |
| DIGOXIN | 8.91 | 1.80 | 20.06 | 4.31 |
| DILTIAZEM | 86.04 | 11.77 | 35.62 | 8.16 |
| HYDROCHLORIDE | | | | |
| DIMENHYDRINATE | 36.53 | 5.32 | 4.02 | 0.70 |
| DIPHENHYDRAMINE | 74.72 | 6.44 | 4.02 | 0.70 |
| HYDROCHLORIDE | | | | |
| DIPHENYLPYRALINE | 4.61 | 0.96 | 5.03 | 0.78 |
| HYDROCHLORIDE | | | | |
| DIPYRIDAMOLE | 165.07 | 14.85 | 13.26 | 2.59 |
| DISOPYRAMIDE | 4.63 | 1.12 | 5.31 | 0.25 |
| PHOSPHATE | | | | |
| DOXEPIN | 76.91 | 17.10 | 20.02 | 5.71 |
| HYDROCHLORIDE | | | | |
| DOXYCYCLINE | 12.70 | 4.50 | 26.23 | 8.69 |
| HYDROCHLORIDE | | | | |
| DOXYLAMINE | 82.41 | 12.13 | 28.43 | 6.81 |
| SUCCINATE | | | | |
| EDROPHONIUM | 44.00 | 12.26 | 26.23 | 8.69 |
| CHLORIDE | | | | |
| ENALAPRIL MALEATE | 40.97 | 12.64 | 26.23 | 8.69 |
| ERGONOVINE MALEATE | 42.73 | 12.37 | 8.53 | 2.85 |
| ERYTHROMYCIN | 56.71 | 14.49 | 18.45 | 4.73 |
| ESTOLATE | | | | |
| ETHAMBUTOL | 3.72 | 0.94 | 5.31 | 0.25 |
| HYDROCHLORIDE | | | | |
| ETHOSUXIMIDE | 74.29 | 18.77 | 35.62 | 8.16 |
| ETODOLAC | 34.42 | 10.33 | 13.93 | 4.80 |
| EZETIMIBE | 50.46 | 10.96 | 42.76 | 12.68 |
| FAMCICLOVIR | 91.00 | 12.00 | 42.76 | 12.68 |
| FAMOTIDINE | 25.23 | 9.73 | 13.93 | 4.80 |
| FENOFIBRATE | 24.43 | 7.32 | 13.93 | 4.80 |
| FLUNARIZINE | 126.36 | 9.16 | 9.86 | 2.61 |
| HYDROCHLORIDE | | | | |
| FLUOXETINE | 81.41 | 11.56 | 35.62 | 8.16 |
| FLUPHENAZINE | 12.13 | 4.32 | 25.52 | 4.20 |
| HYDROCHLORIDE | | | | |
| FLURBIPROFEN | 4.63 | 0.44 | 5.31 | 0.25 |
| FOSFOMYCIN | 31.24 | 9.29 | 11.56 | 2.85 |
| FUROSEMIDE | 3.96 | 0.74 | 5.31 | 0.25 |
| GEMFIBROZIL | 5.05 | 0.73 | 5.31 | 0.25 |
| GLICLAZIDE | 47.31 | 5.08 | 37.73 | 10.54 |
| GLYBURIDE | 45.24 | 1.39 | 48.89 | 3.07 |
| GUAIFENESIN | 3.28 | 0.30 | 5.31 | 0.25 |
| HALOPERIDOL | 6.12 | 1.05 | 5.31 | 0.25 |
| HEXYLRESORCINOL | 71.52 | 8.88 | 20.06 | 4.31 |
| HYDRALAZINE | 10.15 | 3.05 | 2.76 | 0.97 |
| HYDROCHLORIDE | | | | |
| HYDROCHLOROTHIAZIDE | 2.55 | 0.37 | 5.31 | 0.25 |
| HYDROXYCHLOROQUINE | 75.87 | 15.95 | 35.62 | 8.16 |
| SULFATE | | | | |
| HYDROXYUREA | 3.31 | 0.45 | 5.31 | 0.25 |
| HYDROXYZINE | 4.01 | 1.05 | 5.31 | 0.25 |
| PAMOATE | | | | |
| IBUPROFEN | 2.48 | 0.52 | 2.96 | 0.78 |
| IMIPRAMINE | 106.49 | 7.76 | 13.26 | 2.59 |
| HYDROCHLORIDE | | | | |
| INDAPAMIDE | 126.12 | 2.79 | 1.58 | 0.63 |
| INDOMETHACIN | 4.52 | 1.34 | 2.96 | 0.78 |
| IPRATROPIUM BROMIDE | 63.39 | 20.68 | 40.33 | 10.95 |
| IRBESARTAN | 60.93 | 3.13 | 42.76 | 12.68 |
| ISONIAZID | 2.41 | 0.55 | 2.96 | 0.78 |
| ISOSORBIDE DINITRATE | 1.92 | 0.38 | 2.96 | 0.78 |
| KETOCONAZOLE | 108.35 | 2.80 | 1.58 | 0.63 |
| KETOPROFEN | 44.26 | 11.34 | 14.41 | 3.42 |
| KETOROLAC | 52.39 | 14.15 | 35.62 | 8.16 |
| TROMETHAMINE | | | | |
| KETOTIFEN FUMARATE | 1.77 | 0.83 | 25.52 | 4.20 |
| LABETALOL | 54.37 | 11.87 | 23.26 | 5.81 |
| HYDROCHLORIDE | | | | |
| LACTULOSE | 80.82 | 19.43 | 40.33 | 10.95 |
| LANSOPRAZOLE | 63.87 | 1.81 | 37.73 | 10.54 |
| LEUCOVORIN CALCIUM | 24.84 | 21.16 | 2.96 | 0.78 |
| LEVODOPA | 81.18 | 3.71 | 26.23 | 8.69 |
| LEVOFLOXACIN | 56.44 | 8.21 | 37.73 | 10.54 |
| LIOTHYRONINE SODIUM | 141.46 | 10.60 | 12.35 | 2.03 |
| LISINOPRIL | 48.67 | 16.98 | 26.23 | 8.69 |
| LOPERAMIDE | 55.50 | 12.86 | 20.02 | 5.71 |
| HYDROCHLORIDE | | | | |
| LORATADINE | 44.26 | 3.86 | 37.73 | 10.54 |
| LOSARTAN | 35.45 | 4.03 | 42.76 | 12.68 |
| LOVASTATIN | 32.18 | 10.01 | 37.73 | 10.54 |
| LOXAPINE SUCCINATE | 65.91 | 8.00 | 40.33 | 10.95 |
| MAPROTILINE | 0.61 | 0.29 | 2.96 | 0.78 |
| HYDROCHLORIDE | | | | |
| MEBENDAZOLE | 2.48 | 0.44 | 25.52 | 4.20 |
| MEFENAMIC ACID | 57.21 | 4.90 | 40.33 | 10.95 |
| MEFLOQUINE | 47.01 | 9.07 | 12.35 | 2.03 |
| MELOXICAM | 59.46 | 11.56 | 37.73 | 10.54 |
| MEMANTINE | 53.24 | 12.40 | 9.41 | 3.67 |
| HYDROCHLORIDE | | | | |
| MERCAPTOPURINE | 1.73 | 0.37 | 2.96 | 0.78 |
| METHAZOLAMIDE | 54.29 | 17.70 | 35.62 | 8.16 |
| METHENAMINE | 1.94 | 0.04 | 2.96 | 0.78 |
| METHOCARBAMOL | 0.84 | 0.22 | 2.96 | 0.78 |
| METHOTREXATE | 43.34 | 19.47 | 48.59 | 19.48 |
| METHOXSALEN | 59.05 | 18.46 | 48.59 | 19.48 |
| METHYLDOPA | 101.58 | 5.66 | 24.35 | 12.85 |
| METOCLOPRAMIDE | 37.87 | 2.00 | 48.59 | 19.48 |
| HYDROCHLORIDE | | | | |
| METOLAZONE | 68.98 | 14.60 | 26.08 | 5.27 |
| METOPROLOL | 71.55 | 16.46 | 24.35 | 12.85 |
| TARTRATE | | | | |
| METRONIDAZOLE | 27.08 | 2.88 | 48.59 | 19.48 |
| MIDODRINE | 53.69 | 6.41 | 35.62 | 8.16 |
| HYDROCHLORIDE | | | | |
| MINOXIDIL | 33.66 | 3.31 | 48.59 | 19.48 |
| MITOXANTHRONE | 52.54 | 4.13 | 10.26 | 2.72 |
| HYDROCHLORIDE | | | | |
| MODAFINIL | 43.94 | 14.98 | 26.23 | 8.69 |
| MOXIFLOXACIN | 51.59 | 4.29 | 37.73 | 10.54 |
| HYDROCHLORIDE | | | | |
| MYCOPHENOLIC ACID | 45.69 | 11.70 | 12.07 | 3.12 |
| NABUMETONE | 48.91 | 8.07 | 35.62 | 8.16 |
| NADOLOL | 52.39 | 11.65 | 35.62 | 8.16 |
| NALOXONE | 69.47 | 3.48 | 48.59 | 19.48 |
| HYDROCHLORIDE | | | | |
| NALTREXONE | 39.55 | 4.02 | 35.62 | 8.16 |
| HYDROCHLORIDE | | | | |
| NAPROXEN(+) | 25.64 | 4.24 | 48.59 | 19.48 |
| NEOSTIGMINE BROMIDE | 44.83 | 5.13 | 48.59 | 19.48 |
| NIFEDIPINE | 14.68 | 1.31 | 48.59 | 19.48 |
| NILUTAMIDE | 48.72 | 14.62 | 35.62 | 8.16 |
| NIMODIPINE | 62.84 | 14.99 | 37.73 | 10.54 |
| NITROFURANTOIN | 17.84 | 1.31 | 48.59 | 19.48 |
| NORFLOXACIN | 13.59 | 2.17 | 48.59 | 19.48 |
| NORTRIPTYLINE | 18.16 | 2.89 | 48.59 | 19.48 |
| NYLIDRIN | 50.07 | 12.07 | 22.92 | 8.49 |
| HYDROCHLORIDE | | | | |
| OLIVEESARTAN | 55.85 | 6.20 | 42.76 | 12.68 |
| MEDOXOMIL | | | | |
| ORLISTAT | 1.00 | 0.10 | 42.76 | 12.68 |
| ORPHENADRINE | 54.50 | 11.76 | 22.92 | 8.49 |
| CITRATE | | | | |
| OXCARBAZEPINE | 38.18 | 5.58 | 42.76 | 12.68 |
| PAROMOMYCIN | 35.29 | 3.13 | 17.33 | 1.12 |
| SULFATE | | | | |
| PENTOXIFYLLINE | 66.47 | 12.78 | 35.62 | 8.16 |
| PERICIAZINE | 81.97 | 11.21 | 19.15 | 5.36 |
| PERINDOPRIL | 49.36 | 15.80 | 26.23 | 8.69 |
| ERBUMINE | | | | |
| PERPHENAZINE | 78.78 | 17.35 | 18.45 | 4.73 |
| PHENAZOPYRIDINE | 101.46 | 8.17 | 24.35 | 12.85 |
| HYDROCHLORIDE | | | | |
| PHENELZINE SULFATE | 46.68 | 8.55 | 48.59 | 19.48 |
| PHENYTOIN SODIUM | 30.13 | 8.56 | 48.59 | 19.48 |
| PIMOZIDE | 31.41 | 0.74 | 17.33 | 1.12 |
| PINDOLOL | 62.64 | 22.61 | 40.33 | 10.95 |
| PIOGLITAZONE | 84.58 | 14.90 | 42.76 | 12.68 |
| HYDROCHLORIDE | | | | |
| PIROXICAM | 36.16 | 6.65 | 40.33 | 10.95 |
| POTASSIUM | 44.46 | 7.50 | 20.06 | 4.31 |
| p-AMINOBENZOATE | | | | |
| PRAVASTATIN SODIUM | 40.51 | 11.81 | 26.23 | 8.69 |
| PREGABALIN | 47.81 | 15.36 | 26.23 | 8.69 |
| PRIMAQUINE | 89.07 | 4.70 | 24.35 | 12.85 |
| DIPHOSPHATE | | | | |
| PRIMIDONE | 45.23 | 5.07 | 40.33 | 10.95 |
| PROBENECID | 71.46 | 10.59 | 40.33 | 10.95 |
| PROCAINAMIDE | 64.28 | 12.63 | 40.33 | 10.95 |
| HYDROCHLORIDE | | | | |
| PROCHLORPERAZINE | 4.88 | 0.44 | 20.06 | 4.31 |
| EDISYLATE | | | | |
| PROCYCLIDINE | 95.64 | 22.09 | 40.33 | 10.95 |
| HYDROCHLORIDE | | | | |
| PROMETHAZINE | 105.40 | 7.03 | 7.52 | 3.06 |
| HYDROCHLORIDE | | | | |
| PROPAFENONE | 51.34 | 6.56 | 37.73 | 10.54 |
| HYDROCHLORIDE | | | | |
| PROPRANOLOL | 66.49 | 4.12 | 40.33 | 10.95 |
| HYDROCHLORIDE (+/−) | | | | |
| PROPYLTHIOURACIL | 35.91 | 2.49 | 16.53 | 1.48 |
| PSEUDOEPHEDRINE | 26.74 | 3.16 | 14.94 | 2.65 |
| HYDROCHLORIDE | | | | |
| PYRANTEL PAMOATE | 34.17 | 3.87 | 12.67 | 2.66 |
| PYRAZINAMIDE | 67.20 | 5.41 | 48.89 | 3.07 |
| PYRIDOSTIGMINE | 35.78 | 3.60 | 17.33 | 1.12 |
| BROMIDE | | | | |
| QUINAPRIL | 41.55 | 4.83 | 17.33 | 1.12 |
| HYDROCHLORIDE | | | | |
| QUININE SULFATE | 21.34 | 4.35 | 14.94 | 2.65 |
| RANITIDINE | 40.18 | 8.86 | 17.33 | 1.12 |
| RIFAMPIN | 95.53 | 5.13 | 7.52 | 3.06 |
| RIFAXIMIN | 53.80 | 18.27 | 26.23 | 8.69 |
| RILUZOLE | 56.54 | 15.74 | 26.23 | 8.69 |
| ROSIGLITAZONE | 77.63 | 8.97 | 42.76 | 12.68 |
| ROSUVASTATIN | 35.43 | 3.92 | 42.76 | 12.68 |
| SERTRALINE | 24.23 | 4.43 | 42.76 | 12.68 |
| HYDROCHLORIDE | | | | |
| SILDENAFIL | 49.31 | 2.47 | 37.73 | 10.54 |
| SPIRAMYCIN | 63.97 | 11.40 | 37.73 | 10.54 |
| SPIRONOLACTONE | 37.11 | 9.86 | 8.83 | 2.55 |
| SULFAMETHOXAZOLE | 16.23 | 2.22 | 14.94 | 2.65 |
| SULFASALAZINE | 23.36 | 2.42 | 14.94 | 2.65 |
| SULFINPYRAZONE | 46.51 | 1.33 | 48.89 | 3.07 |
| SULFISOXAZOLE | 38.28 | 12.78 | 26.23 | 8.69 |
| SULINDAC | 34.51 | 7.87 | 11.70 | 2.97 |
| TENOXICAM | 25.92 | 3.53 | 17.33 | 1.12 |
| TETRACYCLINE | 25.04 | 6.42 | 11.70 | 2.97 |
| HYDROCHLORIDE | | | | |
| THEOPHYLLINE | 23.29 | 5.80 | 14.94 | 2.65 |
| THIOGUANINE | 21.73 | 3.99 | 14.94 | 2.65 |
| THIOTHIXENE | 6.80 | 1.01 | 26.23 | 8.69 |
| TIMOLOL MALEATE | 11.07 | 1.14 | 14.94 | 2.65 |
| TOLBUTAMIDE | 9.09 | 2.06 | 14.94 | 2.65 |
| TOLFENAMIC ACID | 40.26 | 2.90 | 17.33 | 1.12 |
| TOPIRAMATE | 46.07 | 15.57 | 26.23 | 8.69 |
| TRANDOLAPRIL | 72.30 | 6.44 | 42.76 | 12.68 |
| TRANEXAMIC ACID | 36.26 | 2.56 | 17.33 | 1.12 |
| TRANYLCYPROMINE | 21.59 | 3.15 | 14.94 | 2.65 |
| SULFATE | | | | |
| TRAZODONE | 25.93 | 8.38 | 10.26 | 2.72 |
| HYDROCHLORIDE | | | | |
| TRIFLUOPERAZINE | 4.42 | 2.01 | 14.94 | 2.65 |
| HYDROCHLORIDE | | | | |
| TRIHEXYPHENIDYL | 30.57 | 5.61 | 14.94 | 2.65 |
| HYDROCHLORIDE | | | | |
| TRIMEPRAZINE | 73.31 | 7.34 | 7.52 | 3.06 |
| TARTRATE | | | | |
| TRIMETHOPRIM | 13.96 | 3.09 | 14.94 | 2.65 |
| TRIMIPRAMINE | 88.62 | 11.61 | 18.45 | 4.73 |
| MALEATE | | | | |
| URSODIOL | 24.62 | 2.10 | 14.94 | 2.65 |
| VALSARTAN SODIUM | 64.68 | 10.94 | 42.76 | 12.68 |
| VANCOMYCIN | 11.70 | 8.21 | 12.95 | 5.13 |
| HYDROCHLORIDE | | | | |
| VENLAFAXINE | 72.52 | 10.20 | 37.73 | 10.54 |
| VERAPAMIL | 71.08 | 13.71 | 40.33 | 10.95 |
| HYDROCHLORIDE | | | | |
| YOHIMBINE | 100.09 | 4.40 | 9.41 | 3.67 |
| HYDROCHLORIDE | | | | |
| ZIDOVUDINE [AZT] | 66.49 | 7.87 | 35.62 | 8.16 |
| ZOLMITRIPTAN | 54.88 | 8.92 | 42.76 | 12.68 |

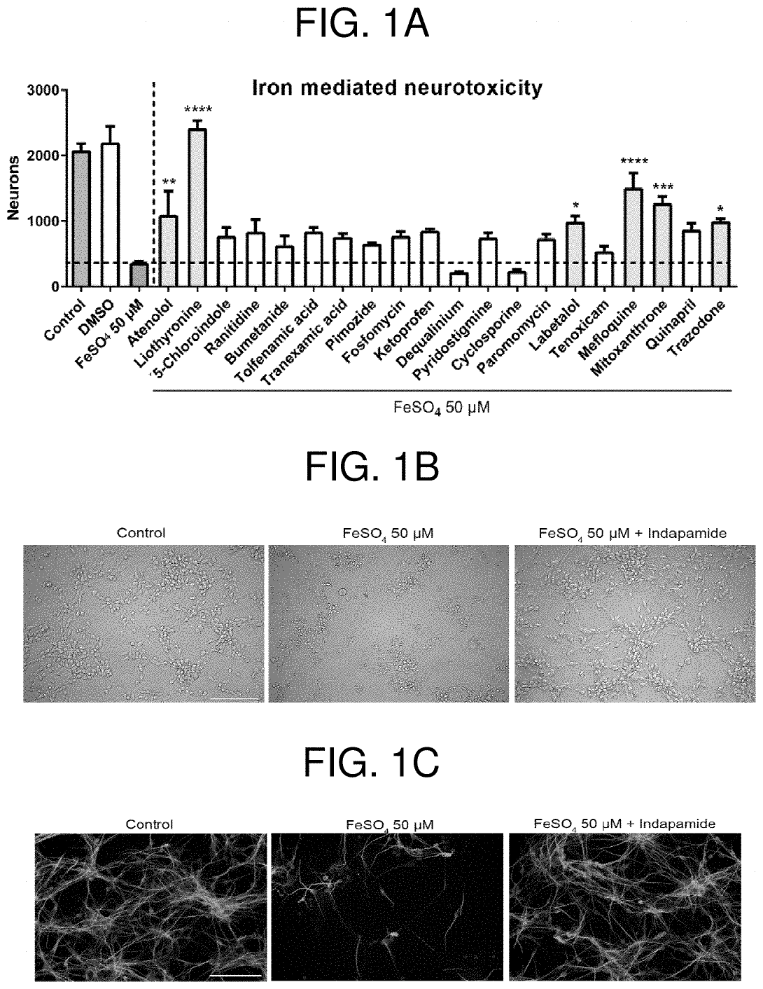

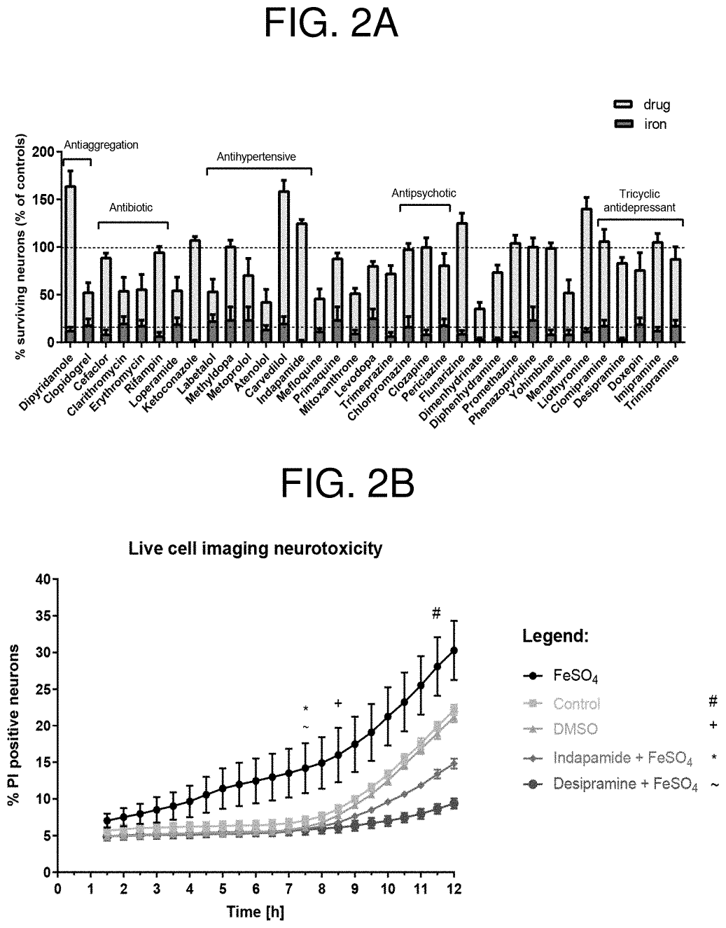

An example of iron toxicity and a drug screen is shown in FIG. 1. Of all drugs tested, 35 compounds showed statistically significant protection from FeSO₄-mediated neurotoxicity (FIG. 2a). Of these, antipsychotics such as clozapine or periciazine, and tricyclic antidepressants such as clomipramine or desipramine, exhibited strong protection, as shown after normalization across at least 2-4 experiments (n of 4 wells of cells per experiment per test condition) to the number of neurons of the respective control conditions (FIG. 2A). For example, while the average loss of neurons over 24 h in response to FeSO₄ was 85.5% (i.e. 14.5% of surviving neurons compared to 100% of controls), clomipramine at 10 pM completely prevented neuronal loss (107.3% of controls). Other categories of medications with neuroprotective actions against iron included anti-hypertensives and some antibiotics. We note that minocycline, an antibiotic that reduces the conversion of a first demyelinating event to clinically definite multiple sclerosis in a Phase 3 clinical trial was not included in the 1040 compounds; in a separate study, we find minocycline to completely prevent iron neurotoxicity as well (Faissner S, et al. Unexpected additive effects of minocycline and hydroxychloroquine in models of multiple sclerosis: Prospective combination treatment for progressive disease? Multiple sclerosis (Houndmills, Basingstoke, England), 1352458517728811 (2017).

Live cell imaging over 12 h supported the neuroprotective effects of drugs. We selected indapamide and desipramine for live imaging studies. FIG. 2b shows that while the number of neurons with intracellular propidium iodide (PI), a dye that leaks across a compromised plasma membrane, in response to FeSO₄ exposure increases progressively over 12 h, this was significantly attenuated by indapamide and desipramine.

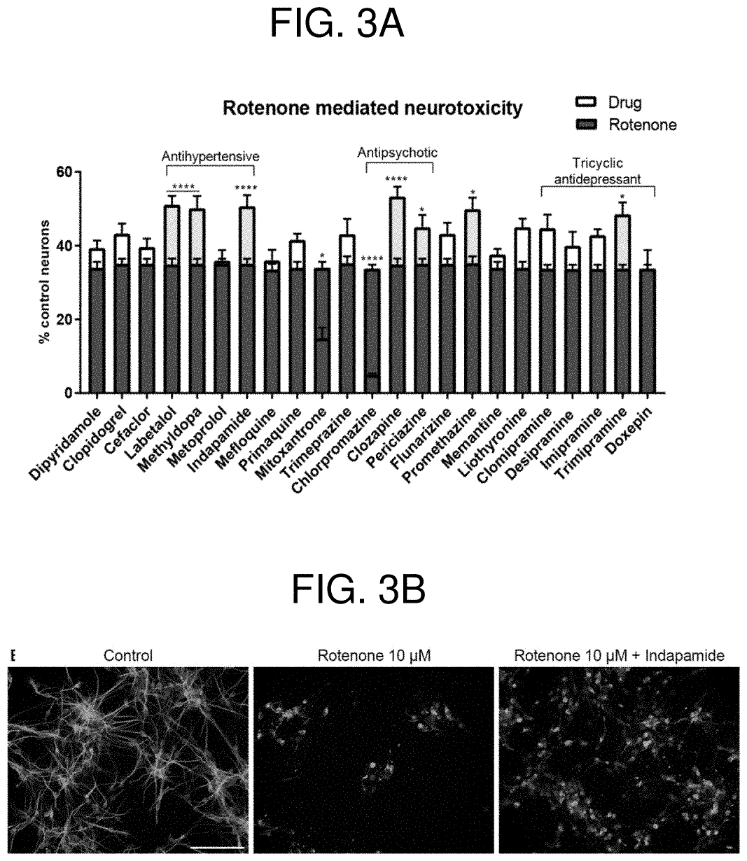

The 35 hits were further narrowed concerning their ability to cross the blood-brain-barrier according to drugbank.ca, their side effect profile and tolerability. Although antipsychotics are not well tolerated they were further included in the screening due to their good blood-brain-barrier penetrance. Out of these, a group of 23 compounds was chosen for their ability to prevent mitochondrial damage using rotenone, which inhibits the electron transfer from complex I of the respiratory chain to ubiquinone. Rotenone induced strong neurotoxicity to neurons (FIG. 3). The tricyclic antidepressant trimipramine, the antipsychotics clozapine and periciazine, promethazine and the anti-hypertensives labetalol, methyldopa and indapamide reduced neurotoxicity while clomipramine trended towards a protective activity (FIG. 3A). The effect size of rescue by medications was, however, small. Of note, rotenone induced marked morphological neuronal changes with retraction of neurites (FIG. 3B).

Hydroxyl Radical Scavenging Capacity of Medications

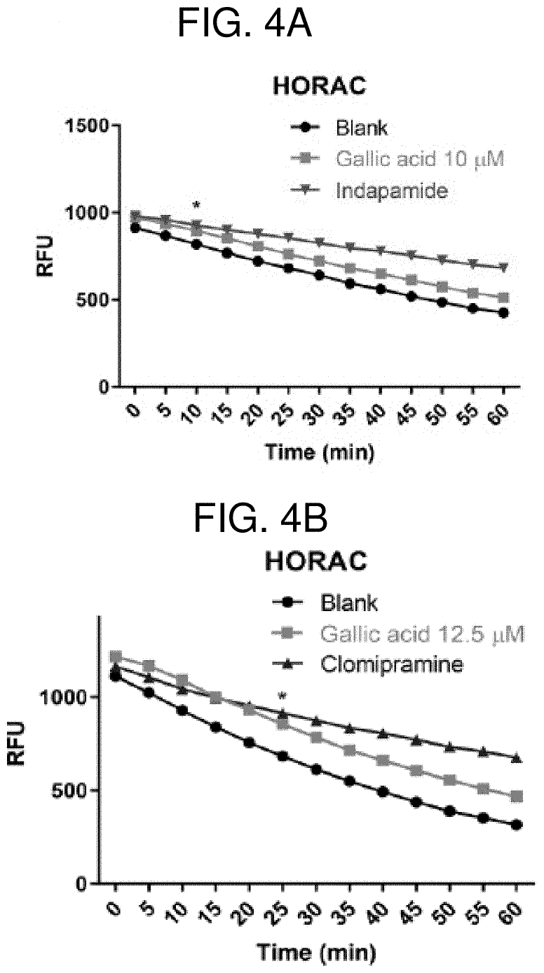

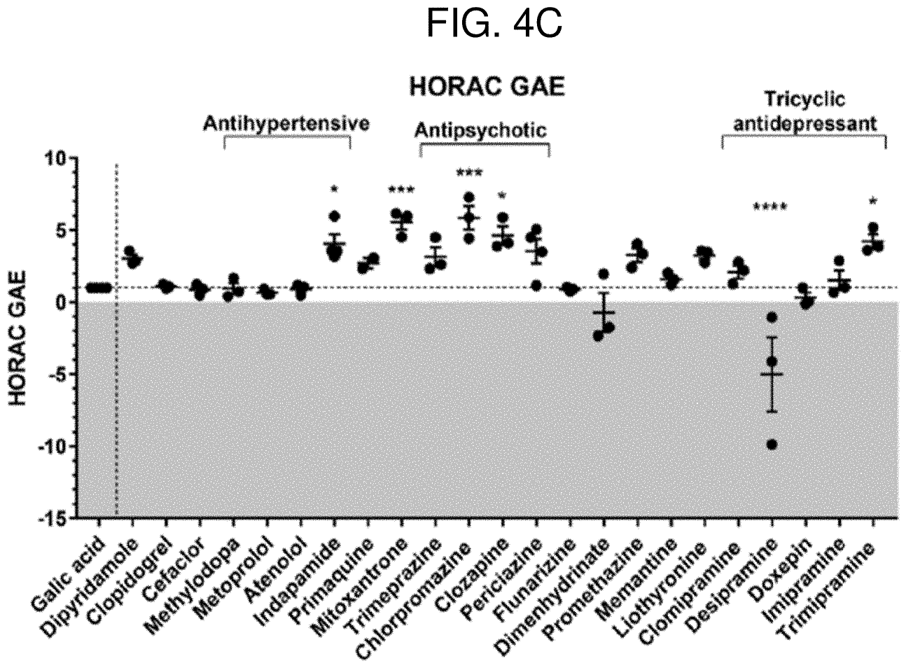

The biochemical cell free hydroxyl radical antioxidant capacity (HORAC) assay investigates the prevention of hydroxyl radical mediated oxidation of to fluorescein in comparison to the strong anti-oxidant gallic acid. The generation of hydroxyl radicals by a cobalt-driven Fenton-like reaction oxidizes fluorescein with progressive loss of fluorescence. The presence of an anti-oxidant reduces the loss of fluorescence over time. As noted in FIG. 4A, gallic acid reduced the loss of fluorescence (upward shift) compared to a blank Fenton-driven reaction that is without anti-oxidant, while indapamide has an even higher activity.

We compared the area under the curve of test compounds to that elicited by gallic acid to obtain the gallic acid equivalent (GAE). A GAE of 1 represents hydroxyl radical scavenging capacity similar to that of gallic acid, while a compound without anti-oxidant activity would produce a GAE close to 0. Some of the compounds tested exhibited stronger anti-oxidative properties than gallic acid with HORAC-GAEs >1 (FIG. 4C). These included indapamide (mean HORAC-GAE 4.1; p<0.05; one-way analysis of variance (ANOVA) with Dunnett's multiple comparisons test as post-hoc analysis vs. gallic acid), mitoxantrone (5.6; p<0.001), chlorpromazine (5.9; p<0.001), clozapine (4.6; p<0.05) and trimipramine (4.2; p<0.05). Although not statistically significant compared to gallic acid, clomipramine had a HORAC-GAE of 2.1. Regarding the comparison to the blank situation (i.e. no anti-oxidant present), there was a significant upward shift by clomipramine of the slope over 60 min (p<0.0001) (FIG. 4b). Thus, although clomipramine lacked significance against the strong anti-oxidative gallic acid, the compound exhibited strong anti-oxidative effects against the blank situation (in the absence of any anti-oxidant). Interestingly, the tricyclic antidepressant desipramine had strong oxidative effects (HORAC-GAE -5.00; p<0.0001).

Proliferation of T-Lymphocytes is Reduced by Antidepressants

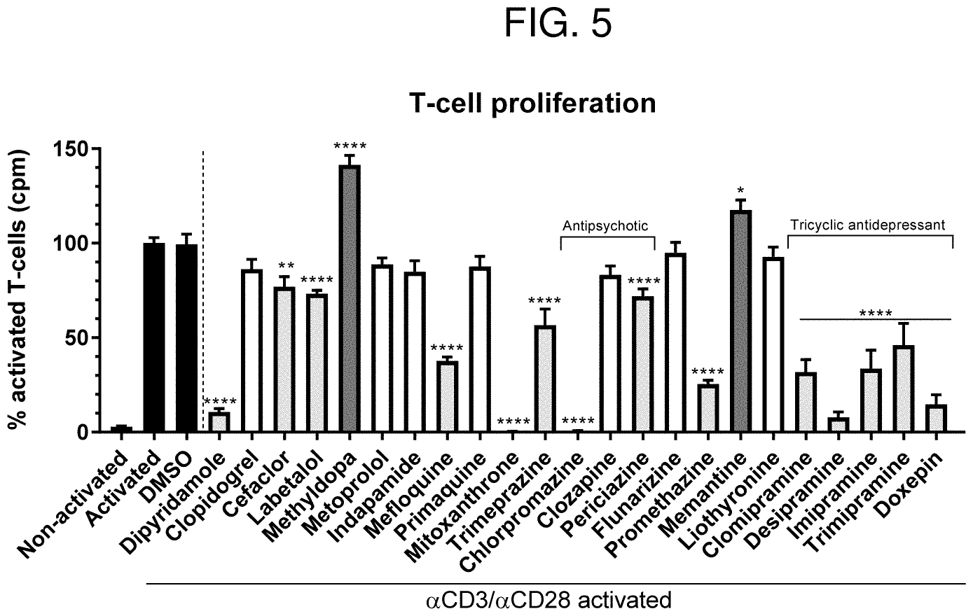

We tested the capacity of compounds to affect T-cell proliferation (FIG. 5). Splenocytes activated by anti-CD3/anti-CD28 to trigger the proliferation of T-cells had reduced incorporation of ³[H]-thymidine upon treatment with dipyridamole (mean reduction 89.3%; p<0.0001; one-way ANOVA with Dunnett's multiple comparisons test as post-hoc analysis compared to activated splenocytes), cefaclor (23%; p<0.01), labetalol (26.8%, p<0.0001 for this and subsequent compounds listed here), mefloquine (62.3%), mitoxantrone (99.7%), trimeprazine (43.3%), chlorpromazine (99.4%), periciazine (28%), promethazine (74.6%), clomipramine (68.2%), desipramine (92.2%), imipramine (66.4%), trimipramine (54%) and doxepin (85.3%, all p<0.0001). Of note, methyldopa and memantine increased proliferation (methyldopa 41.4%, p<0.0001; memantine 17.5%, p<0.05). Mitoxantrone and chlorpromazine, however, had toxic effects (data not shown).

Focus on Clomipramine In Vitro and in Acute and Chronic EAE

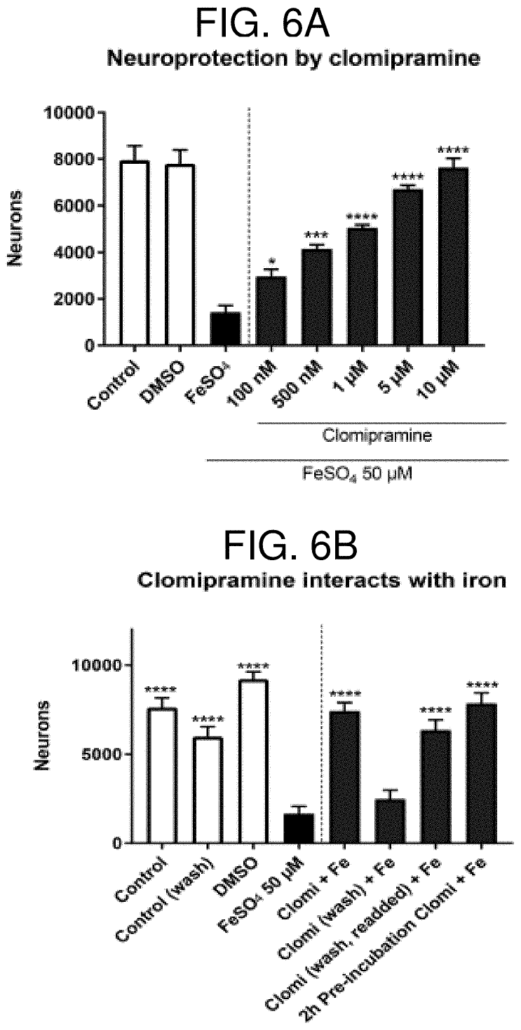

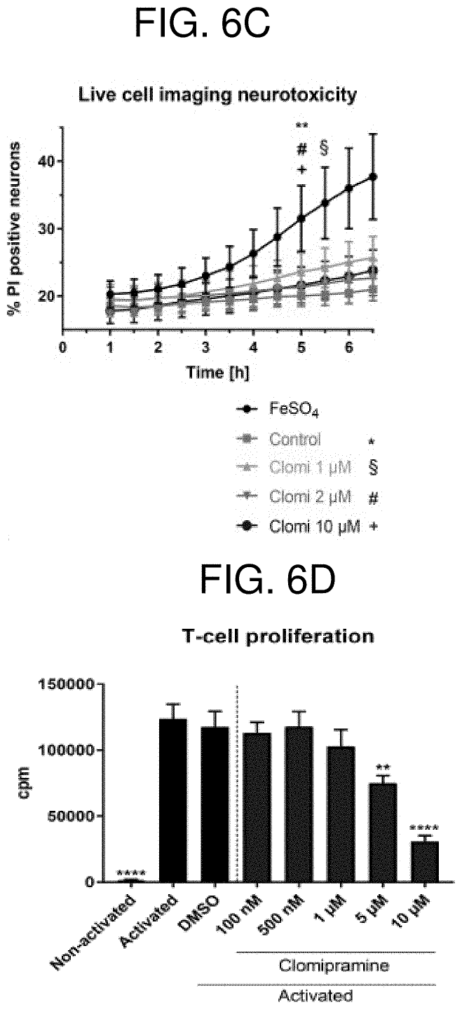

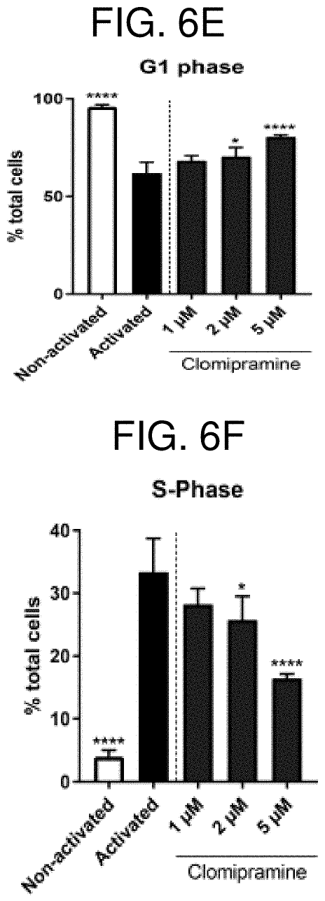

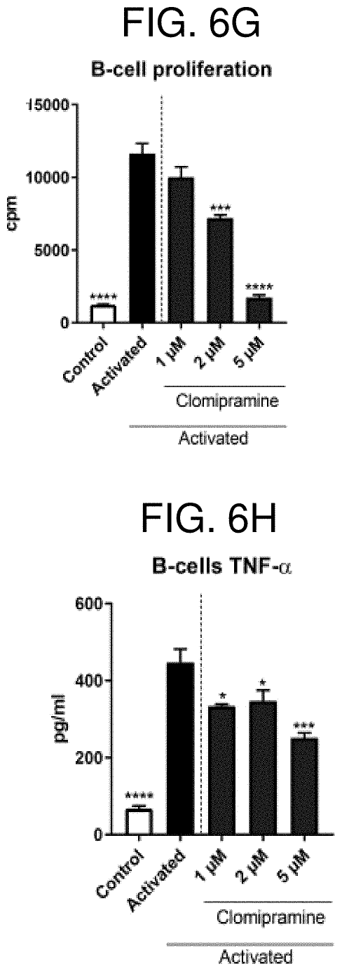

We selected clomipramine for further study as it is a well-tolerated anti-depressant and crosses the blood-brain barrier very well (drugbank.ca). Moreover, in our assays, clomipramine showed strong effects against iron mediated neurotoxicity (mean % anti-microtubule-associated protein-2 (MAP-2) positive cells normalized to control of 107.3%, representing complete protection against iron toxicity)(FIG. 2), had anti-oxidative properties (HORAC-GAE 2.1 where the effect of the anti-oxidant gallic acid is normalized at 1)(FIG. 4), and reduced T-lymphocyte proliferation (by 68.2%) (FIG. 5). We began with a concentration response with the intent of investigating lower concentrations since plasma concentration in human of clomipramine as an anti-depressant average 122 ng/ml (387 nM) (Rodriguez de la Torre et al., 2001), but can peak to more than 600 nM in some individuals (Thoren et al., 1980). FIG. 6A shows that clomipramine had a progressive significant increase in neuroprotection against iron toxicity from 100 nM. The effect was mediated in part by chelation with iron, as washing away clomipramine from neurons led to cell death, while pre-incubation with iron before application to neurons totally preserved neuronal viability (FIG. 6B). We were able to observe the protection by clomipramine in a live-cell imaging study, in which the increasing number of PI-positive neurons over time in response to iron was attenuated by clomipramine (FIG. 6C).

T-lymphocyte proliferation was reduced in a concentration-dependent manner by clomipramine but significant reduction occurred only from 5 μM (p<0.01; one-way ANOVA with Dunnett's multiple comparisons test as post-hoc analysis compared to activated T-lymphocytes)) (FIG. 6D). This was reflected by a cell cycle arrest with more cells in G1 (p<0.05) and less in the S-phase (p<0.05) from 2 μM (FIG. 6E, F).

Due to the growing knowledge about the importance of B-cell follicular structures for progressive multiple sclerosis (Romme Christensen et al., 2013; Magliozzi R, et al. Meningeal B-cell follicles in secondary progressive multiple sclerosis associate with early onset of disease and severe cortical pathology. Brain 130, 1089-1104 (2007)), we sought to evaluate the effect of clomipramine on B-cell activation. BCR/anti-CD4OL/IL-4 activation of B-cells increased their proliferation and production of TNF-α (FIG. 6G, H) and these were reduced in a concentration-dependent manner by clomipramine from 2 μM.

We then investigated clomipramine in acute EAE. Therapy with clomipramine from day 5 after induction of MOG-EAE delayed onset of clinical signs by 2 days with a significantly better early disease course between days 11 and 18 (FIG. 7A), which was reflected in an overall lower burden of disability (FIG. 7B). However, eventually, clomipramine treated animals succumbed to EAE and increased disability (FIG. 7A)

We then sought to investigate whether initiation of treatment from the the day of MOG-induction could improve the outcome of EAE. Remarkably, early treatment initiation completely suppressed the manifestations of clinical signs (FIG. 8A). While most animals in the vehicle group had a high disease burden, as shown by the sum of scores for each individual animal (FIG. 8B) and weight loss (FIG. 8C), this was profoundly ameliorated in treated mice over the course of study. PCR analyses of the spinal cord revealed that the significant elevation in vehicle-EAE mice of transcripts encoding Ifng, Tnfa, II-17 and CcI2 were abrogated in clomipramine-EAE mice (FIG. 8D).

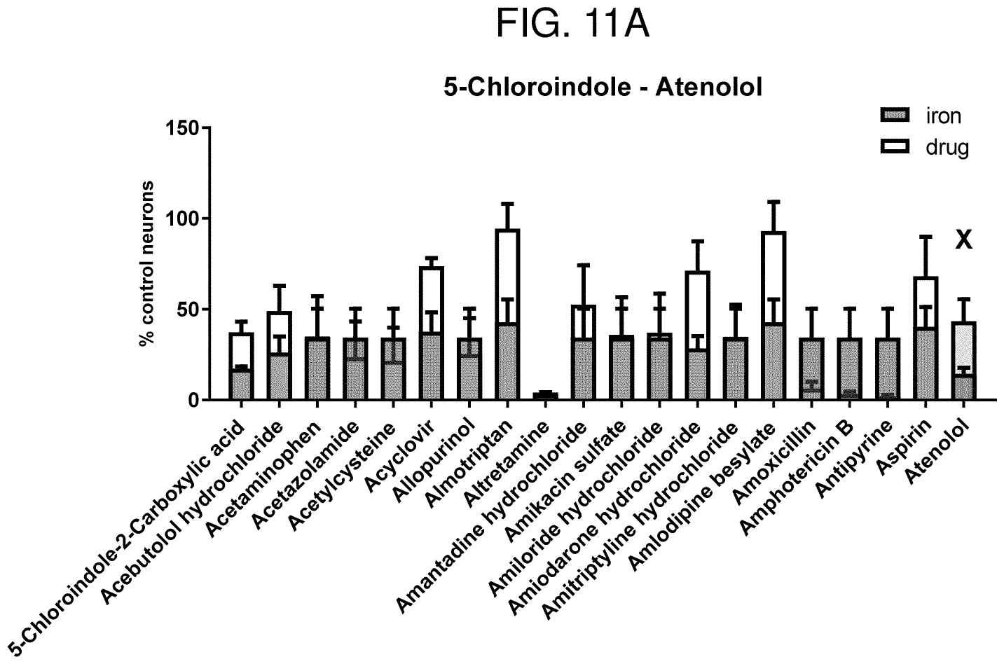

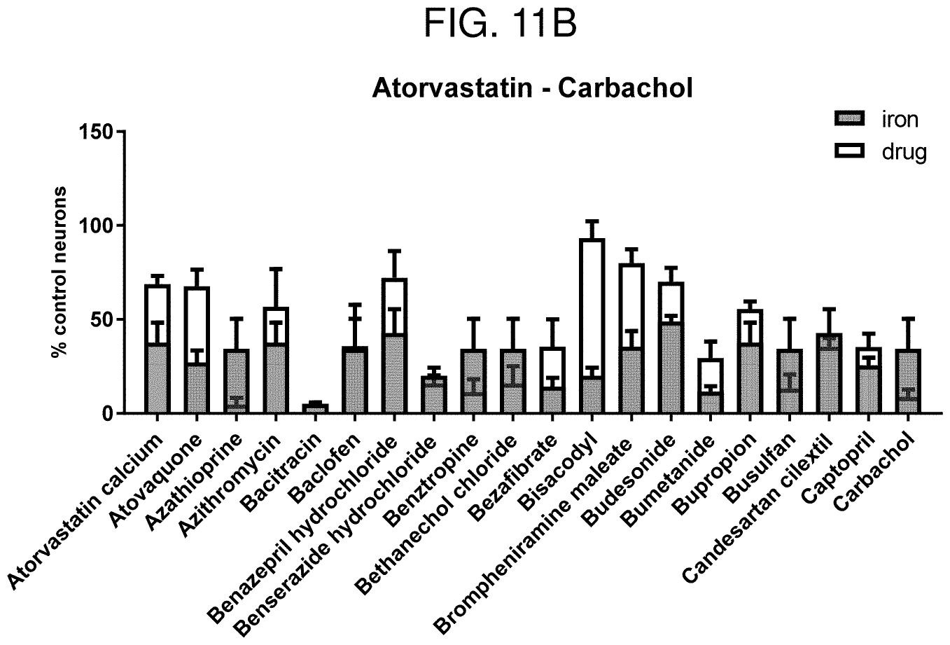

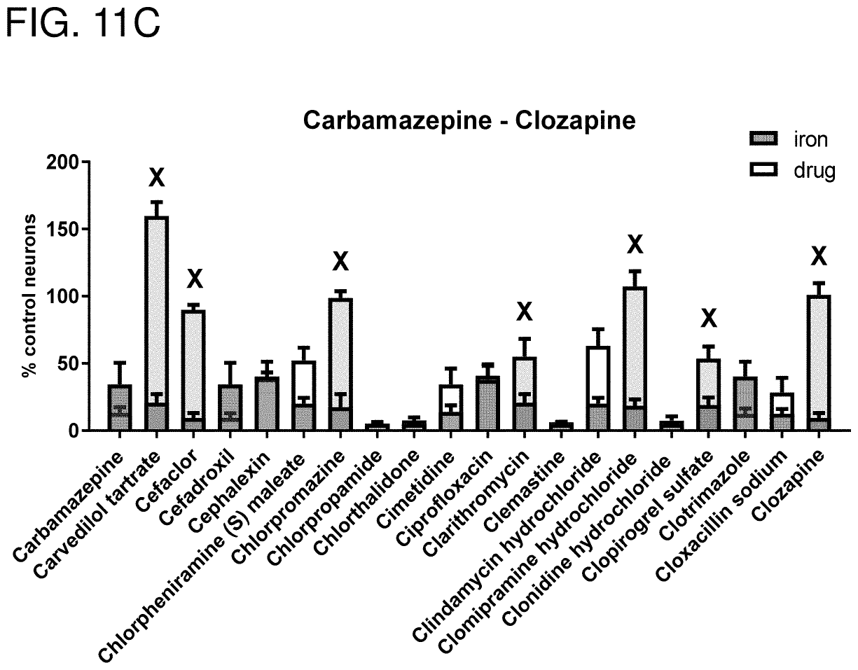

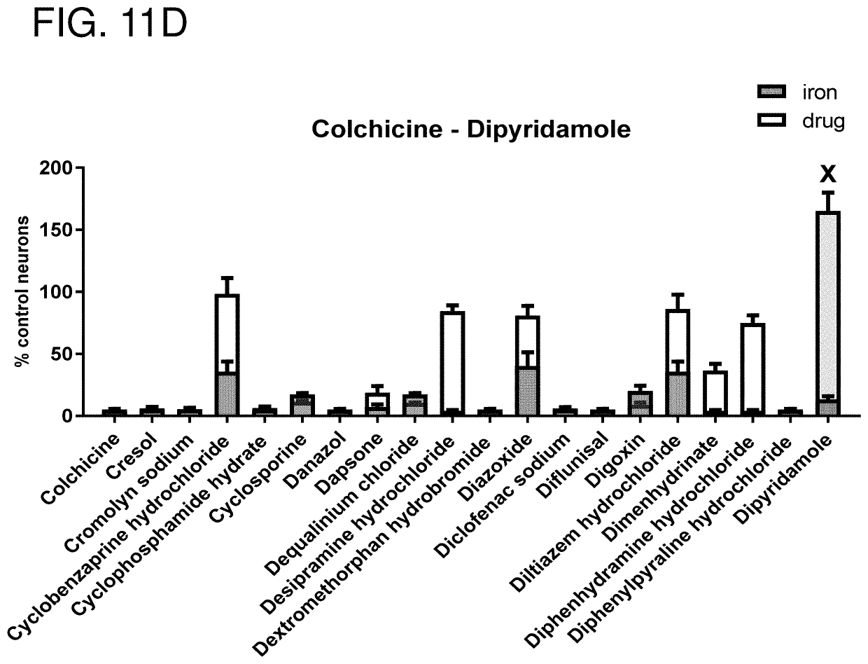

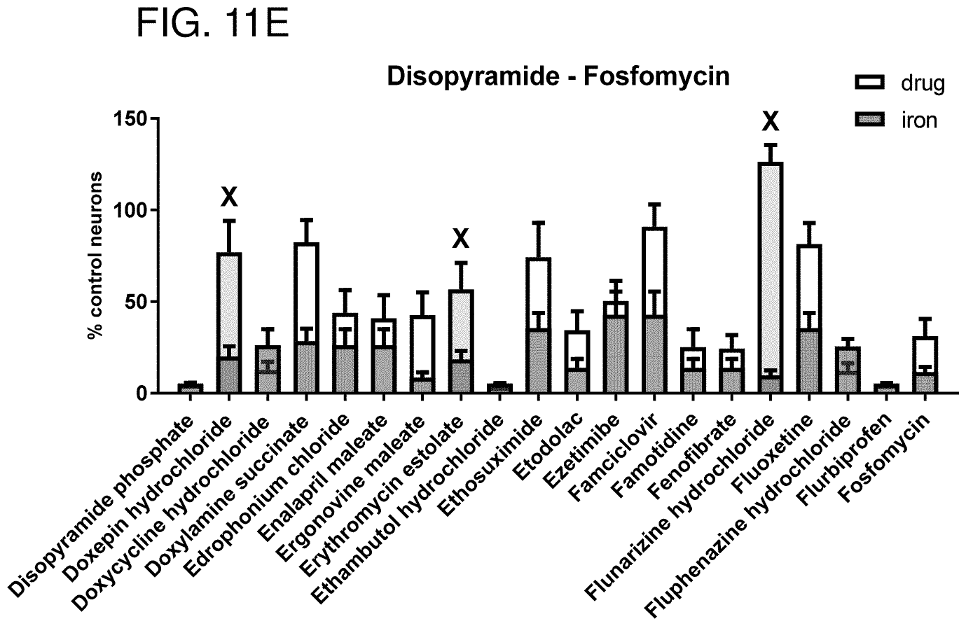

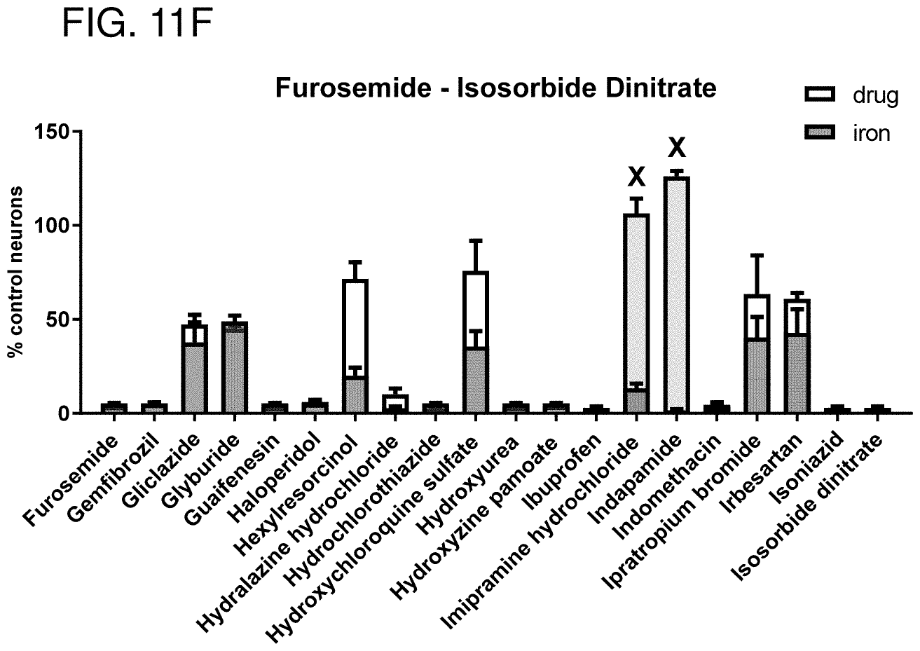

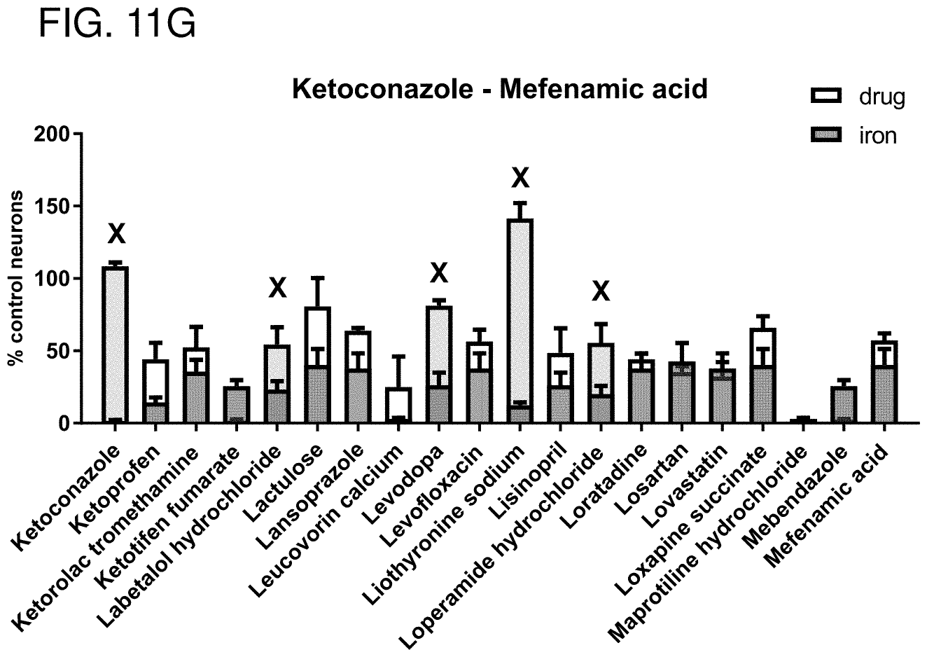

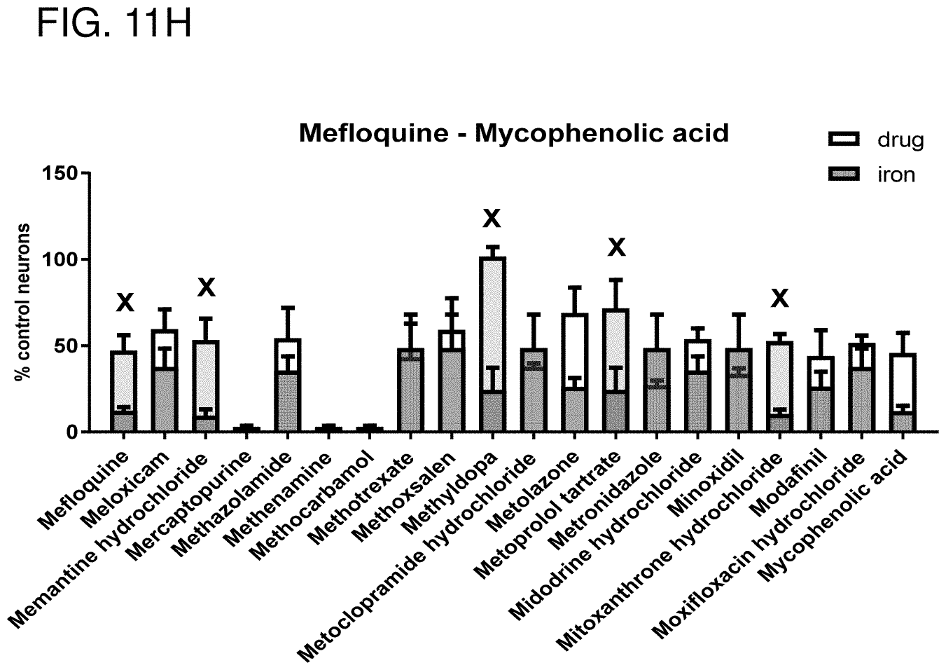

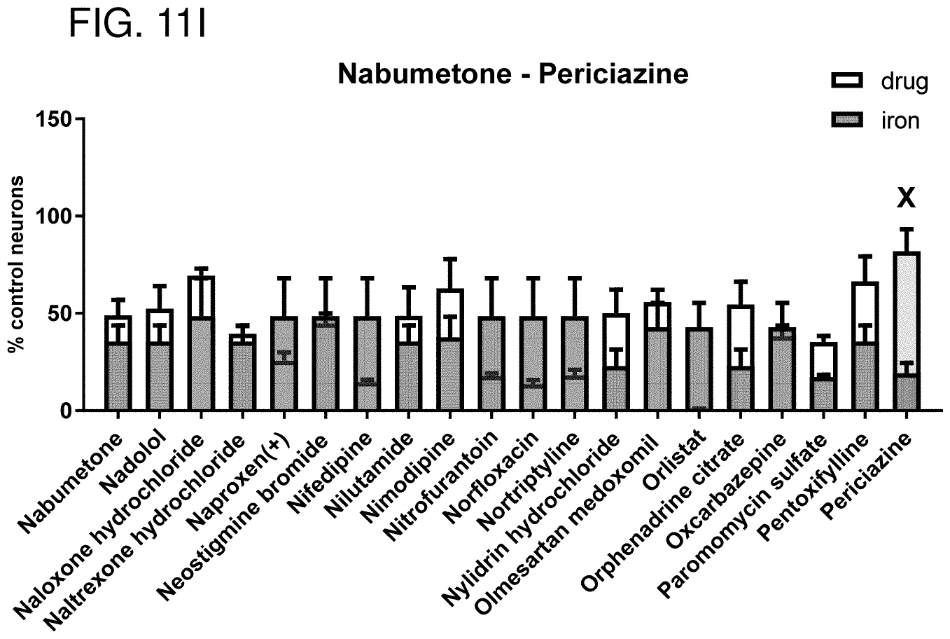

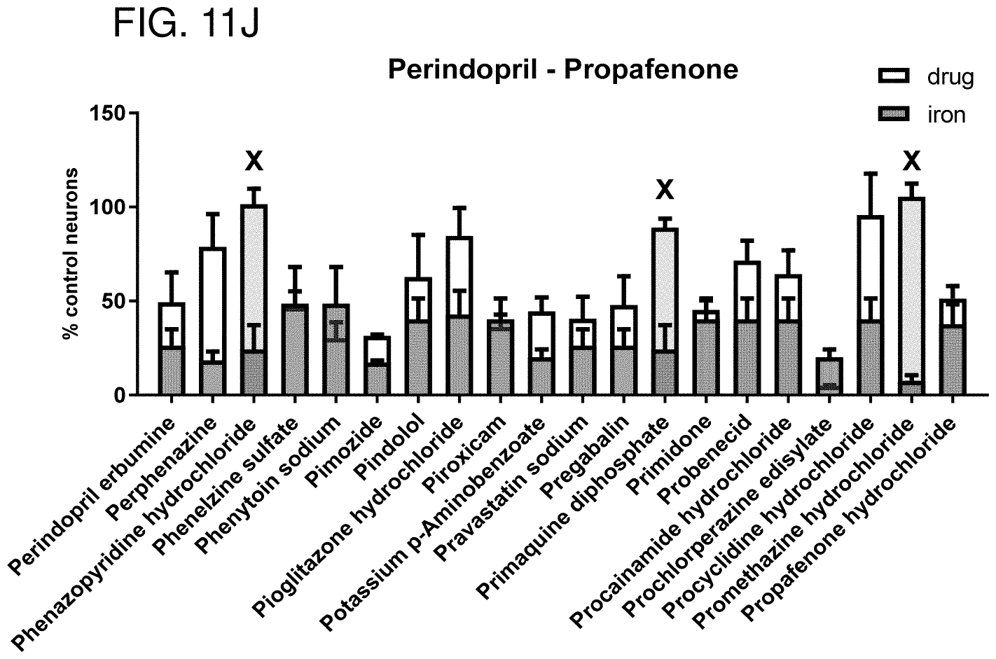

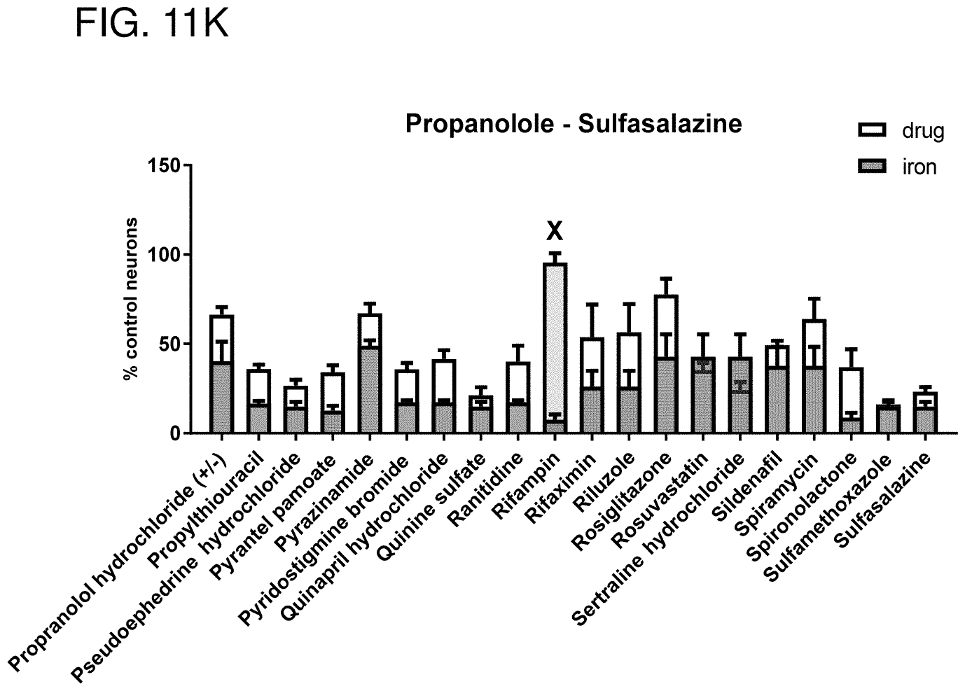

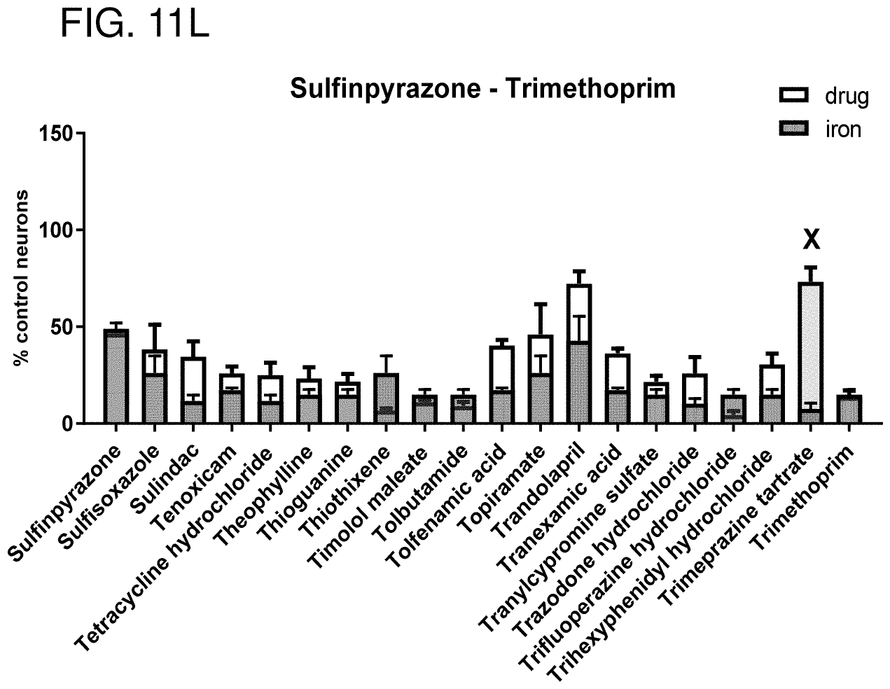

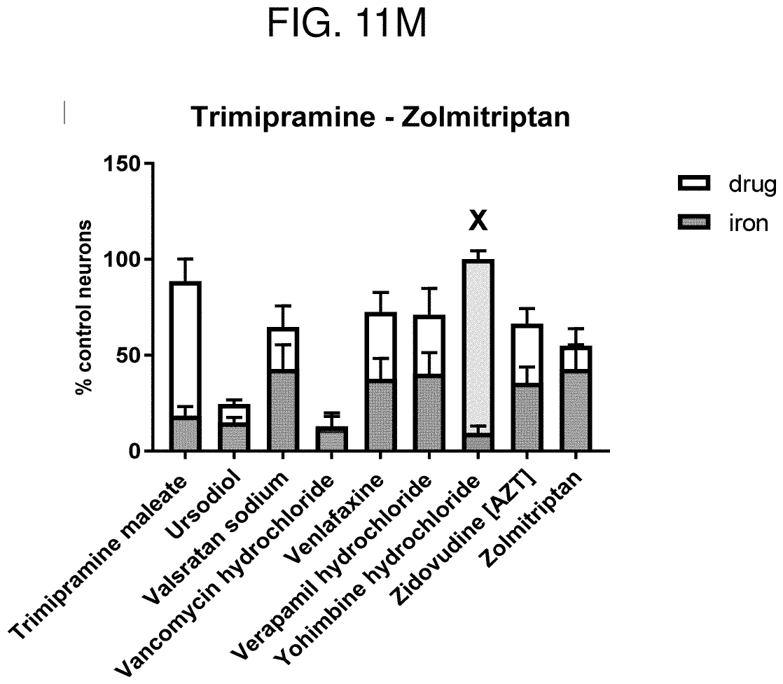

FIG. 11 (Panels A- L) shows all 249 generic compounds of the iron mediated neurotoxicity screening. The number of neurons left following exposure to each compound was normalized to the number of neurons of the respective control condition. The corresponding iron situation was also normalized to the respective control (red). Compounds which exhibit significant protection are highlighted in yellow and marked (X). Shown are the means±SEM of 1-4 experiments, performed in quadruplicates each.

Investigation of serum levels of clomipramine and its active metabolite, desmethylclomipramine (DMCL), in mice sacrificed 1 h after the last of 16 daily clomipramine injections showed mean concentrations of 751 nM and 101 nM, respectively (FIG. 8E). The corresponding mean spinal cord levels were 28 μM and 1.5 μM; a similar high brain to plasma ratio of clomipramine was reported by Marty et al. (Marty H, et al. Compared plasma and brain pharmacokinetics of clomipramine and its metabolite demethylclomipramine in two strains of mice (NMRI and CD1). Fundamental & clinical pharmacology 6, 49-57 (1992).)in mice injected with a single 8 mg/kg clomipramine IP. There was a strong correlation of serum and spinal cord levels for both clomipramine and desmethylclomipramine across mice (FIG. 8f).

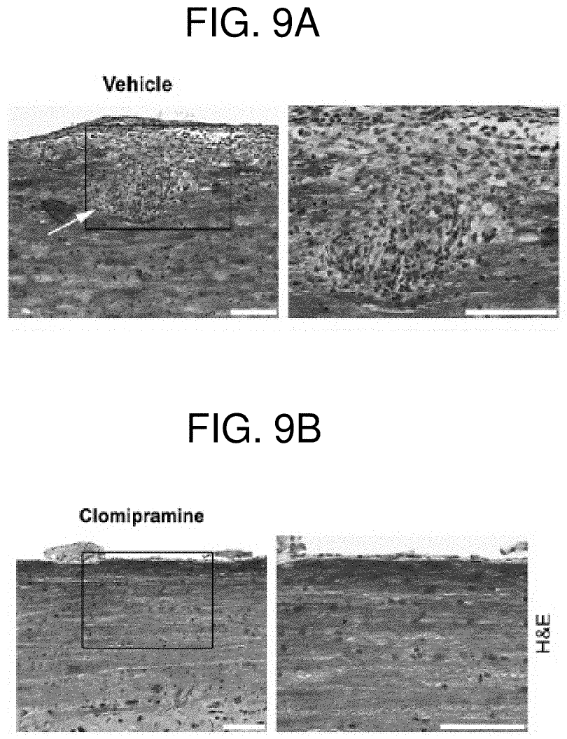

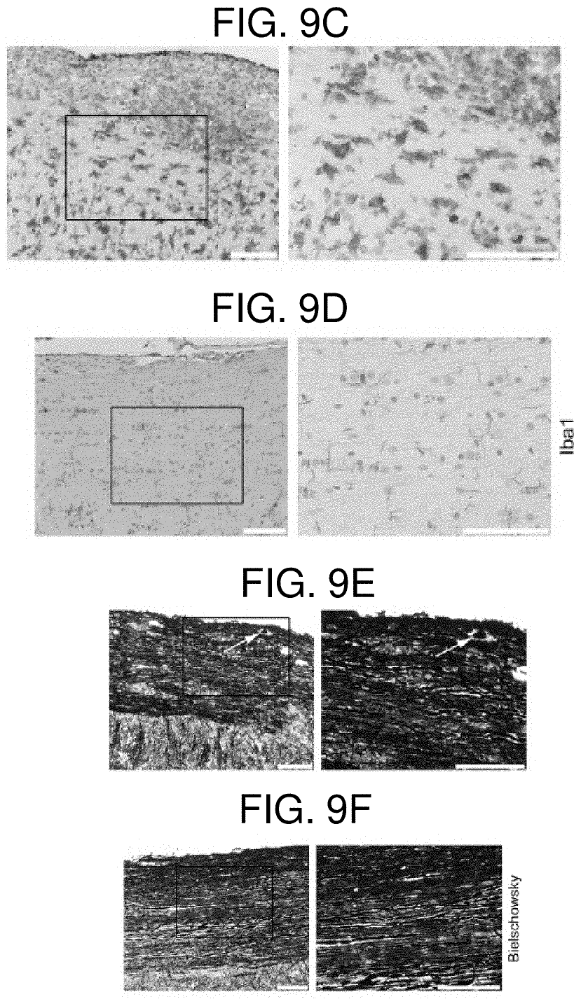

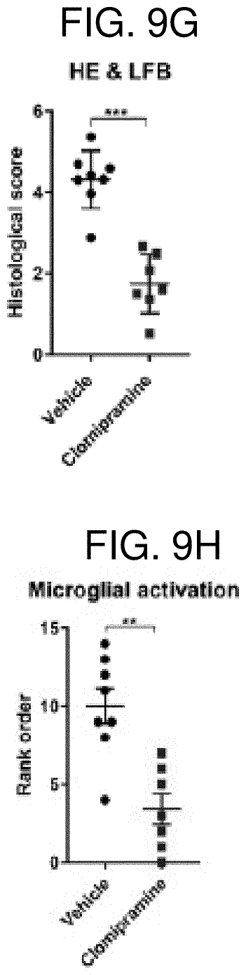

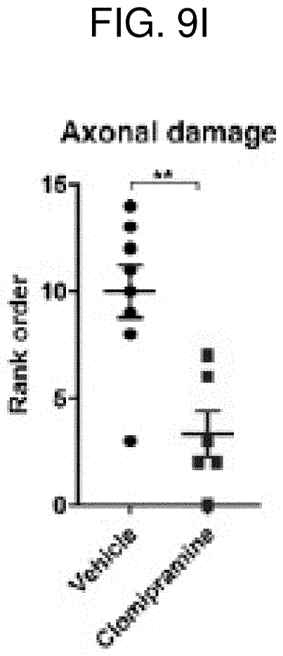

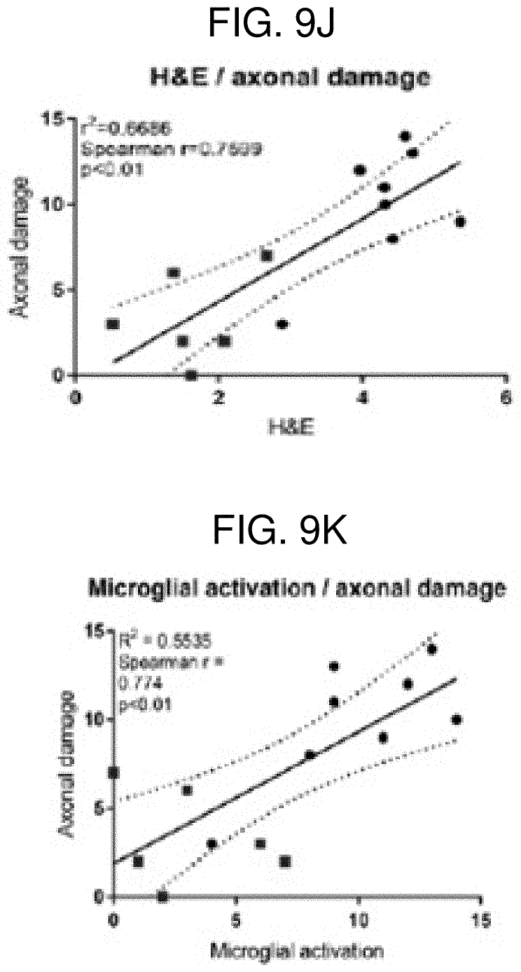

Histological analysis of the spinal cord showed profound parenchymal inflammation in vehicle treated animals with a histological score of 4.3, whereas clomipramine treated animals only had few inflammatory cells in the meninges (score 1.7; p<0.001; non-parametric two-tailed Mann-Whitney test) (FIG. 9a, b, g) that were inadequate to produce clinical manifestations as noted in FIG. 8a. Infiltration in vehicle treated animals was accompanied by massive microglial activation, whereas clomipramine treatment prevented microglial activation, as assessed by Iba1 staining (p<0.01) (FIG. 9c, d, h). Furthermore, clomipramine treated animals had significantly less axonal damage (p<0.01) (FIG. 9e, f, i). Infiltration and microglial activation correlated with axonal injury (Spearman r=0.7599, p<0.01; Spearman r=0.774, p<0.01, respectively; non-parametric two-tailed Spearman correlation with 95% confidence interval) (FIG. 9j, k).

We next set out to investigate the effect of clomipramine in chronic EAE. We first evaluated clomipramine initiated only after the first relapse when mice were in remission (day 31). In our hands, using the more sensitive 15-point EAE scoring system (rather than the conventional 5-point scale), MOG-EAE mice can be documented to undergo a second relapse after a remission period. Clomipramine did not affect the severity of the second relapse when initiated in mice at remission (FIG. 10a), likely because substantial neural injury had already occurred from a prolonged EAE course.

In another experiment, we treated MOG-immunized C57BL/6 mice from the first onset of clinical signs (day 13, FIG. 10b). Treatment with clomipramine attenuated the marked rise in clinical disability and had a significant positive effect during days 14-20 (p=0.0175; non-parametric two-tailed Mann-Whitney test). During remission, likely because the severity of disability was low, the vehicle and clomipramine treated groups did not differ. Disease was then followed by a second increase in clinical scores in vehicle-treated mice, which was prevented by clomipramine (days 42-50; p=0.0007).

Another model of chronic EAE, thought to model secondary progressive multiple sclerosis (Al-lzki S, Pryce G, Jackson S J, Giovannoni G, Baker D. Immunosuppression with FTY720 is insufficient to prevent secondary progressive neurodegeneration in experimental autoimmune encephalomyelitis. Multiple sclerosis (Houndmills, Basingstoke, England) 17, 939-948 (2011); Hampton D W, et al. An experimental model of secondary progressive multiple sclerosis that shows regional variation in gliosis, remyelination, axonal and neuronal loss. Journal of neuroimmunology 201-202, 200-211 (2008)), is immunization with spinal cord homogenate in the Biozzi ABH mouse. Clomipramine treatment was started at the onset of clinical signs where it reduced clinical severity throughout the period of treatment (p=0.0062) (FIG. 10c).

In summary, clomipramine reduced clinical severity in acute and chronic EAE in two different mouse models. FIG. 10d schematizes that the initiation of clomipramine treatment from onset of clinical signs of EAE attenuates the clinical disability observed during relapses or in chronic disease.

Discussion

Unlike relapsing-remitting multiple sclerosis, trials in progressive multiple sclerosis have largely failed so far. One important explanation is the lack of directed actions of medications against features that drive the pathophysiology of progressive multiple sclerosis, and the lack of consideration of penetration of agents into the CNS. The latter is important as the blood-brain barrier appears relatively intact in progressive compared to the relapsing-remitting form (Lassmann et al., 2012)5 , and pathogenic processes ongoing within the CNS may not be amendable to periphery-acting medications. To circumvent these challenges, we have employed bioassay screens that model aspects of progressive multiple sclerosis. Moreover, we have opted to test generic medications that have data of good access into the CNS.

One pathogenic hallmark important for the progression of multiple sclerosis is iron mediated neurotoxicity. Iron accumulates in the CNS age-dependently (Stephenson et al., 2014) and iron deposition concomitant with T cell infiltration and the expression of inducible nitric oxide synthase in microglia in the deep gray matter correlates with progression and is associated with neurodegeneration (Haider et al., 2014). The deposition of iron amplifies inflammation and exacerbates mitochondrial dysfunction through oxidative stress, eventually leading to neurodegeneration (Friese et aL, 2014). Targeting iron is thus considered a promising therapeutic approach in progressive multiple sclerosis. We investigated the potential of promising generic compounds to prevent iron mediated neurotoxicity. Out of 249 compounds screened, 35 medications which prevented against iron mediated neurotoxicity were in the drug classes of antidepressants (n=5), antibiotics (n=4), antipsychotics (n=3), antimalarials (n=2) and others. Some of the drugs had consistent outstanding neuroprotective effects, and these included antipsychotics and tricyclic antidepressants. The high number of antipsychotics and antidepressants as positive hits in the screening was striking. In addition to the rescue effect against iron mediated neurotoxicity, several drugs showed promising results in other modes of toxicity; these were desipramine, clozapine, indapamide and labetalol which were active against damage to the mitochondrial respiratory chain. Data were corroborated by the investigation of antioxidative potential and the influence on splenocyte proliferation. Clomipramine showed outstanding effects in several in vitro settings such as against iron mediated neurotoxicity, hydroxyl scavenging capacity, and inhibition of T- and B-cell proliferation; in mice, clomipramine suppressed occurrence of disease in EAE completely, concomitant with reduced transcripts of chemotactic and inflammatory cytokines in the spinal cord, reduced inflammation, microglial activation and preservation of axons. Moreover, clomipramine ameliorated clinical signs in chronic EAE in two different EAE models, C57BL/6 and Biozzi ABH mice.

The work presented here constitutes a systematic approach to identify generic compounds that could be useful for the treatment of progressive multiple sclerosis. First, we focused on ameliorating major hallmarks of progressive multiple sclerosis such as iron-mediated neurotoxicity, oxidative stress and immune cell proliferation. Second, we chose generic drugs which are available as oral formulations. The drugs have a well-known safety-profile, as there exists long-lasting experience in research and clinical use.

Some of the compounds that prevented iron-mediated neurotoxicity in our screen have been described previously to have neuroprotective properties and will be highlighted here, as they may be of interest not only to progressive multiple sclerosis but also other CNS disorders with neurodegenerative features. Strong neuroprotective effects were induced by tricyclic antidepressants. The antidepressant desipramine has been used in a Huntington's disease model where it inhibited glutamate-induced mitochondrial permeability at the concentration of 2 μM and led to reduced apoptosis of primary murine neurons (Lauterbach EC. Neuroprotective effects of psychotropic drugs in Huntington's disease. International journal of molecular sciences 14, 22558-22603 (2013); Tang T S, et al. Disturbed Ca2+ signaling and apoptosis of medium spiny neurons in Huntington's disease. Proceedings of the National Academy of Sciences of the United States of America 102, 2602-2607 (2005)). Furthermore, desipramine induces the anti-oxidative enzyme heme-oxygenase 1 in Mes23.5 dopaminergic cells and increases Nrf2 accumulation in the nucleus, thus preventing neuronal cell death mediated by rotenone and 6-hydroxydopamine (Lin H Y, et al. Desipramine protects neuronal cell death and induces heme oxygenase-1 expression in Mes23.5 dopaminergic neurons. PloS one 7, e50138 (2012).

Besides desipramine, other tricyclic antidepressants had strong effects against splenocyte proliferation. Imipramine, which showed good neuroprotective properties, enhances PEP-1-catalase in astrocytes, leading to neuroprotection in the hippocampal CA1 region in an ischemia model (Kim DW, et al. Imipramine enhances neuroprotective effect of PEP-1-Catalase against ischemic neuronal damage. BMB reports 44, 647-652 (2011).) Additionally, it prevents apoptosis of neural stem cells by lipopolysaccharide, mediated by the brain derived neurotrophic factor (BDNF) and mitogen-activated protein kinase (MAPK) pathway (Peng CH, et al. Neuroprotection by Imipramine against lipopolysaccharide-induced apoptosis in hippocampus-derived neural stem cells mediated by activation of BDNF and the MAPK pathway. European neuropsychopharmacology: the journal of the European College of Neuropsychopharmacology 18, 128-140 (2008)). Another novel compound recently developed, quinpramine, which is a fusion of imipramine and the anti-malarial quinacrine, decreased the number of inflammatory CNS lesions, antigen-specific T-cell proliferation and pro-inflammatory cytokines in EAE (Singh MP, et al. Quinpramine is a novel compound effective in ameliorating brain autoimmune disease. Exp Neurol 215, 397-400 (2009).).

Due to structural similarities between clomipramine, imipramine and trimipramine it may be speculated that these compounds may be relevant for trials in progressive multiple sclerosis. Furthermore, we showed previously that doxepin reduces microglial activation to 46% without inducing toxicity; clomipramine, however, did not have microglia inhibitory activity 14. In the synopsis of effects contributing to progressive multiple sclerosis, tricyclic antidepressants are interesting for further development and might even be suitable as combination therapy with other compounds targeting features of progressive multiple sclerosis.

Some antipsychotics also displayed strong protection against iron and oxidative stress. Clozapine has been described to reduce microglial activation through inhibition of phagocytic oxidase (PHOX)-generated reactive oxygen species production, mediating neuroprotection (Hu X, et al. Clozapine protects dopaminergic neurons from inflammation-induced damage by inhibiting microglial overactivation. Journal of neuroimmune pharmacology: the official journal of the Society on Neurolmmune Pharmacology 7, 187-201 (2012)). The strong anti-oxidative properties of clozapine in the HORAC assay support these results. Due to the side effect profile with enhanced risk of agranulocytosis, we refrained from usage in EAE; nevertheless, in multiple sclerosis patients with psychiatric comorbidities and eligible for antipsychotic treatment, it may be reasonable to use clozapine.

With regards to liothyronine, atenolol or carvedilol that prevented iron-mediated neurotoxicity beyond levels of controls, these do not penetrate the CNS (probability of 68% for all three, drugbank.ca) as well as clomipramine (97.9% chance for entering the CNS according to drugbank.ca). Thus, we did not explore their utility in EAE.

Mitoxantrone is used in some countries as a treatment for progressive multiple sclerosis, but has so far not yet been described as being neuroprotective. Although the blood-brain-barrier permeability probability is poor (0.7979), it may be postulated that the effect in progressive multiple sclerosis, in addition to its toxic effects on T-lymphocytes, is induced by its capacity to limit iron-mediated neurotoxicity. Indapamide exhibited strong neuroprotective effects against iron toxicity in culture, which has not yet been described previously. More interestingly, indapamide also overcomes mitochondrial damage. As indapamide has no effect on T-lymphocyte proliferation, the drug may not overcome acute-EAE, but may be interesting in longer term multiple sclerosis models such as the Biozzi ABH mouse model, which shows immune cell-independent neurodegeneration 35 and a chronic disease course 22.

As noted in FIG. 17, indapamide alleviates oxidative stress observed in the spinal cord following demyelination induced by lysolecithin in this area. Specifically, the lysolecithin injury to the spinal cord particularly in aging 8-10 month old mice (thought to reflect middle age in humans, an age commonly associated with progression of disability in primary progressive and secondary progressive MS) led to the activation of NADPH oxidase, whose activation has also been noted in MS particularly in progressive M S (Haider L, Fischer M T, Frischer J M, Bauer J, Hoftberger R, Botond G, Esterbauer H, Binder C J, Witztum J L, Lassmann H, Oxidative damage in multiple sclerosis lesions., Brain 134:1914-1924, 2011). Treatment with indapamide reduces oxidative stress-mediated lipid oxidation as indicated by measurement of malondialdehyde expression within the demyelinated lesion, and resulted in reduced myelin and axonal loss caused by the lysolecithin (FIG. 17).

We opted to test clomipramine in the acute-EAE model due to its strong effects on immune cells, its antioxidative properties and its prevention against iron mediated neurotoxicity. Clomipramine is a tricyclic antidepressant which is used to treat depression, obsessive compulsive disorder and panic disorders, usually in a dosage of 100-150 mg/d, sometimes up to 300 mg/d. It inhibits serotonin and norepinephrine uptake. Clomipramine reduces the seizure threshold and overdose can lead to cardiac dysrhythmias, hypotension and coma (drugbank.ca). Usually, clomipramine is well tolerated, but side effects include amongst others increase in weight, sexual dysfunctions, sedation, hypotension and anticholinergic effects such as dry mouth, sweating, obstipation, blurred vision and micturition disorder (according to the manufacturer leaflet). Clomipramine crosses readily into the CNS with a probability to cross the blood brain barrier of 0.979 according to predicted ADMET (absorption, distribution, metabolism, excretion, toxicity) features (drugbank.ca). Clomipramine reduces the production of nitric oxide and TNF-α in microglia and astrocytes (Hwang et al., 2008); the authors reported neuroprotective properties in a co-culture model of neuroblastoma cells and microglia. Clomipramine increases the uptake of cortisol in primary rat neurons (Pariante et al., 2003) and promotes the release of glial cell line-derived neurotrophic factor in glioblastoma cells, suggesting a protective effect on neurons (Hisaoka et al., 2001). The drug has been also studied in experimental autoimmune neuritis, where it decreases the number of IFN-γ secreting Th1 cells and ameliorated the clinical course (Zhu et al., 1998).

Clomipramine has been used previously in mice in different dosages to study conditions such as anti-nociception (0.5 mg/kg) (Schreiber et aL, 2015), Chagas disease (7.5 mg/kg) (Garcia et al., 2016) and neurotransmitter and histone deacetylase expression (50 mg/kg) (Ookubo et al., 2013). In humans taking clomipramine as an anti-depressant, mean serum levels after a mean daily intake of 127±91 mg/d have been reported to be 122 ng/ml (387 nM, considering a molecular weight of 314.9) (Rodriguez de la Torre et al., 2001). Of note, clomipramine levels after oral intake in humans have a wide range, leading to plasma concentrations of more than 600 nM in some individuals (Thoren et al., 1980), which is in the range of neuroprotection against iron in our in vitro experiments. The injection of 20 mg/kg IP in CD1 mice leads to peak plasma concentrations of 438 ng/ml (1.4 μM) with a half-life of 165 min (Marty et al., 1992), and in our experiments animals (sacrificed 1 h after the last injection) had mean serum clomipramine concentrations of 236.5 ngeml (751 nM). These plasma levels are close to the ones measured in humans (average of 387 nM, and up to 600 nM (Thoren et al., 1980)), especially keeping in mind that plasma levels drop faster in mice due to the relatively bigger liver:body mass and that the half-life of clomipramine in humans is between 17.7 and 84 hours (Balant-Gorgia et al., 1991) compared to about 2.5 h in mice. We found that clomipramine levels in the spinal cord of the EAE-afflicted mice averaged 28 μM; levels achieved in the brains of humans are not known. Thus, the dosage of 25 mg/kg clomipramine tested in our EAE study reflects standard dose used in humans in that both attain similar plasma levels.

In summary, we discovered several generic compounds in this systematic screening approach that exhibit neuroprotective properties against iron-mediated neurotoxicity. Additionally, some of those compounds prevent mitochondrial damage to neurons, inhibit immune cell proliferation and show anti-oxidative capacities. Tricyclic antidepressants, antipsychotics and indapamide may be useful for further development in progressive multiple sclerosis due to their manifold properties. Clomipramine showed particular promise due to its capacity to reduce iron-mediated neurotoxicity and T- and B-cell proliferation, its anti-oxidative effect, and its complete suppression of disease in acute-EAE and positive effects in chronic EAE.

### Example 2

### Indapamide Reduces Myelin and Axon Loss in an MS Model

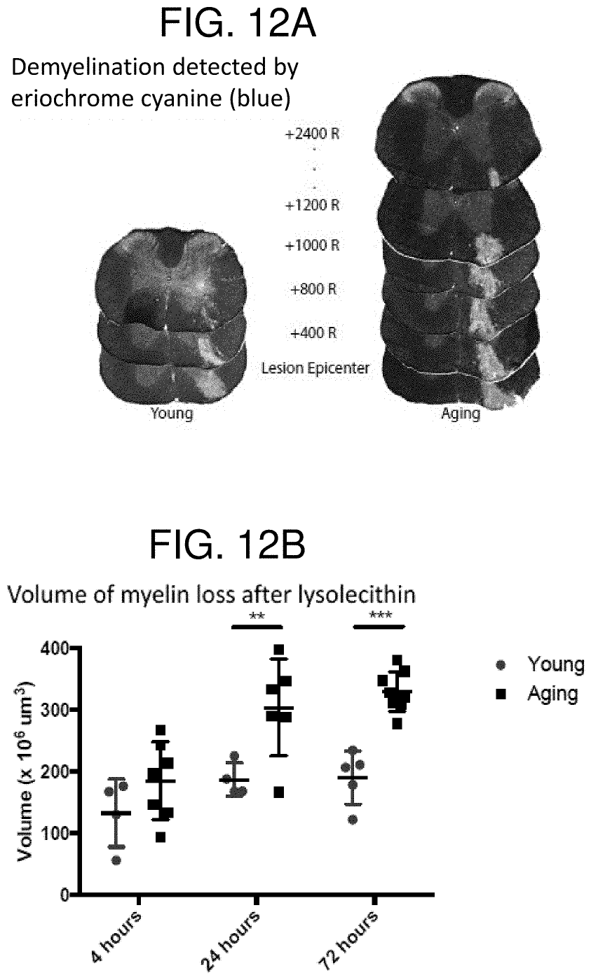

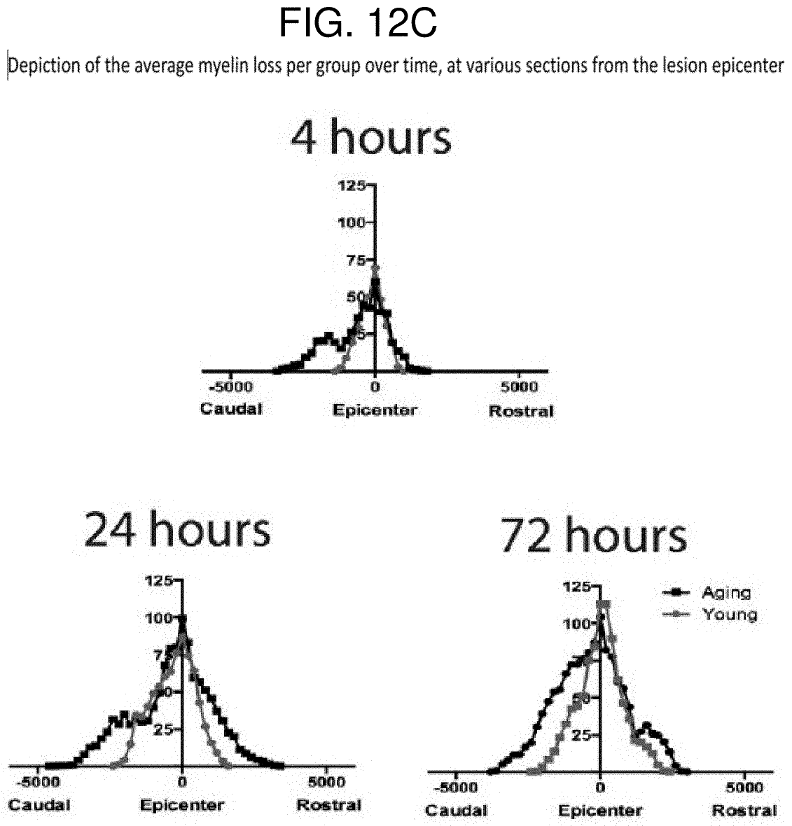

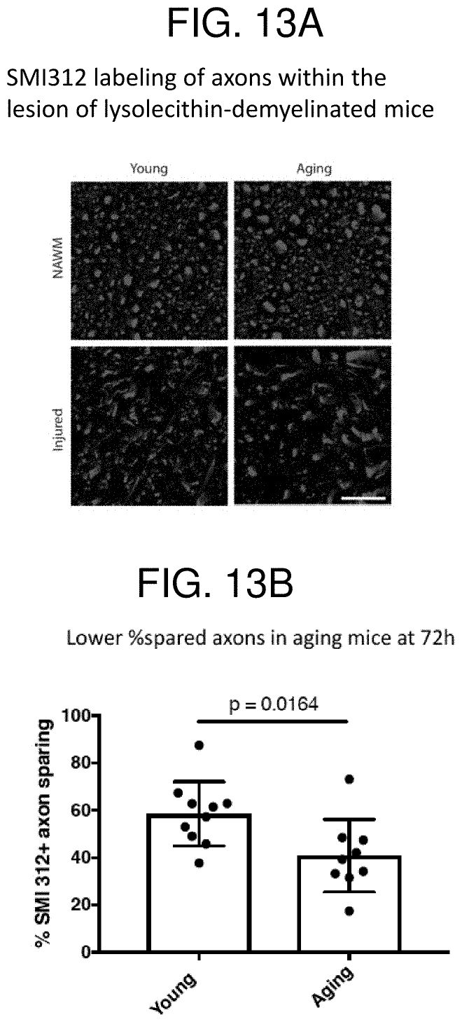

Active demyelinating lesions can be found in MS specimens of all ages sampled, including late in life. Indeed, age has been identified to be a factor in the dreaded conversion from relapsing-remitting into secondary progressive MS. Contributing causes for aging-associated worsening in MS that drives progression include the steady loss of axons with longevity of disease, or the deficient repair of myelin in older compared to younger patients. We tested the hypothesis that the same demyelinating injury is more devastating to axons and myelin as the individual ages. Indeed, using the lysolecithin model of demyelination in the spinal cord white matter of mice (as performed in Keough et al., Experimental demyelination and remyelination of murine spinal cord by focal injection of lysolecithin, J Visualized Experiments March 26;(97). doi: 10.3791/52679), we found that an identical lysolecithin insult to the spinal cord produces by 24h to 72h a larger volume of demyelination and axonal loss in 8-10 months old mice compared to young 6 weeks old animals (FIG. 12,13).

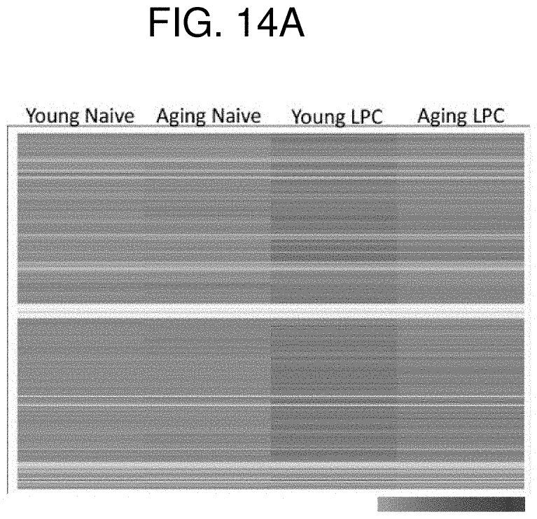

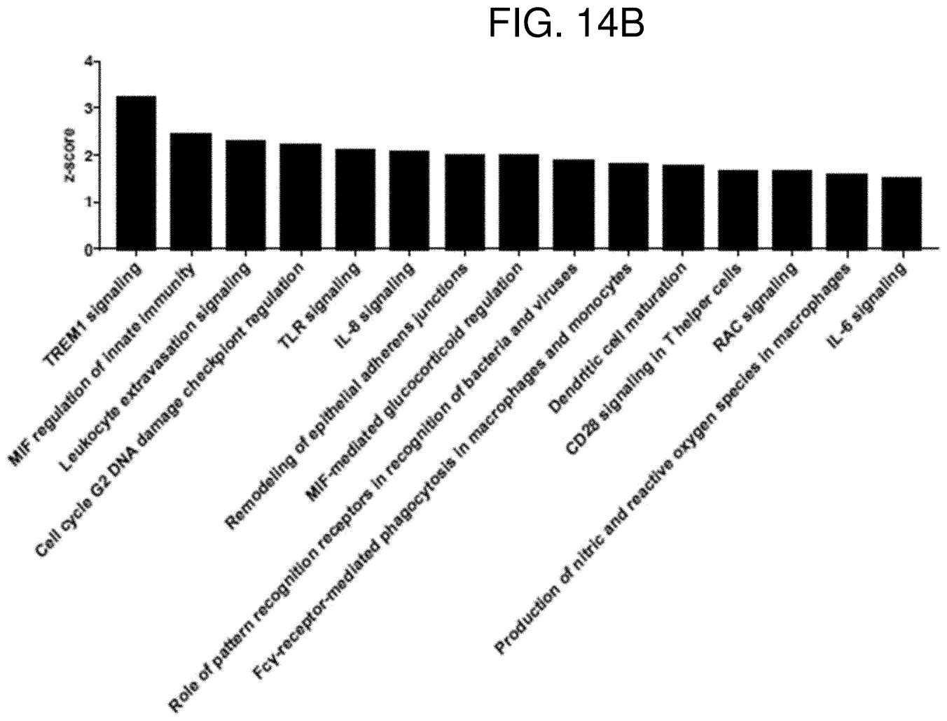

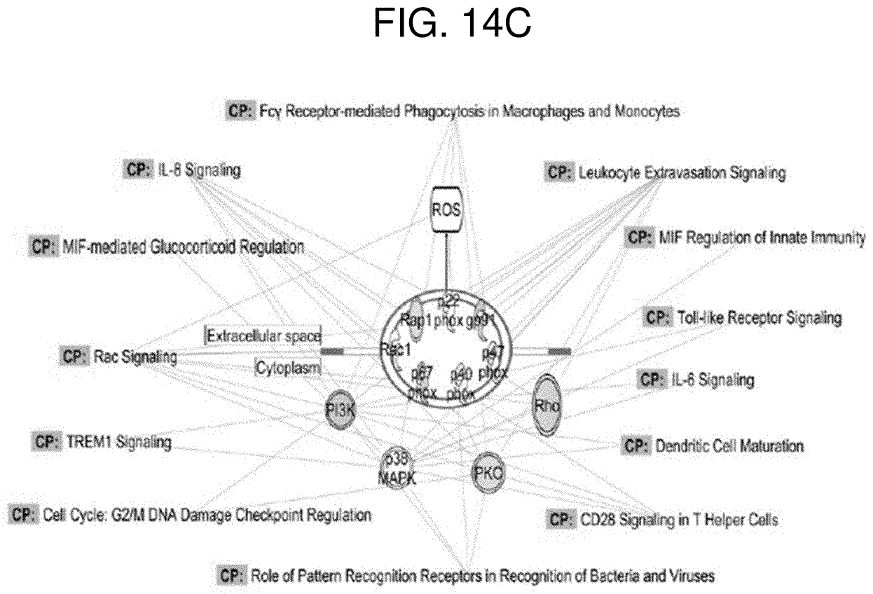

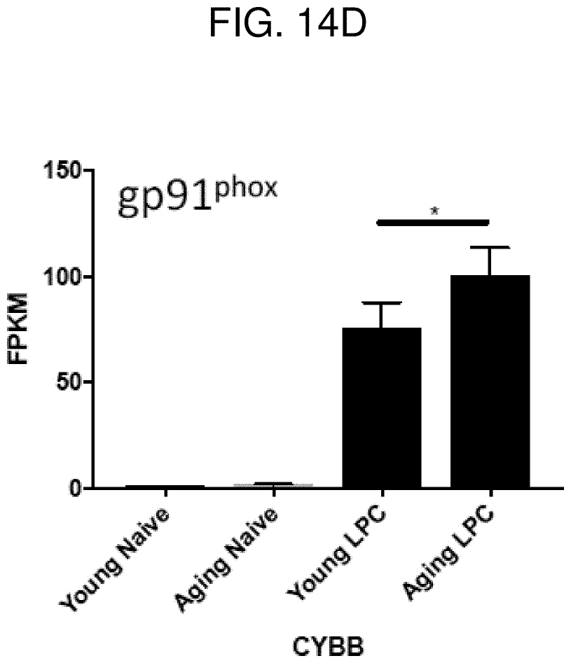

FIG. 14 shows RNAseq data of 3day laser-microdissected lesions that homed onto NADPH oxidase. a) Heat map (3 samples/group, where each sample is a pool of 5 mice) after lysolecithin (LPC) lesion in young and aging mice. b) Upregulation of canonical immune-associated pathways in aging vs young mice that converge, through Ingenuity Pathway Analysis, into NADPH oxidase 2 subunits. d) The RNAseq levels of the catalytic subunit of NADPH oxidase 2, gp91phox (also called CYBB) are selected for display. *p<0.05.

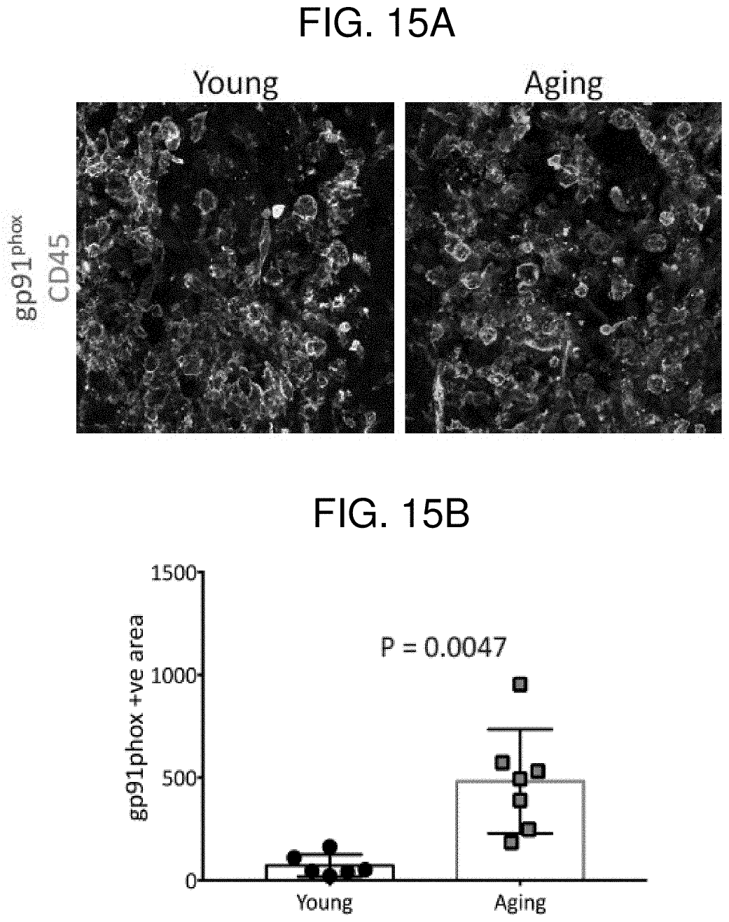

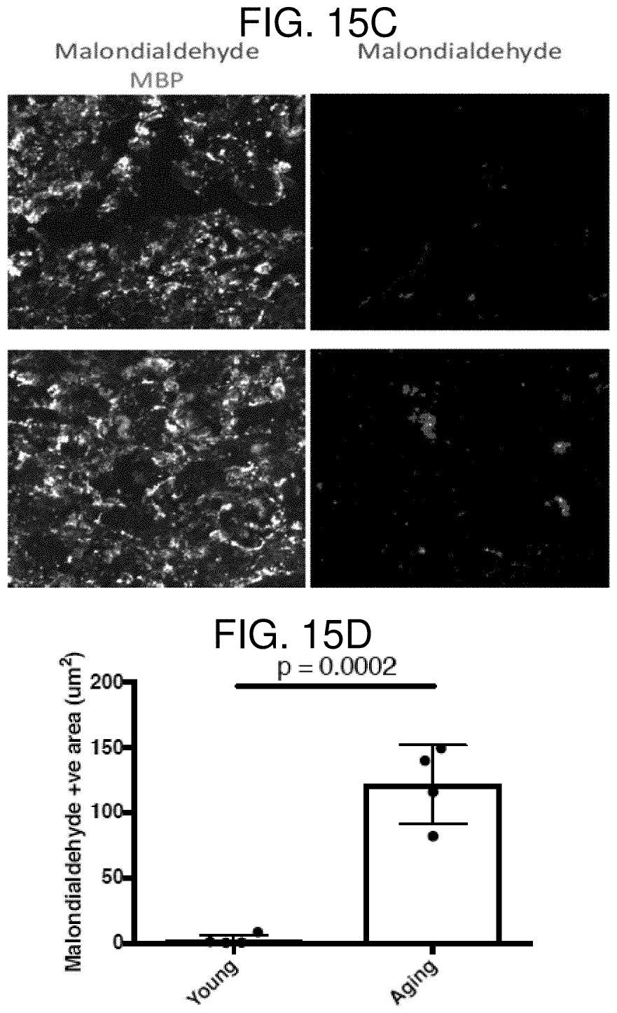

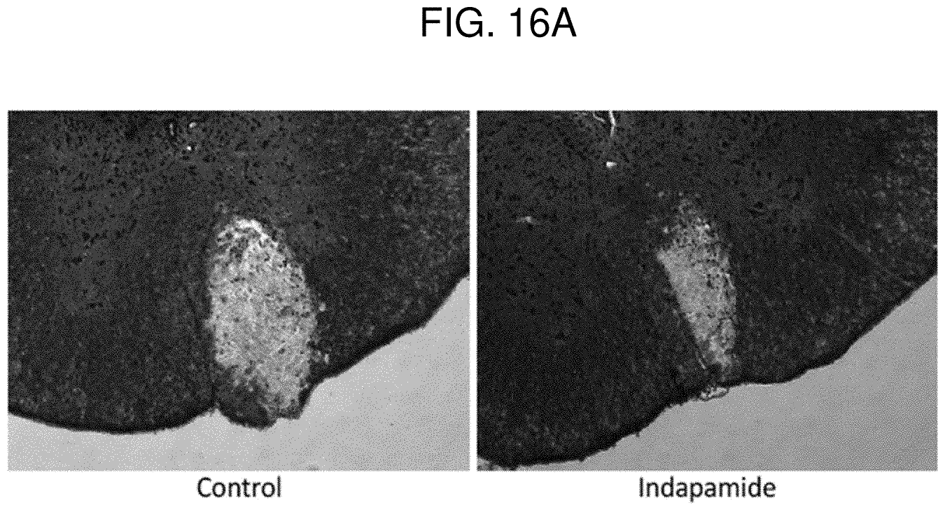

FIG. 15 shows higher expression of gp91^{phox} (an NADPH oxidase subunit) and malondialdehyde in aging lesions. a,b) The catalytic subunit of NOX2, gp91phox, is readily found within CD45+ cells in aging but not young demyelinated lesions (d3). (c,d) Similarly, malondialdehyde as a marker of oxidative damage is in aging lesion associated with MBP+ myelin breakdown.

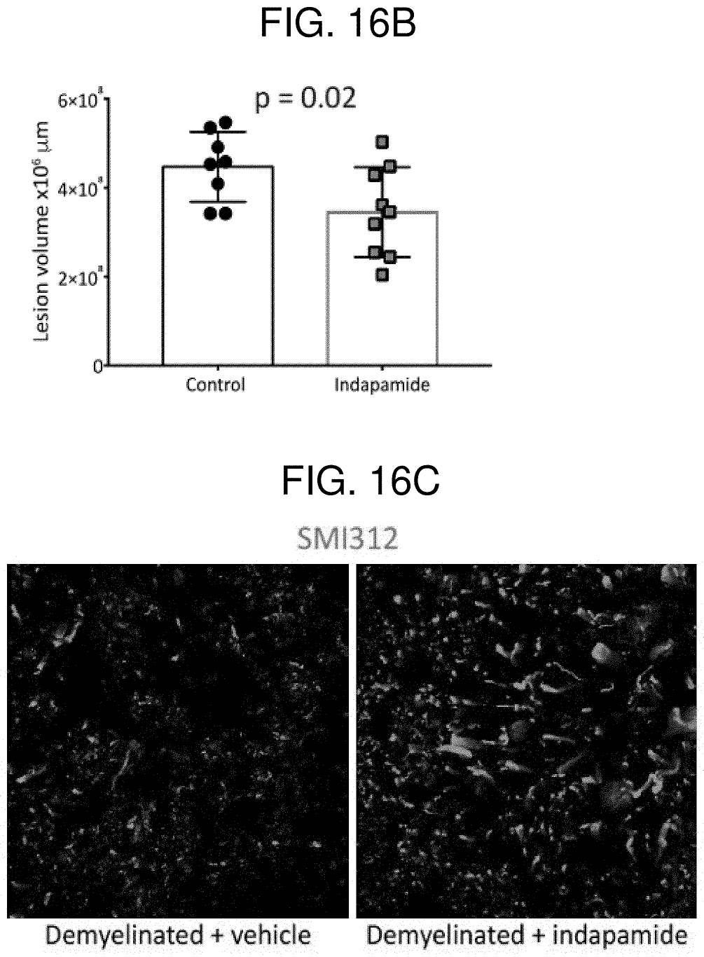

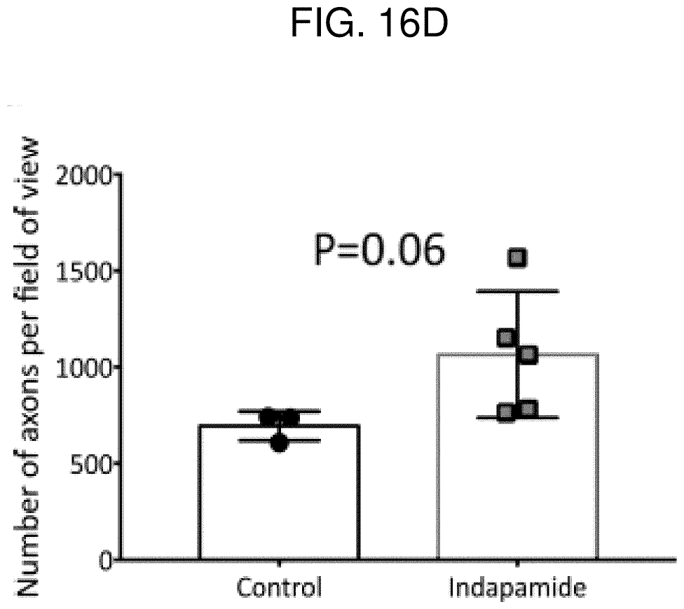

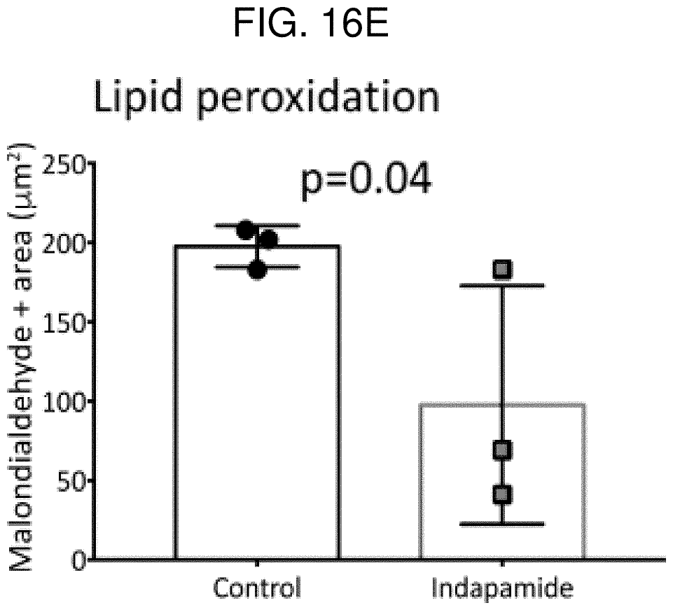

Since we found oxidative stress more prevalent within the lysolecithin lesion of the aging mice, we tested indapamide, a well-tolerated angiotensin converting enzyme inhibitor used as an anti-hypertensive, as it has strong anti-oxidant properties as described in the appended manuscript. Also, indapamide limits the neurotoxicity of the MS-relevant insult iron in culture. We thus treated aging 8-10 months old mice with intraperitoneal indapamide (20 mg/kg) immediately after lysolecithin demyelination, and once per day at 20 mg/kg for the next 2 days. Spinal cord tissues were taken for histology. We found that indapamide-treated mice have a smaller volume of demyelination, less axonal loss, and reduced lesional malondialdehyde (a marker of oxidant-mediated injury) level (FIG. 16) than their vehicle-administered controls. These results suggest the potential of indapamide as a medication for progressive MS.

List of Abbreviations

BDNF: Brain-derived neurotrophic factor

DMSO: Dimethyl sulfoxide

EAE: Experimental autoimmune encephalomyelitis

FBS: Fetal bovine serum

GAEs: Gallic acid equivalents

HORAC: Hydroxyl radical antioxidant capacity

INN: International nonproprietary name

IP: Intraperitoneal

JAN: Japanese Accepted Name

MAP-2: Microtubule-associated protein-2

MAPK: Mitogen-activated protein kinases

MEM: Minimal essential medium

PFA: Paraformaldehyde

PI: Propidium iodide

PPMS: Primary-progressive multiple sclerosis

RRMS: Relapsing-remitting multiple sclerosis

USAN: United States Adopted Names

USP: United States Pharmacopeia

### REFERENCE LIST

Al-lzki S, Pryce G, Jackson S J, Giovannoni G, Baker D. Immunosuppression with FTY720 is insufficient to prevent secondary progressive neurodegeneration in experimental autoimmune encephalomyelitis. Multiple sclerosis (Houndmills, Basingstoke, England) 2011; 17(8): 939-48.

Balant-Gorgia A E, Gex-Fabry M, Balant L P. Clinical pharmacokinetics of clomipramine. Clinical pharmacokinetics 1991; 20(6): 447-62.

Besson A, Yong V W. Involvement of p21(Waf1/Cip1) in protein kinase C alpha-induced cell cycle progression. Molecular and cellular biology 2000; 20(13): 4580-90.

Campbell G R, Worrall J T, Mahad D J. The central role of mitochondria in axonal degeneration in multiple sclerosis. Multiple sclerosis (Houndmills, Basingstoke, England) 2014; 20(14): 1806-13.

Campbell G R, Ziabreva I, Reeve A K, Krishnan K J, Reynolds R, Howell O, et al. Mitochondrial DNA deletions and neurodegeneration in multiple sclerosis. Ann Neurol 2011; 69(3): 481-92.

Ciz M C, H.; Denev, P.; Kratchanova, M.; Slavov, A.; Lojek, A. Different methods for control and comparison of the antioxidant properties of vegetables. FOOD CONTROL 2010; 21: 518-23.

Di Prisco S, Merega E, Lanfranco M, Casazza S, Uccelli A, Pittaluga A. Acute desipramine restores presynaptic cortical defects in murine experimental autoimmune encephalomyelitis by suppressing central CCL5 overproduction. British journal of pharmacology 2014; 171(9): 2457-67.

Fischer M T, Sharma R, Lim J L, Haider L, Frischer J M, Drexhage J, et al. NADPH oxidase expression in active multiple sclerosis lesions in relation to oxidative tissue damage and mitochondrial injury. Brain 2012; 135(Pt 3): 886-99.

Fraussen J, de Bock L, Somers V. B cells and antibodies in progressive multiple sclerosis: Contribution to neurodegeneration and progression. Autoimmunity reviews 2016; 15(9): 896-9.

Friese M A, Schattling B, Fugger L. Mechanisms of neurodegeneration and axonal dysfunction in multiple sclerosis. Nat Rev Neurol 2014; 10(4): 225-38.

Frischer J M, Weigand S D, Guo Y, Kale N, Parisi J E, Pirko I, et aL Clinical and pathological insights into the dynamic nature of the white matter multiple sclerosis plaque. Ann Neurol 2015; 78(5): 710-21.

Garcia M C, Ponce N E, Sanmarco L M, Manzo R H, Jimenez-Kairuz A F, Aoki M P. Clomipramine and Benznidazole Act Synergistically and Ameliorate the Outcome of Experimental Chagas Disease. Antimicrobial agents and chemotherapy 2016; 60(6): 3700-8.

Giuliani F, Fu S A, Metz L M, Yong V W. Effective combination of minocycline and interferon-beta in a model of multiple sclerosis. Journal of neuroimmunology 2005; 165(1-2): 83-91.

Haider L, Simeonidou C, Steinberger G, Hametner S, Grigoriadis N, Deretzi G, et al. Multiple sclerosis deep grey matter: the relation between demyelination, neurodegeneration, inflammation and iron. J Neurol Neurosurg Psychiatry 2014; 85(12): 1386-95.

Hametner S, Wimmer I, Haider L, Pfeifenbring S, Bruck W, Lassmann H. Iron and neurodegeneration in the multiple sclerosis brain. Ann Neurol 2013; 74(6): 848-Hampton D W, Serio A, Pryce G, Al-lzki S, Franklin R J M, Giovannoni G, et aL Neurodegeneration progresses despite complete elimination of clinical relapses in a mouse model of multiple sclerosis. Acta Neuropathologica Communications 2013; 1(1): 84.

Hisaoka K, Nishida A, Koda T, Miyata M, Zensho H, Morinobu S, et al. Antidepressant drug treatments induce glial cell line-derived neurotrophic factor (GDNF) synthesis and release in rat C6 glioblastoma cells. Journal of neurochemistry 2001; 79(1): 25-34.

Hu X, Zhou H, Zhang D, Yang S, Qian L, Wu H M, et al. Clozapine protects dopaminergic neurons from inflammation-induced damage by inhibiting microglial overactivation. Journal of neuroimmune pharmacology : the official journal of the Society on Neurolmmune Pharmacology 2012; 7(1): 187-201.

Hwang J, Zheng L T, Ock J, Lee M G, Kim S H, Lee H W, et al. Inhibition of glial inflammatory activation and neurotoxicity by tricyclic antidepressants. Neuropharmacology 2008; 55(5): 826-34.

Kappos L, Bar-Or, A.; Cree, B.; Fox, R.; Giovannoni, G.; Gold, R.; Vermersch, P.; Arnould, S.; Sidorenko, T.; Wolf, C.; Wallstroem, E.; Dahlke F. Efficacy and safety of siponimod in secondary progressive multiple sclerosis—Results of the placebo controlled, double-blind, Phase III EXPAND study. ECTRIMS Online Library 2016.

Keough MB, Rogers JA, Zhang P, Jensen SK, Stephenson EL, Chen T, et al. An inhibitor of chondroitin sulfate proteoglycan synthesis promotes central nervous system remyelination. Nature communications 2016; 7: 11312.

Kim D W, Kim D S, Kim M J, Kwon S W, Ahn E H, Jeong H J, et al. Imipramine enhances neuroprotective effect of PEP-1-Catalase against ischemic neuronal damage. BMB reports 2011; 44(10): 647-52.

Koch M W, Glazenborg A, Uyttenboogaart M, Mostert J, De Keyser J. Pharmacologic treatment of depression in multiple sclerosis. The Cochrane database of systematic reviews 2011(2): Cd007295.

Koch M W, Zabad R, Giuliani F, Hader W, Jr., Lewkonia R, Metz L, et al. Hydroxychloroquine reduces microglial activity and attenuates experimental autoimmune encephalomyelitis. Journal of the neurological sciences 2015; 358(1-2): 131-7.

Komori M, Blake A, Greenwood M, Lin Y C, Kosa P, Ghazali D, et al. Cerebrospinal fluid markers reveal intrathecal inflammation in progressive multiple sclerosis. Ann Neurol 2015; 78(1): 3-20.

L. M. Metz L M L, D.; Traboulsee, A.; Duquette, P.; Yong, V. W.; Eliasziw, M.; Cerchiaro, G.; Greenfield, J.; Riddehough, A.; Yeung, M.; Kremenchutzky, M.; Vorobeychik, G.; Freedman, M. S.; Bhan, V.; Blevins, G.; Marriott, J. J.; Grand'Maison, F.; Lee, L.; Thibault, M.; Hill, M. Minocycline reduces the relative risk of multiple sclerosis in people experiencing their first clinical demyelinating event by 44.6%: results of a phase III double-blind placebo controlled Canadian multicentre clinical trial. ECTRIMS Online Library 2015.

Lassmann H, van Horssen J, Mahad D. Progressive multiple sclerosis: pathology and pathogenesis. Nat Rev Neurol 2012; 8(11): 647-56.

Lau L W, Keough M B, Haylock-Jacobs S, Cua R, Doring A, Sloka S, et al. Chondroitin sulfate proteoglycans in demyelinated lesions impair remyelination. Ann Neurol 2012; 72(3): 419-32.

Lauterbach E C. Neuroprotective effects of psychotropic drugs in Huntington's disease. International journal of molecular sciences 2013; 14(11): 22558-603.

Li R, Rezk A, Miyazaki Y, Hilgenberg E, Touil H, Shen P, et al. Proinflammatory GM-CSF-producing B cells in multiple sclerosis and B cell depletion therapy. Science translational medicine 2015; 7(310): 310ra166.

Lin H Y, Yeh W L, Huang B R, Lin C, Lai C H, Lin H, et al. Desipramine protects neuronal cell death and induces heme oxygenase-1 expression in Mes23.5 dopaminergic neurons. PloS one 2012; 7(11): e50138.

Lublin F, Miller D H, Freedman M S, Cree B A, Wolinsky J S, Weiner H, et al. Oral fingolimod in primary progressive multiple sclerosis (INFORMS): a phase 3, randomised, double-blind, placebo-controlled trial. Lancet (London, England) 2016.

Marty H, Varoquaux O, Fialip J, Cordonnier P, Duroux E, Bulach C, et al. Compared plasma and brain pharmacokinetics of clomipramine and its metabolite demethylclomipramine in two strains of mice (NMRI and CD1). Fundamental & clinical pharmacology 1992; 6(2): 49-57.

Montalban X H, B.; Rammohan, K.; Giovannoni, G.; de Seze, J.; Bar-Or, A.; Arnold, D. L.; Sauter, A.; Kakarieka, A.; Masterman, D.; Chin, P.; Garren, H.; Wolinsky, J.; on behalf of the ORATORIO Clinical Investigators. Efficacy and safety of ocrelizumab in primary progressive multiple sclerosis—results of the placebo-controlled, double-blind, Phase III ORATORIO study. ECTRIMS Online Library 2015.

Ookubo M, Kanai H, Aoki H, Yamada N. Antidepressants and mood stabilizers effects on histone deacetylase expression in C57BL/6 mice: Brain region specific changes. Journal of psychiatric research 2013; 47(9): 1204-14.

Ou B, Hampsch-Woodill M, Flanagan J, Deemer E K, Prior R L, Huang D. Novel fluorometric assay for hydroxyl radical prevention capacity using fluorescein as the probe. Journal of agricultural and food chemistry 2002; 50(10): 2772-7.

Pariante C M, Hye A, Williamson R, Makoff A, Lovestone S, Kerwin R W. The antidepressant clomipramine regulates cortisol intracellular concentrations and glucocorticoid receptor expression in fibroblasts and rat primary neurones. Neuropsychopharmacology: official publication of the American College of Neuropsychopharmacology 2003; 28(9): 1553-61.

Peng C H, Chiou S H, Chen S J, Chou Y C, Ku H H, Cheng C K, et al. Neuroprotection by Imipramine against lipopolysaccharide-induced apoptosis in hippocampus-derived neural stem cells mediated by activation of BDNF and the MAPK pathway. European neuropsychopharmacology : the journal of the European College of Neuropsychopharmacology 2008; 18(2): 128-40.

Prineas J W, Kwon E E, Cho E S, Sharer L R, Barnett M H, Oleszak E L, et aL Immunopathology of secondary-progressive multiple sclerosis. Ann Neurol 2001; 50(5): 646-57.

Ransohoff RM, Hafler DA, Lucchinetti CF. Multiple sclerosis-a quiet revolution. Nat Rev Neurol 2015; 11(3): 134-42.

Rodriguez de la Torre B, Dreher J, Malevany I, Bagli M, Kolbinger M, Omran H, et al. Serum levels and cardiovascular effects of tricyclic antidepressants and selective serotonin reuptake inhibitors in depressed patients. Therapeutic drug monitoring 2001; 23(4): 435-40.

Romme Christensen J, Bornsen L, Ratzer R, Piehl F, Khademi M, Olsson T, et al. Systemic inflammation in progressive multiple sclerosis involves follicular T-helper, Th17- and activated B-cells and correlates with progression. PloS one 2013; 8(3): e57820.

Samanani S, Mishra M, Silva C, Verhaeghe B, Wang J, Tong J, et al. Screening for inhibitors of microglia to reduce neuroinflammation. CNS & neurological disorders drug targets 2013; 12(6): 741-9.

Schreiber S, Bader M, Rubovitch V, Pick C G. Interaction between methylphenidate, methadone and different antidepressant drugs on antinociception in mice, and possible clinical implications. The world journal of biological psychiatry : the official journal of the World Federation of Societies of Biological Psychiatry 2015: 1-8.

Serafini B, Rosicarelli B, Magliozzi R, Stigliano E, Aloisi F. Detection of ectopic B-cell follicles with germinal centers in the meninges of patients with secondary progressive multiple sclerosis. Brain pathology (Zurich, Switzerland) 2004; 14(2): 164-74.

Stephenson E, Nathoo N, Mahjoub Y, Dunn JF, Yong VW. Iron in multiple sclerosis: roles in neurodegeneration and repair. Nat Rev Neurol 2014; 10(8): 459-68.

Tang T S, Slow E, Lupu V, Stavrovskaya IG, Sugimori M, Llinas R, et al. Disturbed Ca2+ signaling and apoptosis of medium spiny neurons in Huntington's disease. Proceedings of the National Academy of Sciences of the United States of America 2005; 102(7): 2602-7.

Thoren P, Asberg M, Bertilsson L, Mellstrom B, Sjoqvist F, Traskman L. Clomipramine treatment of obsessive-compulsive disorder. II. Biochemical aspects. Archives of general psychiatry 1980; 37(11): 1289-94.

Vecil G G, Larsen P H, Corley S M, Herx L M, Besson A, Goodyer C G, et al. Interleukin-1 is a key regulator of matrix metalloproteinase-9 expression in human neurons in culture and following mouse brain trauma in vivo. Journal of neuroscience research 2000; 61(2): 212-24.

Zhu J, Bengtsson B O, Mix E, Ekerling L, Thorell L H, Olsson T, et al. Clomipramine and imipramine suppress clinical signs and T and B cell response to myelin proteins in experimental autoimmune neuritis in Lewis rats. Journal of autoimmunity 1998; 11(4): 319-27.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A kit for the treatment of progressive multiple sclerosis, comprising:
a) one or more of clomipramine, or functional derivative thereof; or
b) indapamide, or a functional derivative thereof, and one or more of hydroxychloroquine, minocycline, or clomipramine or a functional derivative thereof; and
c) Instructions for the use thereof.

2. The kit of claim 2, further comprising one or more of Laquinimod, Fingolimod, Masitinib, Ocrelizumab, Ibudilast, Anti-LINGO-1, MD1003 (high concentration Biotin), Natalizumab, Siponimod, Tcelna (imilecleucel-T), Simvastatin, Dimethyl fumarate, Autologous haematopoietic stem cell transplantation, Amiloride, Riluzole, Fluoxetine, Glatiramer Acetate, Interferon Beta, or a functional derivative thereof.
